SEQUENCE LISTING (80 sequences)

SEQ ID NO: 1 - protein, 8 aa, synthetic construct
GYTFTSYW

SEQ ID NO: 2 - protein, 8 aa, synthetic construct
IYPGSGST

SEQ ID NO: 3 - protein, 11 aa, synthetic construct
APYDSDYAMDY

SEQ ID NO: 4 - protein, 6 aa, synthetic construct
QDINNY

SEQ ID NO: 5 - DNA, 1359 bp, synthetic construct
  source 1..1359: mol_type = other DNA
gaagttcagctgcagcagtctggggcagaacttgtgaagccaggggcctcagtcaagttgtcctgcacagcttctggcttcaacattgaagactactatatacactgggtgaaggagaggactgaacagggcctggagtggattggaaggattgatcctgaggatggtgaaactacatatgccccgcagttccagggcaaggccactataataccagacacatcctccaacacagcctacatgcagctcagcagcctgacatctgaggacgctgccgtctattactgtgctagatcgatctaccttgatcctatggactactggggtcaaggaacctcagtcaccgtctcctcaaagaccacacctcctagcgtgtaccctctggctcctggctgtggcgatacaacaggcagctctgtgacactgggctgcctggtcaagggctactttcctgagagcgtgacagtgacctggaacagcggcagcctgtctagcagcgtgcacacctttccagctctgctccagagcggcctgtacaccatgtcctctagtgtgaccgtgcctagcagcacctggcctagccagacagtgacatgtagcgtggcccatcctgccagcagcacaaccgtggacaagaagctggaacctagcggccccatcagcaccatcaatccctgtcctccatgcaaagaatgccacaagtgccccgctcctaacctggaaggtggcccaagcgtgttcatcttcccacctaacatcaaggacgtgctgatgatcagcctgacacctaaagtgacctgcgtggtggtggacgtgtccgaggatgatcccgatgtgcagatcagttggttcgtgaacaacgtggaagtgcacacagcccagacacagacccacagagaggactacaatagcaccattcgcgtggtgtccacactgcctatccagcaccaggattggatgagcggcaaagagttcaagtgcaaagtgaacaacaaggacctgccttctccaatcgagcggaccatcagcaagatcaagggactcgtcagagcccctcaggtgtacatcttgcctccaccagccgagcagctgagcagaaaggatgtgtccctgacctgtctggtcgtgggcttcaaccctggcgacatcagcgtggaatggaccagcaatggccacaccgaggaaaactacaaggacacagcccctgtgctggacagcgacggcagctacttcatctacagcaagctgaacatgaagaccagcaagtgggagaaaaccgacagcttctcctgcaacgtgcggcacgagggcctgaagaactactacctgaagaaaaccatctctcggagccccggcaag

SEQ ID NO: 6 - protein, 9 aa, synthetic construct
LQYDEFPLT

SEQ ID NO: 7 - protein, 118 aa, synthetic construct
QVQLQQPGAELAKPGASVKMSCKASGYTFTSYWITWVKQRPGQDLEWIGNIYPGSGSTYYNEKFKSKATLTVDTSSRTAYMQLSSLTSADSAVYYCAPYDSDYAMDYWGQGTSVTVSS

SEQ ID NO: 8 - protein, 107 aa, synthetic construct
DIKMTQSPSSMYASLGERVTITCKASQDINNYLSWFQQKPGKSPKTLIYRANRLVDGVPSRVSGSGSGQDYSLTISSLEYEDVGIYFCLQYDEFPLTFGAGTKLELK

SEQ ID NO: 9 - protein, 329 aa, synthetic construct
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 10 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - protein, 447 aa, synthetic construct
QVQLQQPGAELAKPGASVKMSCKASGYTFTSYWITWVKQRPGQDLEWIGNIYPGSGSTYYNEKFKSKATLTVDTSSRTAYMQLSSLTSADSAVYYCAPYDSDYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 12 - protein, 214 aa, synthetic construct
DIKMTQSPSSMYASLGERVTITCKASQDINNYLSWFQQKPGKSPKTLIYRANRLVDGVPSRVSGSGSGQDYSLTISSLEYEDVGIYFCLQYDEFPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 13 - protein, 8 aa, synthetic construct
GFNIKDYY

SEQ ID NO: 14 - protein, 8 aa, synthetic construct
IDPEDGES

SEQ ID NO: 15 - protein, 13 aa, synthetic construct
TRGDSMMVTYFDY

SEQ ID NO: 16 - protein, 6 aa, synthetic construct
QDVSTA

SEQ ID NO: 17 - protein, 456 aa, synthetic construct
EVQLQQSGAELVKPGASVKLSCTASGFNIKDYYIHWVKERTEQGLEWFGRIDPEDGESEYAPKFQGKATLTADTSSNTAYLQLSSLTSEDTAVYYCTRGDSMMVTYFDYWGQGTTLTVSSEKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 18 - protein, 9 aa, synthetic construct
QQHYRSPWT

SEQ ID NO: 19 - protein, 121 aa, synthetic construct
EVQLQQSGAELVKPGASVKLSCTASGFNIKDYYIHWVKERTEQGLEWFGRIDPEDGESEYAPKFQGKATLTADTSSNTAYLQLSSLTSEDTAVYYCTRGDSMMVTYFDYWGQGTTLTVSSE

SEQ ID NO: 20 - protein, 107 aa, synthetic construct
DIVMTQSHKFMSTSVGDRVSITCKASQDVSTAVAWYQQKPGQSPKLLIYWAFTRHTGVPDRFTSTGSGTDYALTISSVQAEDLALYYCQQHYRSPWTFGGGTKLEIK

SEQ ID NO: 21 - protein, 335 aa, synthetic construct
KTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 22 - protein, 455 aa, synthetic construct
EVQLQQSGAELVKPGASVKLSCTASGFNIKDYYIHWVKERTEQGLEWFGRIDPEDGESEYAPKFQGKATLTADTSSNTAYLQLSSLTSEDTAVYYCTRGDSMMVTYFDYWGQGTTLTVSSKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 23 - protein, 214 aa, synthetic construct
DIVMTQSHKFMSTSVGDRVSITCKASQDVSTAVAWYQQKPGQSPKLLIYWAFTRHTGVPDRFTSTGSGTDYALTISSVQAEDLALYYCQQHYRSPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 24 - protein, 8 aa, synthetic construct
GFNIEDYY

SEQ ID NO: 25 - protein, 8 aa, synthetic construct
IDPEDGET

SEQ ID NO: 26 - protein, 11 aa, synthetic construct
ARSIYLDPMDY

SEQ ID NO: 27 - protein, 6 aa, synthetic construct
QDVTTA

SEQ ID NO: 28 - DNA, 321 bp, synthetic construct
  source 1..321: mol_type = other DNA
gacatcgtgatgacccagagccacaagttcatgagcaccagcgtgggcgacagagtgtccatcacctgtaaagccagccaggacgtgacaacagccgtggcctggtatcagcagaagcctggccagtctcctaagatcctgatctactgggccaccaccagacacaccggcgtgccagatagattcaccggcagcatcagcggcaccacctacatcctgacaatcagctctgtgcaggccgaggatctggccctgtactactgtcagcagcactacagcaccccttacacctttggcggaggcaccaagctggaaatcaag

SEQ ID NO: 29 - protein, 9 aa, synthetic construct
QQHYSTPYT

SEQ ID NO: 30 - protein, 118 aa, synthetic construct
EVQLQQSGAELVKPGASVKLSCTASGFNIEDYYIHWVKERTEQGLEWIGRIDPEDGETTYAPQFQGKATIIPDTSSNTAYMQLSSLTSEDAAVYYCARSIYLDPMDYWGQGTSVTVSS

SEQ ID NO: 31 - protein, 107 aa, synthetic construct
DIVMTQSHKFMSTSVGDRVSITCKASQDVTTAVAWYQQKPGQSPKILIYWATTRHTGVPDRFTGSISGTTYILTISSVQAEDLALYYCQQHYSTPYTFGGGTKLEIK

SEQ ID NO: 32 - DNA, 642 bp, synthetic construct
  source 1..642: mol_type = other DNA
gacattgtgatgacccagtctcacaaattcatgtccacatcagtaggagacagggtcagcatcacctgcaaggccagtcaggatgtgactactgctgtcgcctggtatcaacaaaaaccaggacagtctcctaaaatactgatttactgggcaaccacccggcacactggagtccctgatcgcttcacaggcagtatatctgggacaacttatattctcaccatcagtagtgtgcaggctgaagacctggcactttattactgtcagcaacattatagcactccgtacacgttcggaggggggaccaagctggaaataaaaagagctgacgccgctcctaccgtgtccatctttccacctagcagcgagcagctgacaagcggcggagccagcgtcgtgtgcttcctgaacaacttctaccccaaggacatcaacgtgaagtggaagatcgacggcagcgagagacagaacggcgtgctgaatagctggaccgaccaggacagcaaggactccacctacagcatgtccagcacactgaccctgaccaaggacgagtacgagcggcacaacagctacacatgcgaggccacacacaagaccagcacaagccccatcgtgaagtccttcaaccggaacgagtgc

SEQ ID NO: 33 - protein, 453 aa, synthetic construct
EVQLQQSGAELVKPGASVKLSCTASGFNIEDYYIHWVKERTEQGLEWIGRIDPEDGETTYAPQFQGKATIIPDTSSNTAYMQLSSLTSEDAAVYYCARSIYLDPMDYWGQGTSVTVSSKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 34 - protein, 214 aa, synthetic construct
DIVMTQSHKFMSTSVGDRVSITCKASQDVTTAVAWYQQKPGQSPKILIYWATTRHTGVPDRFTGSISGTTYILTISSVQAEDLALYYCQQHYSTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 35 - protein, 11 aa, synthetic construct
APYDADYAMDY

SEQ ID NO: 36 - protein, 6 aa, synthetic construct
QDISNY

SEQ ID NO: 37 - protein, 118 aa, synthetic construct
EVQLVQSGAEVKKPGASVKVSCKASGYTFTSYWITWVRQAPGQRLEWIGNIYPGSGSTYYNEKFKSKATITRDTSSRTAYMELSSLRSEDTAVYYCAPYDADYAMDYWGQGTLVTVSS

SEQ ID NO: 38 - protein, 107 aa, synthetic construct
DIQMTQSPSSLSASVGDRVTITCKASQDISNYLSWFQQKPGKAPKLLIYRANRLVEGVPSRFSGSGSGTDFTLTISSLQPEDFATYFCLQYDEFPLTFGGGTKVEIK

SEQ ID NO: 39 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 107 aa, synthetic construct [sequence omitted]

SEQ ID NO: 41 - protein, 447 aa, synthetic construct
EVQLVQSGAEVKKPGASVKVSCKASGYTFTSYWITWVRQAPGQRLEWIGNIYPGSGSTYYNEKFKSKATITRDTSSRTAYMELSSLRSEDTAVYYCAPYDADYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 214 aa, synthetic construct
DIQMTQSPSSLSASVGDRVTITCKASQDISNYLSWFQQKPGKAPKLLIYRANRLVEGVPSRFSGSGSGTDFTLTISSLQPEDFATYFCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 8 aa, synthetic construct
IDPEDAES

SEQ ID NO: 44 - protein, 120 aa, synthetic construct
EVQLVQSGAEVKKPGASVKVSCKASGFNIKDYYIHWVRQATGQGLEWMGRIDPEDAESEYAPKFQGRVTITADTSTDTAYMELSSLRSEDTAVYYCTRGDSMMVTYFDYWGQGTLVTVSS

SEQ ID NO: 45 - protein, 107 aa, synthetic construct
DIQMTQSPSSLSASVGDRVTITCKASQDVSTAVAWYQQKPGKAPKLLIYWAFTRHTGVPSRFSGSGSGTDYALTISSLQPEDFATYYCQQHYRSPWTFGGGTKVEIK

SEQ ID NO: 46 - protein, 449 aa, synthetic construct
EVQLVQSGAEVKKPGASVKVSCKASGFNIKDYYIHWVRQATGQGLEWMGRIDPEDAESEYAPKFQGRVTITADTSTDTAYMELSSLRSEDTAVYYCTRGDSMMVTYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 214 aa, synthetic construct
DIQMTQSPSSLSASVGDRVTITCKASQDVSTAVAWYQQKPGKAPKLLIYWAFTRHTGVPSRFSGSGSGTDYALTISSLQPEDFATYYCQQHYRSPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 8 aa, synthetic construct
IDPEDAET

SEQ ID NO: 49 - protein, 118 aa, synthetic construct
QVQLVQSGAEVKKPGASVKVSCKASGFNIEDYYMHWVRQAPGQRLEWMGRIDPEDAETTYSPKFQGRVTIIPDTSANTAYMELSSLRSEDTAVYYCARSIYLDPMDYWGQGTLVTVSS

SEQ ID NO: 50 - protein, 107 aa, synthetic construct
DIQMTQSPKSLSASVGDRVTITCRASQDVTTALAWYQQKPGQSPKLLIYWATTRHSGVPSRFSGSGSGTDFTLTISSLQPEDFATYYCQQHYSTPYTFGQGTKLEIK

SEQ ID NO: 51 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 447 aa, synthetic construct
QVQLVQSGAEVKKPGASVKVSCKASGFNIEDYYMHWVRQAPGQRLEWMGRIDPEDAETTYSPKFQGRVTIIPDTSANTAYMELSSLRSEDTAVYYCARSIYLDPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 214 aa, synthetic construct
DIQMTQSPKSLSASVGDRVTITCRASQDVTTALAWYQQKPGQSPKLLIYWATTRHSGVPSRFSGSGSGTDFTLTISSLQPEDFATYYCQQHYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - DNA, 354 bp, synthetic construct
  source 1..354: mol_type = other DNA
caggtccaactgcagcagcctggggctgagcttgcgaagcctggggcttcagtgaagatgtcctgcaaggcttctggctacaccttcaccagctactggataacctgggtgaagcagaggcctggacaagaccttgagtggattggaaatatttatcctggtagtggtagtacttactacaatgagaagttcaagagcaaggccacactgactgtagacacatcctccagaacagcctacatgcagctcagcagtctgacatctgcggactctgcggtctattactgtgccccctatgattccgactatgctatggactactggggtcaaggaacctcagtcaccgtctcctca

SEQ ID NO: 55 - DNA, 321 bp, synthetic construct
  source 1..321: mol_type = other DNA
gacatcaagatgacccagtctccatcttccatgtatgcatctctaggagagagagtcactatcacttgcaaggcgagtcaggacattaataactatttaagctggttccagcagaaaccagggaaatctcctaagaccctgatctatcgtgcaaacagattggtagatggggtcccatcaagggtcagtggcagtggatctgggcaagattattctctcaccatcagcagcctggagtatgaagatgtgggaatttatttttgtctacagtatgatgagtttcctctcacgttcggtgctgggaccaagctggagctgaaa

SEQ ID NO: 56 - DNA, 1347 bp, synthetic construct
  source 1..1347: mol_type = other DNA
caggtccaactgcagcagcctggggctgagcttgcgaagcctggggcttcagtgaagatgtcctgcaaggcttctggctacaccttcaccagctactggataacctgggtgaagcagaggcctggacaagaccttgagtggattggaaatatttatcctggtagtggtagtacttactacaatgagaagttcaagagcaaggccacactgactgtagacacatcctccagaacagcctacatgcagctcagcagtctgacatctgcggactctgcggtctattactgtgccccctatgattccgactatgctatggactactggggtcaaggaacctcagtcaccgtctcctcagctaaaacaacagccccatcggtctatccactggcccctgtgtgtggagatacaactggctcctcggtgactctaggatgcctggtcaagggttatttccctgagccagtgaccttgacctggaactctggttccctgtccagtggtgtgcacaccttcccagctgtcctgcagtctgacctctacaccctcagctcaagcgtgactgtaaccagctcgacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtagtcgttgatgtgagcgaggatgacccagatgtccagatcagctggtttgtgaacaacgtggaagtgcacactgctcagacacagacgcatagagaggattacaacagtactctccgggttgtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaggagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggagaagaagaactgggtggagagaaatagctactcctgttcagtggtccacgagggtctgcacaatcaccacacgactaagagcttctcccggactccgggttagtaa

SEQ ID NO: 57 - DNA, 642 bp, synthetic construct
  source 1..642: mol_type = other DNA
gacatcaagatgacccagtctccatcttccatgtatgcatctctaggagagagagtcactatcacttgcaaggcgagtcaggacattaataactatttaagctggttccagcagaaaccagggaaatctcctaagaccctgatctatcgtgcaaacagattggtagatggggtcccatcaagggtcagtggcagtggatctgggcaagattattctctcaccatcagcagcctggagtatgaagatgtgggaatttatttttgtctacagtatgatgagtttcctctcacgttcggtgctgggaccaagctggagctgaaaagagctgacgccgctcctaccgtgtccatctttccacctagcagcgagcagctgacaagcggcggagccagcgtcgtgtgcttcctgaacaacttctaccccaaggacatcaacgtgaagtggaagatcgacggcagcgagagacagaacggcgtgctgaatagctggaccgaccaggacagcaaggactccacctacagcatgtccagcacactgaccctgaccaaggacgagtacgagcggcacaacagctacacatgcgaggccacacacaagaccagcacaagccccatcgtgaagtccttcaaccggaacgagtgc

SEQ ID NO: 58 - DNA, 360 bp, synthetic construct
  source 1..360: mol_type = other DNA
gaggttcagctgcagcagtctggggcagaacttgtgaagccaggggcctcagtcaagttgtcctgcacagcctctggcttcaacattaaagactactatatacactgggtgaaagagaggactgaacagggcctggagtggtttggaaggattgatcctgaggatggtgaaagtgaatatgccccgaaattccagggcaaggccactttaacagcagacacatcctccaatacagcctacctgcagctcagcagcctgacatctgaggacactgccgtctattactgtactagaggagactctatgatggttacctactttgactactggggccaaggcaccactctcacggtctcctca

SEQ ID NO: 59 - DNA, 321 bp, synthetic construct
  source 1..321: mol_type = other DNA
gacattgtgatgacccagtctcacaaattcatgtccacatcagtaggagacagggtcagcatcacctgcaaggccagtcaggatgtgagtactgctgtagcctggtatcaacaaaaaccagggcaatctcctaaactactgatttactgggctttcacccgtcacactggagtccctgatcgcttcacaagcactggatctgggacagattatgctctcaccatcagcagtgtgcaggctgaagacctggcactttattactgtcagcaacattatcgcagtccgtggacgttcggtggaggcaccaaactggaaatcaaa

SEQ ID NO: 60 - DNA, 1365 bp, synthetic construct
  source 1..1365: mol_type = other DNA
gaggttcagctgcagcagtctggggcagaacttgtgaagccaggggcctcagtcaagttgtcctgcacagcctctggcttcaacattaaagactactatatacactgggtgaaagagaggactgaacagggcctggagtggtttggaaggattgatcctgaggatggtgaaagtgaatatgccccgaaattccagggcaaggccactttaacagcagacacatcctccaatacagcctacctgcagctcagcagcctgacatctgaggacactgccgtctattactgtactagaggagactctatgatggttacctactttgactactggggccaaggcaccactctcacggtctcctcaaagaccacacctcctagcgtgtaccctctggctcctggctgtggcgatacaacaggcagctctgtgacactgggctgcctggtcaagggctactttcctgagagcgtgacagtgacctggaacagcggcagcctgtctagcagcgtgcacacctttccagctctgctccagagcggcctgtacaccatgtcctctagtgtgaccgtgcctagcagcacctggcctagccagacagtgacatgtagcgtggcccatcctgccagcagcacaaccgtggacaagaagctggaacctagcggccccatcagcaccatcaatccctgtcctccatgcaaagaatgccacaagtgccccgctcctaacctggaaggtggcccaagcgtgttcatcttcccacctaacatcaaggacgtgctgatgatcagcctgacacctaaagtgacctgcgtggtggtggacgtgtccgaggatgatcccgatgtgcagatcagttggttcgtgaacaacgtggaagtgcacacagcccagacacagacccacagagaggactacaatagcaccattcgcgtggtgtccacactgcctatccagcaccaggattggatgagcggcaaagagttcaagtgcaaagtgaacaacaaggacctgccttctccaatcgagcggaccatcagcaagatcaagggactcgtcagagcccctcaggtgtacatcttgcctccaccagccgagcagctgagcagaaaggatgtgtccctgacctgtctggtcgtgggcttcaaccctggcgacatcagcgtggaatggaccagcaatggccacaccgaggaaaactacaaggacacagcccctgtgctggacagcgacggcagctacttcatctacagcaagctgaacatgaagaccagcaagtgggagaaaaccgacagcttctcctgcaacgtgcggcacgagggcctgaagaactactacctgaagaaaaccatctctcggagccccggcaag

SEQ ID NO: 61 - DNA, 642 bp, synthetic construct
  source 1..642: mol_type = other DNA
gacattgtgatgacccagtctcacaaattcatgtccacatcagtaggagacagggtcagcatcacctgcaaggccagtcaggatgtgagtactgctgtagcctggtatcaacaaaaaccagggcaatctcctaaactactgatttactgggctttcacccgtcacactggagtccctgatcgcttcacaagcactggatctgggacagattatgctctcaccatcagcagtgtgcaggctgaagacctggcactttattactgtcagcaacattatcgcagtccgtggacgttcggtggaggcaccaaactggaaatcaaaagagctgacgccgctcctaccgtgtccatctttccacctagcagcgagcagctgacaagcggcggagccagcgtcgtgtgcttcctgaacaacttctaccccaaggacatcaacgtgaagtggaagatcgacggcagcgagagacagaacggcgtgctgaatagctggaccgaccaggacagcaaggactccacctacagcatgtccagcacactgaccctgaccaaggacgagtacgagcggcacaacagctacacatgcgaggccacacacaagaccagcacaagccccatcgtgaagtccttcaaccggaacgagtgc

SEQ ID NO: 62 - DNA, 354 bp, synthetic construct
  source 1..354: mol_type = other DNA
gaagttcagctgcagcagtctggggcagaacttgtgaagccaggggcctcagtcaagttgtcctgcacagcttctggcttcaacattgaagactactatatacactgggtgaaggagaggactgaacagggcctggagtggattggaaggattgatcctgaggatggtgaaactacatatgccccgcagttccagggcaaggccactataataccagacacatcctccaacacagcctacatgcagctcagcagcctgacatctgaggacgctgccgtctattactgtgctagatcgatctaccttgatcctatggactactggggtcaaggaacctcagtcaccgtctcctca

SEQ ID NO: 63 - DNA, 321 bp, synthetic construct
  source 1..321: mol_type = other DNA
gacattgtgatgacccagtctcacaaattcatgtccacatcagtaggagacagggtcagcatcacctgcaaggccagtcaggatgtgactactgctgtcgcctggtatcaacaaaaaccaggacagtctcctaaaatactgatttactgggcaaccacccggcacactggagtccctgatcgcttcacaggcagtatatctgggacaacttatattctcaccatcagtagtgtgcaggctgaagacctggcactttattactgtcagcaacattatagcactccgtacacgttcggaggggggaccaagctggaaataaaa

SEQ ID NO: 64 - DNA, 1359 bp, synthetic construct
  source 1..1359: mol_type = other DNA
gaggttcagctgcagcagtctggcgccgagcttgtgaaacctggcgcctctgtgaagctgagctgtaccgccagcggcttcaacatcgaggactactacatccactgggtcaaagagcggaccgagcagggactcgagtggatcggaagaatcgaccccgaggacggcgagacaacatacgcccctcagtttcagggcaaagccacaatcatccccgacaccagcagcaacaccgcctacatgcaactgagcagcctgacctctgaagatgccgccgtgtactactgcgcccggtccatctatctggaccccatggattattggggccagggcacaagcgtgaccgtgtcctctaagaccacacctcctagcgtgtaccctctggctcctggctgtggcgatacaacaggcagctctgtgacactgggctgcctggtcaagggctactttcctgagagcgtgacagtgacctggaacagcggcagcctgtctagcagcgtgcacacctttccagctctgctccagagcggcctgtacaccatgtcctctagtgtgaccgtgcctagcagcacctggcctagccagacagtgacatgtagcgtggcccatcctgccagcagcacaaccgtggacaagaagctggaacctagcggccccatcagcaccatcaatccctgtcctccatgcaaagaatgccacaagtgccccgctcctaacctggaaggtggcccaagcgtgttcatcttcccacctaacatcaaggacgtgctgatgatcagcctgacacctaaagtgacctgcgtggtggtggacgtgtccgaggatgatcccgatgtgcagatcagttggttcgtgaacaacgtggaagtgcacacagcccagacacagacccacagagaggactacaatagcaccattcgcgtggtgtccacactgcctatccagcaccaggattggatgagcggcaaagagttcaagtgcaaagtgaacaacaaggacctgccttctccaatcgagcggaccatcagcaagatcaagggactcgtcagagcccctcaggtgtacatcttgcctccaccagccgagcagctgagcagaaaggatgtgtccctgacctgtctggtcgtgggcttcaaccctggcgacatcagcgtggaatggaccagcaatggccacaccgaggaaaactacaaggacacagcccctgtgctggacagcgacggcagctacttcatctacagcaagctgaacatgaagaccagcaagtgggagaaaaccgacagcttctcctgcaacgtgcggcacgagggcctgaagaactactacctgaagaaaaccatctctcggagccccggcaag

SEQ ID NO: 65 - DNA, 642 bp, synthetic construct
  source 1..642: mol_type = other DNA
gacatcgtgatgacccagagccacaagttcatgagcaccagcgtgggcgacagagtgtccatcacctgtaaagccagccaggacgtgacaacagccgtggcctggtatcagcagaagcctggccagtctcctaagatcctgatctactgggccaccaccagacacaccggcgtgccagatagattcaccggcagcatcagcggcaccacctacatcctgacaatcagctctgtgcaggccgaggatctggccctgtactactgtcagcagcactacagcaccccttacacctttggcggaggcaccaagctggaaatcaagagagctgacgccgctcctaccgtgtccatctttccacctagcagcgagcagctgacaagcggcggagccagcgtcgtgtgcttcctgaacaacttctaccccaaggacatcaacgtgaagtggaagatcgacggcagcgagagacagaacggcgtgctgaatagctggaccgaccaggacagcaaggactccacctacagcatgtccagcacactgaccctgaccaaggacgagtacgagcggcacaacagctacacatgcgaggccacacacaagaccagcacaagccccatcgtgaagtccttcaaccggaacgagtgc

SEQ ID NO: 66 - DNA, 354 bp, synthetic construct
  source 1..354: mol_type = other DNA
gaagtgcagctggtgcaatctggcgccgaagtgaagaaacctggcgcctctgtgaaggtgtcctgcaaggcttccggctacacctttaccagctactggatcacctgggtccgacaggctcctggccagagactggaatggatcggcaacatctaccctggctccggctccacctactacaacgagaagttcaagtccaaggccacaatcacccgggacacctcttccagaaccgcctacatggaactgtccagcctgagatctgaggacaccgccgtgtactactgcgccccttacgacgccgactacgccatggattattggggccagggcaccctggtcaccgtgtcctct

SEQ ID NO: 67 - DNA, 321 bp, synthetic construct
  source 1..321: mol_type = other DNA
gacatccagatgacccagtctccatcctctctgtccgcctctgtgggcgacagagtgaccatcacatgcaaggccagccaggacatctccaactacctgtcctggttccagcagaagcctggcaaggctcccaagctgctgatctacagagccaacagactggtggaaggcgtgccctccagattctccggatctggctctggcaccgactttaccctgacaatctccagcctgcagcctgaggacttcgctacctacttctgcctgcaatacgacgagttccctctgacctttggcggaggcaccaaggtggaaatcaag

SEQ ID NO: 68 - DNA, 1341 bp, synthetic construct
  source 1..1341: mol_type = other DNA
gaagtgcagctggtgcaatctggcgccgaagtgaagaaacctggcgcctctgtgaaggtgtcctgcaaggcttccggctacacctttaccagctactggatcacctgggtccgacaggctcctggccagagactggaatggatcggcaacatctaccctggctccggctccacctactacaacgagaagttcaagtccaaggccacaatcacccgggacacctcttccagaaccgcctacatggaactgtccagcctgagatctgaggacaccgccgtgtactactgcgccccttacgacgccgactacgccatggattattggggccagggcaccctggtcaccgtgtcctctgcttctaccaagggacccagcgtgttccctctggctccttccagcaagtctacctctggcggaacagctgctctgggctgcctggtcaaggactactttcctgagcctgtgaccgtgtcttggaactctggcgctctgacatccggcgtgcacacatttccagctgtgctgcagtcctccggcctgtactctctgtcctctgtcgtgaccgtgccttcctctagcctgggcacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacgccagcacctacagagtggtgtccgtgctgacagtgctgcaccaggattggctgaacggcaaagagtacaagtgcaaggtgtccaacaaggccctgcctgctcctatcgaaaagaccatcagcaaggccaagggccagcctagagaaccccaggtttacaccttgcctccatctcgggacgagctgaccaagaaccaggtgtccctgacctgtctcgtgaagggcttctacccctccgacatcgccgtggaatgggagtctaatggccagccagagaacaactacaagacaacccctcctgtgctggactccgacggctcattcttcctgtactccaagctgaccgtggacaagtccagatggcagcagggcaacgtgttctcctgctccgtgatgcacgaggccctgcacaatcactacacacagaagtccctgtctctgtcccctggc

SEQ ID NO: 69 - DNA, 642 bp, synthetic construct
  source 1..642: mol_type = other DNA
gacatccagatgacccagtctccatcctctctgtccgcctctgtgggcgacagagtgaccatcacatgcaaggccagccaggacatctccaactacctgtcctggttccagcagaagcctggcaaggctcccaagctgctgatctacagagccaacagactggtggaaggcgtgccctccagattctccggatctggctctggcaccgactttaccctgacaatctccagcctgcagcctgaggacttcgctacctacttctgcctgcaatacgacgagttccctctgacctttggcggaggcaccaaggtggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 70 - DNA, 360 bp, synthetic construct
  source 1..360: mol_type = other DNA
gaagtgcagctggtgcaatctggcgccgaagtgaagaaacctggcgcctctgtgaaggtgtcctgcaaggcctctggcttcaacatcaaggactactacatccactgggtccgacaggctaccggacagggacttgagtggatgggcagaatcgaccctgaggacgccgagtctgagtacgcccctaagtttcagggcagagtgaccatcaccgccgacacctctaccgacaccgcctacatggaactgtccagcctgagatctgaggacaccgccgtgtactactgcaccagaggcgactccatgatggttacctacttcgactactggggccagggcaccctggtcacagtttcttcc

SEQ ID NO: 71 - DNA, 321 bp, synthetic construct
  source 1..321: mol_type = other DNA
gacatccagatgacccagtctccatcctctctgtccgcctctgtgggcgacagagtgaccatcacatgcaaggcctctcaggacgtgtccaccgccgttgcttggtatcagcagaagcctggcaaggcccctaagctgctgatctactgggccttcaccagacacaccggcgtgccctctaggttctccggctctggctctggcaccgattacgctctgacaatctccagcctgcagcctgaggacttcgccacctactactgccagcagcactacagaagcccctggacatttggcggaggcaccaaggtggaaatcaag

SEQ ID NO: 72 - DNA, 1347 bp, synthetic construct
  source 1..1347: mol_type = other DNA
gaagtgcagctggtgcaatctggcgccgaagtgaagaaacctggcgcctctgtgaaggtgtcctgcaaggcctctggcttcaacatcaaggactactacatccactgggtccgacaggctaccggacagggacttgagtggatgggcagaatcgaccctgaggacgccgagtctgagtacgcccctaagtttcagggcagagtgaccatcaccgccgacacctctaccgacaccgcctacatggaactgtccagcctgagatctgaggacaccgccgtgtactactgcaccagaggcgactccatgatggttacctacttcgactactggggccagggcaccctggtcacagtttcttccgcttccaccaagggacccagcgtgttccctctggctccttccagcaagtctacctctggcggaacagctgctctgggctgcctggtcaaggattacttccctgagcctgtgaccgtgtcctggaactctggcgctctgacatccggcgtgcacacctttccagctgtgctgcaatcctccggcctgtactctctgtcctccgtcgtgaccgtgccttctagctctctgggcacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacgcctccacctacagagtggtgtccgtgctgacagtgctgcaccaggattggctgaacggcaaagagtacaagtgcaaggtgtccaacaaggcactgcccgctcctatcgaaaagaccatctccaaggccaagggccagcctagagaaccccaggtttacaccttgcctccatctcgggacgagctgaccaagaaccaggtgtccctgacctgtctcgtgaagggcttctacccctccgacatcgccgtggaatgggagtctaatggccagccagagaacaactacaagacaacccctcctgtgctggactccgacggctcattcttcctgtactccaagctgaccgtggacaagtccagatggcagcagggcaacgtgttctcctgctccgtgatgcacgaggccctgcacaatcactacacacagaagtctctgtccctgtctcctggc

SEQ ID NO: 73 - DNA, 642 bp, synthetic construct
  source 1..642: mol_type = other DNA
gacatccagatgacccagtctccatcctctctgtccgcctctgtgggcgacagagtgaccatcacatgcaaggcctctcaggacgtgtccaccgccgttgcttggtatcagcagaagcctggcaaggcccctaagctgctgatctactgggccttcaccagacacaccggcgtgccctctaggttctccggctctggctctggcaccgattacgctctgacaatctccagcctgcagcctgaggacttcgccacctactactgccagcagcactacagaagcccctggacatttggcggaggcaccaaggtggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 74 - DNA, 354 bp, synthetic construct
  source 1..354: mol_type = other DNA
caggtgcagctggtgcagtctggcgccgaagtgaaaaagcctggcgcctctgtgaaggtgtcctgcaaggcctctggcttcaacatcgaggactactacatgcactgggtccgacaggcccctggccagagattggaatggatgggcagaatcgaccccgaggacgccgagacaacctactctcctaagttccagggccgcgtgacaatcatccctgacacctctgccaacaccgcctacatggaactgtccagcctgagatctgaggacaccgccgtgtactactgcgcccggtctatctacctggaccctatggactattggggccagggcaccctggtcacagtgtcctct

SEQ ID NO: 75 - DNA, 321 bp, synthetic construct
  source 1..321: mol_type = other DNA
gacatccagatgacccagtctccaaagtctctgtccgcctccgtgggcgacagagtgaccatcacctgtagagcctctcaggacgtgaccaccgctctggcttggtatcagcagaagcctggccagtctcctaagctgctgatctactgggccaccaccagacactctggcgtgccctctagattctccggctctggctctggcaccgactttaccctgacaatctccagcctgcagcctgaggacttcgccacctactactgccagcagcactacagcaccccttacacctttggccagggcaccaagctggaaatcaag

SEQ ID NO: 76 - DNA, 1341 bp, synthetic construct
  source 1..1341: mol_type = other DNA
caggtgcagctggtgcagtctggcgccgaagtgaaaaagcctggcgcctctgtgaaggtgtcctgcaaggcctctggcttcaacatcgaggactactacatgcactgggtccgacaggcccctggccagagattggaatggatgggcagaatcgaccccgaggacgccgagacaacctactctcctaagttccagggccgcgtgacaatcatccctgacacctctgccaacaccgcctacatggaactgtccagcctgagatctgaggacaccgccgtgtactactgcgcccggtctatctacctggaccctatggactattggggccagggcaccctggtcacagtgtcctctgcttctaccaagggacccagcgtgttccctctggctccttccagcaagtctacctctggcggaacagctgctctgggctgcctggtcaaggactactttccagagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaagctgctggcggcccttccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggccaagggccagcctagggaaccccaggtttacaccctgcctccaagccgggaagagatgaccaagaaccaggtgtccctgacctgcctcgtgaagggcttctacccttccgacatcgccgtggaatgggagagcaatggccagccagagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggc

SEQ ID NO: 77 - DNA, 642 bp, synthetic construct
  source 1..642: mol_type = other DNA
gacatccagatgacccagtctccaaagtctctgtccgcctccgtgggcgacagagtgaccatcacctgtagagcctctcaggacgtgaccaccgctctggcttggtatcagcagaagcctggccagtctcctaagctgctgatctactgggccaccaccagacactctggcgtgccctctagattctccggctctggctctggcaccgactttaccctgacaatctccagcctgcagcctgaggacttcgccacctactactgccagcagcactacagcaccccttacacctttggccagggcaccaagctggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 78 - protein, 120 aa, synthetic construct
EVQLQQSGAELVKPGASVKLSCTASGFNIKDYYIHWVKERTEQGLEWFGRIDPEDGESEYAPKFQGKATLTADTSSNTAYLQLSSLTSEDTAVYYCTRGDSMMVTYFDYWGQGTTLTVSS

SEQ ID NO: 79 - DNA, 262 bp, synthetic construct
  source 1..262: mol_type = other DNA
ctactacatccactgggtcaaagagcggaccgagcagggactcgagtggatcggaagaatcgaccccgaggacggcgagacaacatacgcccctcagtttcagggcaaagccacaatcatccccgacaccagcagcaacaccgcctacatgcaactgagcagcctgacctctgaagatgccgccgtgtactactgcgcccggtccatctatctggaccccatggattattggggccagggcacaagcgtgaccgtgtcctct

SEQ ID NO: 80 - protein, 5 aa, synthetic construct
RIVGG

(19) United States
(12) Patent Application Publication
(10) Pub. No.: US 2026/0286007 A1
(43) Pub. Date: Sep. 24, 2026

(54) ANTI-TMPRSS6 ANTIBODIES AND USES THEREOF

(71) Applicant: Disc Medicine, Inc., Watertown, MA (US)

(72) Inventors: Will Savage, Newton Highlands, MA (US); Brian MacDonald, Bend, OR (US); Min Wu, Cambridge, MA (US)

(73) Assignee: Disc Medicine, Inc., Watertown, MA (US)

(21) Appl. No.: 19/472,577

(22) PCT Filed: Apr. 3, 2024
(86) PCT No.: PCT/US2024/022827

§ 371 Date: Oct. 3, 2025

Related U.S. Application Data
(60) Provisional application No. 63/457,081, filed on Apr. 4, 2023.

(51) Int. Cl. C07K 16/40; A61P 7/06

(52) U.S. Cl. CPC C07K 16/40; A61P 7/06

(57) ABSTRACT

Aspects of the disclosure provide anti-TMPRSS6 antibodies and methods of using the same for promoting hepcidin expression, and treating iron overload associated conditions, such as hemochromatosis, sickle cell disease, thalassemia, hemolysis, Diamond-Blackfan anemia, myelodysplastic syndrome (MDS), blood transfusion.

### CROSS REFERENCE TO RELATED APPLICATIONS

This application claims priority to U.S. Provisional Application No. 63/457,081, filed Apr. 4, 2023, which is hereby incorporated by reference in its entirety.

### REFERENCE TO AN ELECTRONIC SEQUENCE LISTING

The contents of the electronic sequence listing (D084270011WO00-SEQ-LJG.xml; Size: 95,921 bytes; and Date of Creation: Apr. 1, 2024) is herein incorporated by reference in its entirety.

### BACKGROUND

Transmembrane serine protease 6 (TMPRSS6; also known as Matriptase-2 or MTP-2) is a type II transmembrane serine protease. TMPRSS6 is highly expressed in the liver and plays a role in iron homeostasis by negatively regulating hepcidin expression. TMPRSS6 cleaves BMP coreceptors (e.g., hemojuvelin (HJV)), which results in decreased expression of hepcidin. Decreased hepcidin expression levels lead to iron overload in various conditions. There is a need for improved compositions and methods for the treatment of iron overload disorders.

### SUMMARY

Certain aspects of the disclosure relate to a recognition that iron overload occurs in various conditions (e.g., hemochromatosis, sickle cell disease (SCD), thalassemia, hemolysis, African iron overload, Diamond-Blackfan anemia, myelodysplastic syndrome (MDS), blood transfusion, etc.). In some embodiments, methods and related compositions provided herein are useful for treating various diseases and conditions (e.g., hemochromatosis, sickle cell disease (SCD), thalassemia, hemolysis, African iron overload, Diamond-Blackfan anemia, myelodysplastic syndrome (MDS), etc.). In some embodiments, methods and related compositions provided herein are useful for inhibition of TMPRSS6 for purposes of reducing iron overload in diseases and conditions described herein. In some embodiments, reducing iron overload ameliorates disease severity. Aspects of the disclosure provide anti-TMPRSS6 antibodies that have high binding affinity and specificity to TMPRSS6 and that inhibit TMPRSS6 activity. Accordingly, in some embodiments, the disclosure provides methods and related anti-TMPRSS6 antibody compositions for treating iron overload associated diseases and conditions, such as hemochromatosis, sickle cell disease (SCD), thalassemia, hemolysis, Diamond-Blackfan anemia, African iron overload, myelodysplastic syndrome (MDS), blood transfusion, etc. In some embodiments, the methods provided herein reduce iron overload in a subject. In some embodiments, the methods provided herein reduce systemic iron level. In some embodiments, the methods provided herein reduce mean corpuscular hemoglobin concentration (MCHC) in a subject.

In some embodiments, the disclosure provides methods for treating sickle cell disease (SCD) by administering to a subject an effective amount of an anti-TMPRSS6 antibody. In some aspects, the present disclosure provides methods and compositions for treating iron overload in a subject having SCD by administering the subject an effective amount of an anti-TMPRSS6 antibody. In some embodiments, methods provided herein result in a reduction in the frequency of painful crises (i.e., vasoocclusive crisis, VOC), severity of VOC (e.g., hospitalization and duration), systemic iron level, mean corpuscular hemoglobin concentration (MCHC), hemoglobin S (HbS) polymerization, blood transfusion, and/or hemolysis in subjects having SCD (e.g., hemoglobin SC disease). In some embodiments, the methods provided herein reduce the frequency of painful crises (VOC) for hemoglobin SC Disease.

In some aspects, the present disclosure provides a method for treating sickle cell disease (SCD), the method comprising administering to a subject an effective amount of an anti-Transmembrane serine protease 6 (TMPRSS6) antibody comprising:
(a) a HC CDR1, HC CDR2 and HC CDR3 of a heavy chain variable domain having the amino acid sequence of SEQ ID NO: 49, and a LC CDR1, LC CDR2 and LC CDR3 of a light chain variable domain having the amino acid sequence of SEQ ID NO: 50;
(b) a HC CDR1, HC CDR2 and HC CDR3 of a heavy chain variable domain having the amino acid sequence of SEQ ID NO: 7, and a LC CDR1, LC CDR2 and LC CDR3 of a light chain variable domain having the amino acid sequence of SEQ ID NO: 8;
(c) a HC CDR1, HC CDR2 and HC CDR3 of a heavy chain variable domain having the amino acid sequence of SEQ ID NOs: 19 or 78, and a LC CDR1, LC CDR2 and LC CDR3 of a light chain variable domain having the amino acid sequence of SEQ ID NO: 20;
(d) a HC CDR1, HC CDR2 and HC CDR3 of a heavy chain variable domain having the amino acid sequence of SEQ ID NO: 30, and a LC CDR1, LC CDR2 and LC CDR3 of a light chain variable domain having the amino acid sequence of SEQ ID NO: 31;
(e) a HC CDR1, HC CDR2 and HC CDR3 of a heavy chain variable domain having the amino acid sequence of SEQ ID NO: 37, and a LC CDR1, LC CDR2 and LC CDR3 of a light chain variable domain having the amino acid sequence of SEQ ID NO: 38; or
(f) a HC CDR1, HC CDR2 and HC CDR3 of a heavy chain variable domain having the amino acid sequence of SEQ ID NO: 44, and a LC CDR1, LC CDR2 and LC CDR3 of a light chain variable domain having the amino acid sequence of SEQ ID NO: 45.

In some embodiments, the anti-TMPRSS6 antibody comprises a HC CDR1, HC CDR2 and HC CDR3 of a heavy chain variable domain having the amino acid sequence of SEQ ID NO: 49, and a LC CDR1, LC CDR2 and LC CDR3 of a light chain variable domain having the amino acid sequence of SEQ ID NO: 50.

In some embodiments, the anti-TMPRSS6 antibody comprises:
(a) a HC CDR1 having the amino acid sequence of SEQ ID NO: 24, a HC CDR2 having the amino acid sequence of SEQ ID NO: 48, a HC CDR3 having the amino acid sequence of SEQ ID NO: 26, a LC CDR1 having the amino acid sequence of SEQ ID NO: 27, a LC CDR2 having the amino acid sequence of WAT, and a LC CDR3 having the amino acid sequence of SEQ ID NO: 29;
(b) a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 4, a LC CDR2 having the amino acid sequence of RAN, and a LC CDR3 having the amino acid sequence of SEQ ID NO: 6;
(c) a HC CDR1 having the amino acid sequence of SEQ ID NO: 13, a HC CDR2 having the amino acid sequence of SEQ ID NO: 14, a HC CDR3 having the amino acid sequence of SEQ ID NO: 15, a LC CDR1 having the amino acid sequence of SEQ ID NO: 16, a LC CDR2 having the amino acid sequence of WAF, and a LC CDR3 having the amino acid sequence of SEQ ID NO: 18;
(d) a HC CDR1 having the amino acid sequence of SEQ ID NO: 24, a HC CDR2 having the amino acid sequence of SEQ ID NO: 25, a HC CDR3 having the amino acid sequence of SEQ ID NO: 26, a LC CDR1 having the amino acid sequence of SEQ ID NO: 27, a LC CDR2 having the amino acid sequence of WAT, and a LC CDR3 having the amino acid sequence of SEQ ID NO: 29;
(e) a HC CDR1 having the amino acid sequence of SEQ ID NO: 1, a HC CDR2 having the amino acid sequence of SEQ ID NO: 2, a HC CDR3 having the amino acid sequence of SEQ ID NO: 35, a LC CDR1 having the amino acid sequence of SEQ ID NO: 36, a LC CDR2 having the amino acid sequence of RAN, and a LC CDR3 having the amino acid sequence of SEQ ID NO: 6; or
(f) a HC CDR1 having the amino acid sequence of SEQ ID NO: 13, a HC CDR2 having the amino acid sequence of SEQ ID NO: 43, a HC CDR3 having the amino acid sequence of SEQ ID NO: 15, a LC CDR1 having the amino acid sequence of SEQ ID NO: 16, a LC CDR2 having the amino acid sequence of WAF, and a LC CDR3 having the amino acid sequence of SEQ ID NO: 18.

In some embodiments, the anti-TMPRSS6 antibody comprises a HC CDR1 having the amino acid sequence of SEQ ID NO: 24, a HC CDR2 having the amino acid sequence of SEQ ID NO: 48, a HC CDR3 having the amino acid sequence of SEQ ID NO: 26, a LC CDR1 having the amino acid sequence of SEQ ID NO: 27, a LC CDR2 having the amino acid sequence of WAT, and a LC CDR3 having the amino acid sequence of SEQ ID NO: 29.

In some embodiments, the anti-TMPRSS6 antibody comprises:
(a) a heavy chain variable domain having the amino acid sequence of SEQ ID NO: 49, and a light chain variable domain having the amino acid sequence of SEQ ID NO: 50;
(b) a heavy chain variable domain having the amino acid sequence of SEQ ID NO: 7, and a light chain variable domain having the amino acid sequence of SEQ ID NO: 8;
(c) a heavy chain variable domain having the amino acid sequence of SEQ ID NOs: 19 or 78, and a light chain variable domain having the amino acid sequence of SEQ ID NO: 20;
(d) a heavy chain variable domain having the amino acid sequence of SEQ ID NO: 30, and a light chain variable domain having the amino acid sequence of SEQ ID NO: 31;
(e) a heavy chain variable domain having the amino acid sequence of SEQ ID NO: 37, and a light chain variable domain having the amino acid sequence of SEQ ID NO: 38; or
(f) a heavy chain variable domain having the amino acid sequence of SEQ ID NO: 44, and a light chain variable domain having the amino acid sequence of SEQ ID NO: 45.

In some embodiments, the anti-TMPRSS6 antibody comprises a heavy chain variable domain having the amino acid sequence of SEQ ID NO: 49, and a light chain variable domain having the amino acid sequence of SEQ ID NO: 50.

In some embodiments, the anti-TMPRSS6 antibody comprises:
(a) a heavy chain comprising the amino acid sequence of SEQ ID NO: 52, and a light chain comprising the amino acid sequence of SEQ ID NO: 53;
(b) a heavy chain comprising the amino acid sequence of SEQ ID NO: 11, and a light chain comprising the amino acid sequence of SEQ ID NO: 12;
(c) a heavy chain comprising the amino acid sequence of SEQ ID NOs: 17 or 22, and a light chain comprising the amino acid sequence of SEQ ID NO: 23;
(d) a heavy chain comprising the amino acid sequence of SEQ ID NO: 33, and a light chain comprising the amino acid sequence of SEQ ID NO: 34;
(e) a heavy chain comprising the amino acid sequence of SEQ ID NO: 41, and a light chain comprising the amino acid sequence of SEQ ID NO: 42; or
(f) a heavy chain comprising the amino acid sequence of SEQ ID NO: 46, and a light chain comprising the amino acid sequence of SEQ ID NO: 47.

In some embodiments, the anti-TMPRSS6 antibody comprises a heavy chain comprising the amino acid sequence of SEQ ID NO: 52, and a light chain comprising the amino acid sequence of SEQ ID NO: 53.

In some embodiments, administration of the antibody reduces iron overload in the subject. In some embodiments, the administration of the antibody reduces hemolysis in the subject relative to the subject prior to the administration. In some embodiments, the subject has recurrent moderate to severe vasoocclusive crisis (VOC). In some embodiments, the administration of the antibody reduces frequency of VOC in the subject relative to the subject prior to the administration. In some embodiments, the administration reduces severity of VOC relative to the subject prior to the administration. In some embodiments, the severity of VOC is measured by frequency of hospitalization and/or duration of hospitalization.

In some embodiments, the administration of the antibody reduces systemic iron in the subject prior to the administration. In some embodiments, the administration of the antibody reduces mean corpuscular hemoglobin concentration (MCHC) relative to prior to the administration. In some embodiments, the administration reduces hemoglobin S (HbS) polymerization relative to the subject prior to the administration. In some embodiments, the administration reduces the frequency the subject needs blood transfusion relative to the subject prior to the administration. In some embodiments, the administration reduces inflammation in the subject relative to the subject prior to the administration. In some embodiments, the sickle cell disease is hemoglobin SS disease. In some embodiments, the sickle cell disease is hemoglobin SC disease.

In some embodiments, the anti-TMPRSS6 antibody is administered as a co-therapy in combination hemoglobin S polymerization inhibitor (e.g., voxelotor), therapeutic agents for reducing VOC (e.g., Hydroxyurea, L-glutamine oral powder, Crizanlizumab, selective pyruvate kinase-R (PKR) activator), pain-relieving medications (e.g., narcotics, opioids, gabapentin, cannabis), a blood transfusion, a stem cell transplant, Exagmglogene autotemcel (exa-cel), LentiGlobin, GMI-1070, VIT-2763 (vamifeport), Ticagrelor, Vitamin D, simvastatin, AG-348 (mitapivat sulfate), propranolol, epinephrine, regadenoson, atorvastatin, prasugrel, L-arginine, OTQ923, oxygen therapy, or gene therapy.

In some aspects, the present disclosure provides a method for treating an iron overload associated condition in a subject in need thereof, wherein the subject does not have β-thalassemia intermedia or type I hemochromatosis, the method comprising administering to the subject an effective amount of an anti-Transmembrane serine protease 6 (TMPRSS6) antibody comprising:
(a) a HC CDR1, HC CDR2 and HC CDR3 of a heavy chain variable domain having the amino acid sequence of SEQ ID NO: 49, and a LC CDR1, LC CDR2 and LC CDR3 of a light chain variable domain having the amino acid sequence of SEQ ID NO: 50;
(b) a HC CDR1, HC CDR2 and HC CDR3 of a heavy chain variable domain having the amino acid sequence of SEQ ID NO: 7, and a LC CDR1, LC CDR2 and LC CDR3 of a light chain variable domain having the amino acid sequence of SEQ ID NO: 8;
(c) a HC CDR1, HC CDR2 and HC CDR3 of a heavy chain variable domain having the amino acid sequence of SEQ ID NOs: 19 or 78, and a LC CDR1, LC CDR2 and LC CDR3 of a light chain variable domain having the amino acid sequence of SEQ ID NO: 20;
(d) a HC CDR1, HC CDR2 and HC CDR3 of a heavy chain variable domain having the amino acid sequence of SEQ ID NO: 30, and a LC CDR1, LC CDR2 and LC CDR3 of a light chain variable domain having the amino acid sequence of SEQ ID NO: 31;
(e) a HC CDR1, HC CDR2 and HC CDR3 of a heavy chain variable domain having the amino acid sequence of SEQ ID NO: 37, and a LC CDR1, LC CDR2 and LC CDR3 of a light chain variable domain having the amino acid sequence of SEQ ID NO: 38; or
(f) a HC CDR1, HC CDR2 and HC CDR3 of a heavy chain variable domain having the amino acid sequence of SEQ ID NO: 44, and a LC CDR1, LC CDR2 and LC CDR3 of a light chain variable domain having the amino acid sequence of SEQ ID NO: 45.

In some embodiments, the anti-TMPRSS6 antibody comprises a HC CDR1, HC CDR2 and HC CDR3 of a heavy chain variable domain having the amino acid sequence of SEQ ID NO: 49, and a LC CDR1, LC CDR2 and LC CDR3 of a light chain variable domain having the amino acid sequence of SEQ ID NO: 50

In some embodiments, the anti-TMPRSS6 antibody comprises:
(a) a HC CDR1 having the amino acid sequence of SEQ ID NO: 24, a HC CDR2 having the amino acid sequence of SEQ ID NO: 48, a HC CDR3 having the amino acid sequence of SEQ ID NO: 26, a LC CDR1 having the amino acid sequence of SEQ ID NO: 27, a LC CDR2 having the amino acid sequence of WAT, and a LC CDR3 having the amino acid sequence of SEQ ID NO: 29;
(b) a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 4, a LC CDR2 having the amino acid sequence of RAN, and a LC CDR3 having the amino acid sequence of SEQ ID NO: 6;
(c) a HC CDR1 having the amino acid sequence of SEQ ID NO: 13, a HC CDR2 having the amino acid sequence of SEQ ID NO: 14, a HC CDR3 having the amino acid sequence of SEQ ID NO: 15, a LC CDR1 having the amino acid sequence of SEQ ID NO: 16, a LC CDR2 having the amino acid sequence of WAF, and a LC CDR3 having the amino acid sequence of SEQ ID NO: 18;
(d) a HC CDR1 having the amino acid sequence of SEQ ID NO: 24, a HC CDR2 having the amino acid sequence of SEQ ID NO: 25, a HC CDR3 having the amino acid sequence of SEQ ID NO: 26, a LC CDR1 having the amino acid sequence of SEQ ID NO: 27, a LC CDR2 having the amino acid sequence of WAT, and a LC CDR3 having the amino acid sequence of SEQ ID NO: 29;
(e) a HC CDR1 having the amino acid sequence of SEQ ID NO: 1, a HC CDR2 having the amino acid sequence of SEQ ID NO: 2, a HC CDR3 having the amino acid sequence of SEQ ID NO: 35, a LC CDR1 having the amino acid sequence of SEQ ID NO: 36, a LC CDR2 having the amino acid sequence of RAN, and a LC CDR3 having the amino acid sequence of SEQ ID NO: 6; or
(f) a HC CDR1 having the amino acid sequence of SEQ ID NO: 13, a HC CDR2 having the amino acid sequence of SEQ ID NO: 43, a HC CDR3 having the amino acid sequence of SEQ ID NO: 15, a LC CDR1 having the amino acid sequence of SEQ ID NO: 16, a LC CDR2 having the amino acid sequence of WAF, and a LC CDR3 having the amino acid sequence of SEQ ID NO: 18.

In some embodiments, the anti-TMPRSS6 antibody comprises a HC CDR1 having the amino acid sequence of SEQ ID NO: 24, a HC CDR2 having the amino acid sequence of SEQ ID NO: 48, a HC CDR3 having the amino acid sequence of SEQ ID NO: 26, a LC CDR1 having the amino acid sequence of SEQ ID NO: 27, a LC CDR2 having the amino acid sequence of WAT, and a LC CDR3 having the amino acid sequence of SEQ ID NO: 29. In some embodiments, the anti-TMPRSS6 antibody comprises:
(a) a heavy chain variable domain having the amino acid sequence of SEQ ID NO: 49, and a light chain variable domain having the amino acid sequence of SEQ ID NO: 50;
(b) a heavy chain variable domain having the amino acid sequence of SEQ ID NO: 7, and a light chain variable domain having the amino acid sequence of SEQ ID NO: 8;
(c) a heavy chain variable domain having the amino acid sequence of SEQ ID NOs: 19 or 78, and a light chain variable domain having the amino acid sequence of SEQ ID NO: 20;
(d) a heavy chain variable domain having the amino acid sequence of SEQ ID NO: 30, and a light chain variable domain having the amino acid sequence of SEQ ID NO: 31;
(e) a heavy chain variable domain having the amino acid sequence of SEQ ID NO: 37, and a light chain variable domain having the amino acid sequence of SEQ ID NO: 38; or
(f) a heavy chain variable domain having the amino acid sequence of SEQ ID NO: 44, and a light chain variable domain having the amino acid sequence of SEQ ID NO: 45.

In some embodiments, the anti-TMPRSS6 antibody comprises a heavy chain variable domain having the amino acid sequence of SEQ ID NO: 49, and a light chain variable domain having the amino acid sequence of SEQ ID NO: 50.

In some embodiments, the anti-TMPRSS6 antibody comprises:
(a) a heavy chain comprising the amino acid sequence of SEQ ID NO: 52, and a light chain comprising the amino acid sequence of SEQ ID NO: 53;
(b) a heavy chain comprising the amino acid sequence of SEQ ID NO: 11, and a light chain comprising the amino acid sequence of SEQ ID NO: 12;
(c) a heavy chain comprising the amino acid sequence of SEQ ID NOs: 17 or 22, and a light chain comprising the amino acid sequence of SEQ ID NO: 23;
(d) a heavy chain comprising the amino acid sequence of SEQ ID NO: 33, and a light chain comprising the amino acid sequence of SEQ ID NO: 34;
(e) a heavy chain comprising the amino acid sequence of SEQ ID NO: 41, and a light chain comprising the amino acid sequence of SEQ ID NO: 42; or
(f) a heavy chain comprising the amino acid sequence of SEQ ID NO: 46, and a light chain comprising the amino acid sequence of SEQ ID NO: 47.

In some embodiments, the anti-TMPRSS6 antibody comprises a heavy chain comprising the amino acid sequence of SEQ ID NO: 52, and a light chain comprising the amino acid sequence of SEQ ID NO: 53.

In some embodiments, administration of the antibody reduces systemic iron in the subject relative to prior to the administration. In some embodiments, the administration of the antibody reduces mean corpuscular hemoglobin concentration (MCHC) relative to prior to the administration. In some embodiments, the administration of the antibody reduces iron overload in the subject relative to prior to the administration.

In some embodiments, the subject has hereditary hemochromatosis. In some embodiments, the hereditary hemochromatosis is type 3 hereditary hemochromatosis.

In some embodiments, the subject has or is suspected of having thalassemia. In some embodiments, the thalassemia is α-thalassemia. In some embodiments, the α-thalassemia is α-thalassemia major, α-thalassemia intermedia, or α-thalassemia-minor. In some embodiments, the thalassemia is β-thalassemia. In some embodiments, the β-thalassemia is β-thalassemia major, or β-thalassemia-minor.

In some embodiments, the subject has or is suspected of having transfusion related iron overload. In some embodiments, the subject is receiving blood transfusion due to blood loss. In some embodiments, the subject is receiving repeated blood transfusion due to anemia.

In some embodiments, the subject has hemolytic anemia. In some embodiments, the subject has transfusion dependent hemolytic anemia, pyruvate kinase deficiency hemolytic anemia, thalassemia related hemolytic anemia, or sickle cell disease related hemolytic anemia.

In some embodiments, the subject has African Iron Overload.

In some embodiments, the subject has Diamond-Blackfan anemia.

In some embodiments, the subject has myelodysplastic syndrome (MDS). In some embodiments, the MDS is refractory anemia with ring-sideroblasts (RARS). In some embodiments, the subject has a SF3B1 mutation.

The foregoing and other aspects, implementations, acts, functionalities, features and embodiments of the present teachings can be more fully understood from the following description in conjunction with the accompanying drawings.

### BRIEF DESCRIPTION OF DRAWINGS

FIGS. 1A-1E demonstrate a dose-dependent effect of treating mice with an inhibitory anti-TMPRSS6 antibody. Mice were injected intraperitoneally with 0 (vehicle), 2, 5, or 10 mg/kg anti-TMPRSS6 antibody and monitored over a period of 10 days. Serum antibody (FIG. 1A), iron (FIG. 1B), and hepcidin (FIG. 1C) concentrations were measured over the course of 10 days following injection. FIG. 1D shows the expression of Hamp in the liver and FIG. 1E shows the TSAT % of the mice over the course of the study.

FIGS. 2A-2D show the body, spleen, and liver weights of sickle cell (Townes model) mice administered anti-TMPRSS6 antibody. FIG. 2A shows the progression of total body weight of mice administered 0, 3, or 10 mg/kg anti-TMPRSS6 antibody, stratified into male and female groups (designated M and F, respectively), over the course of 8 weeks. FIG. 2B shows the measurement of spleen weight/body weight of male and female mice 56 days after administration of 0, 3, or 10 mg/kg anti-TMPRSS6 antibody. FIG. 2C shows the measurement of liver weight/body weight of male and female mice 56 days after administration of 0, 3, or 10 mg/kg anti-TMPRSS6 antibody. FIG. 2D shows the liver weight/body weight of all mice (male and female mice combined) 56 days after administration of 0 or 10 mg/kg anti-TMPRSS6 antibody.

FIGS. 3A-3C show the reduction of hemolysis markers in sickle cell mice administered anti-TMPRSS6 antibody. Levels of lactate dehydrogenase (LDH) (FIG. 3A), direct bilirubin (DBili) (FIG. 3B), and total bilirubin (TBili) (FIG. 3C) were measured in sickle cell mice 56 days after administration of 0, 3, or 10 mg/kg anti-TMPRSS6 antibody.

FIGS. 4A-4C show the reduction of systemic inflammation markers in sickle cell mice administered anti-TMPRSS6 antibody. Levels of white blood cells (WBC) as measured by the peroxidase method (FIG. 4A), neutrophils (FIG. 4B), and lymphocytes (FIG. 4C) were measured in the blood of sickle cell mice 56 days after administration of 0, 3, or 10 mg/kg anti-TMPRSS6 antibody.

FIGS. 5A-5C demonstrate reduced HbS polymerization in sickle cell mice treated with anti-TMPRSS6 antibody. Levels of HbS (CHCM) (FIG. 5A), hypochromic red blood cells (FIG. 5B), and microcytic red blood cells (FIG. 5C) were measured in the blood of sickle cell mice 56 days after administration of 0, 3, or 10 mg/kg anti-TMPRSS6 antibody.

### DETAILED DESCRIPTION

The present disclosure, at least in part, relates to a recognition that iron overload occurs in various conditions (e.g., hemochromatosis, sickle cell disease, thalassemia, hemolysis, Diamond-Blackfan anemia, African iron overload, myelodysplastic syndrome (MDS), blood transfusion, etc.). In some embodiments, methods and related compositions provided herein are useful for treating various diseases and conditions (e.g., hemochromatosis, sickle cell disease (SCD), thalassemia, hemolysis, African iron overload, Diamond-Blackfan anemia, myelodysplastic syndrome (MDS), etc.). In some embodiments, methods and related compositions are provided that are useful for inhibition of TMPRSS6 for purposes of reducing iron overload, which ameliorates disease severity. Aspects of the disclosure provide anti-TMPRSS6 antibodies that have high binding affinity and specificity to TMPRSS6 and that inhibit TMPRSS6 activity. Accordingly, in some embodiments, the disclosure provides methods and related anti-TMPRSS6 antibody compositions for treating iron overload associated diseases and conditions, such as hemochromatosis, sickle cell disease (SCD), thalassemia, hemolysis, Diamond-Blackfan anemia, African iron overload, myelodysplastic syndrome (MDS), blood transfusion. In some embodiments, the methods provided herein reduce iron overload.

In some embodiments, the disclosure provides methods for treating sickle cell disease (SCD) by administering to a subject an effective amount of an anti-TMPRSS6 antibody. In some aspects, the present disclosure provides methods and compositions for treating iron overload in a subject having SCD by administering the subject an effective amount of an anti-TMPRSS6 antibody. In some embodiments, methods provided herein result in a reduction in the frequency of painful crises (i.e., vasoocclusive crisis, VOC), severity of VOC (e.g., hospitalization and duration), hemoglobin S (HbS) polymerization, blood transfusion, and/or hemolysis in subjects having SCD (e.g., hemoglobin SC disease). In some embodiments, the methods provided herein reduce the frequency of painful crises (VOC) for hemoglobin Sickle C (SC) Disease.

The foregoing and other aspects, implementations, acts, functionalities, features and embodiments of the present teachings can be more fully understood from the following description in conjunction with the accompanying drawings.

### I. Definitions

And/or: As used herein, the term “and/or” is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Administering: As used herein, the terms, “administer”, “administered”, “administering” or “administration” means to provide an antibody or a composition thereof to a subject in a manner that is physiologically and/or pharmacologically useful (e.g., to treat a condition in the subject).

Affinity Matured Antibody: “Affinity Matured Antibody” is used herein to refer to an antibody with one or more alterations in one or more CDRs, which result in an improvement in the affinity (i.e., KD, kd or ka) of the antibody for a target antigen compared to a parent antibody, which does not possess the alteration(s). Exemplary affinity matured antibodies will have nanomolar or even picomolar affinities for the target antigen. A variety of procedures for producing affinity matured antibodies are known in the art, including the screening of a combinatory antibody library that has been prepared using bio-display. For example, Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154(7): 3310-3319 (1995); and Hawkins et al, J. Mol. Biol., 226:889-896 (1992). Selective mutation at selective mutagenesis positions and at contact or hypermutation positions with an activity-enhancing amino acid residue is described in U.S. Pat. No. 6,914,128 B1.

Antibody: As used herein, the term “antibody” refers to a polypeptide that includes at least one immunoglobulin variable domain or at least one site, e.g., paratope, that specifically binds to an antigen. In some embodiments, an antibody comprises a paratope. In some embodiments, a paratope comprise one or more complementarity determining region (CDRs). In some embodiments, an antibody is a full-length antibody. In some embodiments, an antibody is a chimeric antibody. In some embodiments, an antibody is a humanized antibody. However, in some embodiments, an antibody is a Fab fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or CH3 domain. In some embodiments, the amino acid sequence of the VH domain comprises the amino acid sequence of a human gamma (Y) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information system® http://www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) (Epub), 5:45-60 (2005); Lefranc, M.-P. et al., Nucleic Acids Res., 33: D593-597 (2005); Lefranc, M.-P. et al., Nucleic Acids Res., 37: D1006-1012 (2009); Lefranc, M.-P. et al., Nucleic Acids Res., 43: D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). ee also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

In certain embodiments, there are three CDRs in each of the variable regions of a heavy chain and a light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

In certain embodiments, the CDRs of an antibody may have different amino acid sequences when different definition systems are used (e.g., the IMGT definition, the Kabat definition, or the Chothia definition). A definition system annotates each amino acid in a given antibody sequence (e.g., VH or VL sequence) with a number, and numbers corresponding to the heavy chain and light chain CDRs are provided in Table 2. The CDRs listed in Table 1 are defined in accordance with the Kabat definition. One skilled in the art is able to derive the CDR sequences using the different numbering systems for the anti-TMPRSS6 antibodies provided in Table 1.

TABLE 2
CDR Definitions

| | IMGT¹ | Kabat² | Chothia³ |
|---|---|---|---|
| HC CDR1 | 27-38 | 31-35 | 26-32 |
| HC CDR2 | 56-65 | 50-65 | 53-55 |
| HC CDR3 | 105-116/117 | 95-102 | 96-101 |
| LC CDR1 | 27-38 | 24-34 | 26-32 |
| LC CDR2 | 56-65 | 50-56 | 50-52 |
| LC CDR3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987)

CDR-grafted antibody: The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human TMPRSS6 and non-human primate TMPRSS6) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

Effective Amount: As used herein, “an effective amount” refers to the amount of each active agent (e.g., anti-TMPRSS6 antibody) required to confer therapeutic effect on the subject, either alone or in combination with one or more other active agents. In some embodiments, the therapeutic effect is reduced TMPRSS6 level or activity, increased hepcidin level or activity, and/or alleviated disease conditions (e.g., iron overload in sickle cell disease, thalassemia, hemochromatosis, etc.).

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein. In some embodiments, the sequences of the disclosed framework regions may vary by up to 20% (e.g., the sequences are about 80% (e.g., 80%, 85%, 90%, 91%, 92%, 93%, 94%, 95%, 96%, 97%, 98%, or 99%) identical to the disclosed sequence.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti-TMPRSS6 antibodies and antigen binding portions are provided. Such antibodies may be generated by obtaining murine anti-TMPRSS6 monoclonal antibodies using traditional hybridoma technology followed by humanization using in vitro genetic engineering, such as those disclosed in Kasaian et al PCT publication No. WO 2005/123126 A2.

Humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some embodiments, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs derived from one or more CDRs from the original antibody. Humanized antibodies may also involve affinity maturation.

In some embodiments, humanization is achieved by grafting the CDRs (e.g., as shown in Table 1) into the human variable domains (e.g., IGKV1-NL1*01 and IGHV1-3*01 human variable domain). In some embodiments, the anti-TMPRSS6 antibody of the present disclosure is a humanized variant comprising one or more amino acid substitutions (e.g., in the VH framework region) as compared with any one of the VHs listed in Table 1, and/or one or more amino acid substitutions (e.g., in the VL framework region) as compared with any one of the VLs listed in Table 1.

Isolated antibody: An “isolated antibody”, as used herein, is intended to refer to an antibody that is substantially free of other antibodies having different antigenic specificities (e.g., an isolated antibody that specifically binds TMPRSS6 is substantially free of antibodies that specifically bind antigens other than TMPRSS6). An isolated antibody that specifically binds TMPRSS6 may, however, have cross-reactivity to other antigens. Moreover, an isolated antibody may be substantially free of other cellular material and/or chemicals.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Percent (%) sequence identity: The terms “Percent (%) sequence identity”, “percent (%) identity”, and “percent (%) identity to” with respect to a reference polypeptide (or nucleotide) sequence is defined as the percentage of amino acid residues (or nucleic acids) in a candidate sequence that are identical to the amino acid residues (or nucleic acids) in the reference polypeptide (or nucleotide) sequence, after aligning the sequences and introducing gaps, if necessary, to achieve the maximum percent sequence identity, and not considering any conservative substitutions as part of the sequence identity. Alignment for purposes of determining percent sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (or nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

Recombinant antibody: The term “recombinant antibody”, as used herein, is intended to include all antibodies that are prepared, expressed, created or isolated by recombinant means, such as antibodies expressed using a recombinant expression vector transfected into a host cell (described in more details in this disclosure), including, for example, antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. In some embodiments, recombinant human antibodies are provided herein. In certain embodiments, such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human TMPRSS6 which can be generated using techniques well known in the art, such as, but not limited to, using human Ig phage libraries such as those disclosed in Jermutus et al., PCT publication No. WO 2005/007699 A2.

Selective: As used herein, the term “selective” or “selectively” refers to the ability of a molecule to produce an effect (e.g., inhibit, antagonize, agonize, etc) in relation to its target molecule compared to a reference molecule. For example, a molecule that selectively inhibits its target molecule means that this molecule is capable of inhibiting its target molecule with a degree that is distinguishable from a reference molecule in an inhibition assay or other inhibitory context. For example, with respect to an inhibitor, the term, “selectively inhibits”, refers to the ability of the inhibitor to inhibit its target molecule with a degree that is distinguishable from a reference molecule that is not substantially inhibited in an inhibition assay, e.g., to an extent that permit selective inhibition of the target molecule, as described herein. Once the reaction is terminated, the signal produced by inhibiting the target molecule can be measured. The half maximal inhibitor concentration for the target molecule and the reference molecule can be calculated. In some embodiments, a molecule described herein selectively binds to a target molecule. In some embodiments, a molecule described herein selectively inhibits a target molecule. In some embodiments, a molecule described herein selectively antagonizes a target molecule.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, which enables the antibody to be used to distinguish the specific antigen from others, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds TMPRSS6.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having iron overload and/or one or more conditions arising as a result of iron overload.

Treatment: As used herein, the term “treating”, “treated”, “treat” or “treatment” refers to the application or administration of a composition including one or more active agents (e.g., anti-TMPRSS6 antibodies) to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder. Alleviating a target disease/disorder includes delaying or preventing the onset, development or progression of the disease, or reducing disease severity. In some embodiments, the target disease or disorder of the present disclosure include but are not limited to hemochromatosis, sickle cell disease (SCD), thalassemia, hemolysis, African iron overload, Diamond-Blackfan anemia, myelodysplastic syndrome (MDS), and blood transfusion.

### II. Anti-TMPRSS6 Antibodies

In some embodiments, the anti-TMPRSS6 antibody is an antibody specific for TMPRSS6. Provided herein, in some aspects, are antibodies that bind to human TMPRSS6 with high specificity and affinity. In some embodiments, the anti-TMPRSS6 antibody described herein specifically binds to any extracellular epitope of a TMPRSS6 or an epitope that becomes exposed to an antibody. In some embodiments, anti-TMPRSS6 antibodies provided herein bind specifically to TMPRSS6 from human, non-human primates, mouse, rat, etc. In some embodiments, anti-TMPRSS6 antibodies provided herein bind to human TMPRSS6. In some embodiments, the anti-TMPRSS6 antibody described herein binds to an amino acid segment of a human or non-human primate TMPRSS6. In some embodiments, the anti-TMPRSS6 antibody described herein specifically binds to an epitope on human TMPRSS6.

TMPRSS6, also called Matriptase-2, is a serine protease within the type II transmembrane serine protease (TTSP) family. Enzymes in this family share common structural features, including a serine protease domain, a variable length stem region comprising a mosaic of structural domains, and a short cytoplasmic tail (Hooper et al., Type II transmembrane serine proteases: Insights into an emerging class of cell surface proteolytic enzymes. J Biol Chem. 2001; 276:857-60). The TTSP family of proteases comprises multiple sub-families, including the matriptase sub-family, which comprises TMPRSS 2-5. TMPRSS6, to which the antibodies of some embodiments of the present disclosure specifically bind, is highly conserved across mammalian species. In some embodiments, the antibodies of the present disclosure specifically bind a TMPRSS6 protein encoded by a human TMPRSS6 gene (e.g., GenBank: AAH39082.1, e.g., NCBI Gene ID: 164656). In some embodiments, the antibodies of the present disclosure specifically bind a TMPRSS6 protein encoded by a mouse TMPRSS6 gene (e.g., GenBank: AAH57674.1, e.g., NCBI Gene ID: 71753). In other embodiments, the antibodies of the present disclosure specifically bind a TMPRSS6 protein encoded by a non-human primate TMPRSS6 gene (e.g., cynomolgus monkey (i.e., Macaca fascicularis), e.g., NCBI Gene ID: 102139590). The TMPRSS6 gene spans 18 exons with 17 intervening introns, with the matriptase-2 protein domain boundaries corresponding with intron/exon junctions of the encoding gene across all species. Structurally, matriptase-2 comprises canonical TTSP features, such as a short cytoplasmic amino terminal tail, a transmembrane region, a stem region containing two complement protein subcomponents (C1r/C1s, urchin embryonic growth factor and bone morphogenic protein 1 (CUB) domains), and three low-density lipoprotein receptor class A (LDLR) domains, as well as a trypsin-like serine protease domain at the carboxy terminus. All of these structural features are tightly conserved across human, macaque, dog, cow, mouse and rat, with the human protein sharing 95.6%, 91.1%, 85.6%, 80.1% and 80.4% identity, respectively, to matriptase-2 from these species (Ramsay et al., The type II transmembrane serine protease matriptase-2—identification, structural features, enzymology, expression pattern and potential roles. Front Biosci. 2008; 13:259-79).

The matriptase-2 proteolytic domain comprises a serine protease triad of H, D and S residues required for catalytic activity, and an SWG motif predicted to be located at the top of the substrate S1 binding pocket positioning the scissile bond of the substrate in the correct orientation. Proteolytic activation of matriptase-2 is predicted to occur within a motif (RIVGG (SEQ ID NO: 80)) at the junction of the pro- and catalytic domains, which is characteristic of serine proteases and conserved across species (Velasco et al., Matriptase-2, a membrane-bound mosaic serine proteinase predominantly expressed in human liver and showing degrading activity against extracellular matrix proteins. J Biol Chem. 2002; 277:37637-46). In adult human and mouse tissues, the primary site of matriptase-2 mRNA expression is the liver. Specifically, matriptase-2 mRNA expression has been demonstrated to be restricted to liver hepatocytes.

Functionally, matriptase-2 acts to cleave hemojuvelin (HJV), a protein essential in the process of iron regulation through its positive regulation of hepcidin expression. Hepcidin is a known negative regulator of iron absorption, release, and recycling. In the absence of matriptase-2, HJV levels are increased, leading to increased levels of hepcidin and consequently inhibition of iron transport. The establishment of matriptase-2 as an essential component of iron homeostasis was based on phenotypes of iron-refractory iron deficiency anemia in human patients with loss-of-function TMPRSS6 mutations (Cui et al., Iron-refractory iron deficiency anemia: new molecular mechanisms. Kidney Int. 2009; 76(11):1137-1141). Thus, in some embodiments, the antibodies of the present disclosure inhibit matriptase-2 activity in order to regulate iron homeostasis. In some embodiments, the antibodies of the present disclosure inhibit matriptase-2 activity to decrease iron transport through increased hepcidin activity.

In some embodiments, the anti-TMPRSS6 antibody described herein may bind to a fragment of a human TMPRSS6. The fragment of TMPRSS6 may be between about 5 and about 425 amino acids, between about 10 and about 400 amino acids, between about 50 and about 350 amino acids, between about 100 and about 300 amino acids, between about 150 and about 250 amino acids, between about 200 and about 300 amino acids, or between about 75 and about 150 amino acids in length. In some embodiments, the fragment may comprise a contiguous number of amino acids from TMPRSS6. In other embodiments, the fragment may comprise a non-contiguous number of amino acids from TMPRSS6.

In some embodiments, the anti-TMPRSS6 antibodies disclosed herein specifically bind to mouse TMPRSS6. In some embodiments, the anti-TMPRSS6 antibodies disclosed herein specifically bind rat TMPRSS6. In some embodiments, the anti-TMPRSS6 antibodies disclosed herein specifically bind non-human primate TMPRSS6 (e.g., TMPRSS6 from cynomolgus monkey, rhesus monkey, African green monkey, vervet monkey, squirrel monkey, owl monkey, pig-tailed monkey, or baboon). In some embodiments, the anti-TMPRSS6 antibodies disclosed herein specifically bind to human TMPRSS6.

In some embodiments, the anti-TMPRSS6 antibodies described herein are affinity matured clones. In some embodiments, an anti-TMPRSS6 antibody specifically binds a TMPRSS6 (e.g., a human or non-human primate TMPRSS6) with binding affinity (e.g., as indicated by K_{D}) of at least about 10⁻⁴ M, 10⁻⁵ M, 10⁻⁶ M, 10⁻⁷ M, 8⁻⁷ M, 6⁻⁷ M, 4⁻⁷ M, 2⁻⁷ M, 10⁻⁸ M, 8⁻⁸ M, 6⁻⁸ M, 4⁻⁸ M, 2⁻⁸ M, 10⁻⁹ M, 10⁻¹⁰ M, 10⁻¹¹ M, 10⁻¹² M, 10⁻¹³ M, or less. For example, the anti-TMPRSS6 antibodies of the present disclosure can bind to a TMPRSS6 protein (e.g., human TMPRSS6) with an affinity between 5 μM and 750 nM, e.g., between 1 nM and 500 nM, e.g., between 10 nM and 450 nM, e.g., between 50 μM and 100 nM, e.g., between 500 μM and 50 nM. The disclosure also includes antibodies that compete with any of the antibodies described herein for binding to a TMPRSS6 protein (e.g., human TMPRSS6) and that have an affinity of 500 nM or lower (e.g., 400 nM or lower, 100 nM or lower, 80 nM or lower, 50 nM or lower, 20 nM or lower, 10 nM or lower, 500 μM or lower, 50 μM or lower, or 5 μM or lower). In some embodiments, the anti-TMPRSS6 antibodies described herein binds to TMPRSS6 with a K_{D} of sub-nanomolar range.

The affinity and binding kinetics of the anti-TMPRSS6 antibody can be tested using any suitable method, for example, binding affinity (or binding specificity) can be determined by a variety of methods including, but not limited to, biosensor technology (e.g., OCTET or BIACORE), equilibrium dialysis, equilibrium binding, gel filtration, ELISA, surface plasmon resonance (SPR), florescent activated cell sorting (FACS) or spectroscopy (e.g., using a fluorescence assay). Exemplary conditions for evaluating binding affinity are in HbS-P buffer (10 mM HEPES pH7.4, 150 mM NaCl, 0.005% (v/v) surfactant P20) and PBS buffer (10 mM PO4-3, 137 mM NaCl, and 2.7 mM KCl). These techniques can be used to measure the concentration of bound proteins as a function of target protein concentration. The concentration of bound protein ([[Bound]]) is generally related to the concentration of free target protein ([[Free]]) by the following equation:

[[Bound]]=[[Free]]/(Kd+[[Free]])

It is not always necessary to make an exact determination of KA, though, since sometimes it is sufficient to obtain a quantitative measurement of affinity, e.g., determined using a method such as ELISA or FACS analysis, is proportional to KA, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

The heavy chain (HC) and light chain (LC) sequences, heavy chain variable domain (VH) and light chain variable domain (VL), CDR sequences, and heavy chain and light chain constant region sequences of non-limiting examples of anti-TMPRSS6 antibodies are provided in Table 1.

TABLE 1
Examples of anti-TMPRSS6 antibodies (CDRs according to the Kabat definition)

| TMPRSS6 Antibody | | Sequence | SEQ ID NO |
|---|---|---|---|
| A1 | HC CDR1 | GYTFTSYW | 1 |
| | HC CDR2 | IYPGSGST | 2 |
| | HC CDR3 | APYDSDYAMDY | 3 |
| | LC CDR1 | QDINNY | 4 |
| | LC CDR2 | RAN | N/A |
| | LC CDR3 | LQYDEFPLT | 6 |
| | VH | QVQLQQPGAELAKPGASVKMSCK | 7 |
| | | ASGYTFTSYWITWVKQRPGQDLE | |
| | | WIGNIYPGSGSTYYNEKFKSKAT | |
| | | LTVDTSSRTAYMQLSSLTSADSA | |
| | | VYYCAPYDSDYAMDYWGQGTSVT | |
| | | VSS | |
| | VL | DIKMTQSPSSMYASLGERVTITC | 8 |
| | | KASQDINNYLSWFQQKPGKSPKT | |
| | | LIYRANRLVDGVPSRVSGSGSGQ | |
| | | DYSLTISSLEYEDVGIYFCLQYD | |
| | | EFPLTFGAGTKLELK | |
| | HC Constant | AKTTAPSVYPLAPVCGDTTGSSV | 9 |
| | Region | TLGCLVKGYFPEPVTLTWNSGSL | |
| | | SSGVHTFPAVLQSDLYTLSSSVT | |
| | | VTSSTWPSQSITCNVAHPASSTK | |
| | | VDKKIEPRGPTIKPCPPCKCPAP | |
| | | NLLGGPSVFIFPPKIKDVLMISL | |
| | | SPIVTCVVVDVSEDDPDVQISWF | |
| | | VNNVEVHTAQTQTHREDYNSTLR | |
| | | VVSALPIQHQDWMSGKEFKCKVN | |
| | | NKDLPAPIERTISKPKGSVRAPQ | |
| | | VYVLPPPEEEMTKKQVTLTCMVT | |
| | | DFMPEDIYVEWTNNGKTELNYKN | |
| | | TEPVLDSDGSYFMYSKLRVEKKN | |
| | | WVERNSYSCSVVHEGLHNHHTTK | |
| | | SFSRTPG | |
| | LC Constant | RADAAPTVSIFPPSSEQLTSGGA | 10 |
| | Region | SVVCFLNNFYPKDINVKWKIDGS | |
| | | ERQNGVLNSWTDQDSKDSTYSMS | |
| | | STLTLTKDEYERHNSYTCEATHK | |
| | | TSTSPIVKSFNRNEC | |
| | Heavy Chain | QVQLQQPGAELAKPGASVKMSCK | 11 |
| | | ASGYTFTSYWITWVKQRPGQDLE | |
| | | WIGNIYPGSGSTYYNEKFKSKAT | |
| | | LTVDTSSRTAYMQLSSLTSADSA | |
| | | VYYCAPYDSDYAMDYWGQGTSVT | |
| | | VSSAKTTAPSVYPLAPVCGDTTG | |
| | | SSVTLGCLVKGYFPEPVTLTWNS | |
| | | GSLSSGVHTFPAVLQSDLYTLSS | |
| | | SVTVTSSTWPSQSITCNVAHPAS | |
| | | STKVDKKIEPRGPTIKPCPPCKC | |
| | | PAPNLLGGPSVFIFPPKIKDVLM | |
| | | ISLSPIVTCVVVDVSEDDPDVQI | |
| | | SWFVNNVEVHTAQTQTHREDYNS | |
| | | TLRVVSALPIQHQDWMSGKEFKC | |
| | | KVNNKDLPAPIERTISKPKGSVR | |
| | | APQVYVLPPPEEEMTKKQVTLTC | |
| | | MVTDFMPEDIYVEWTNNGKTELN | |
| | | YKNTEPVLDSDGSYFMYSKLRVE | |
| | | KKNWVERNSYSCSVVHEGLHNHH | |
| | | TTKSFSRTPG | |
| | Light Chain | DIKMTQSPSSMYASLGERVTITC | |
| | | KASQDINNYLSWFQQKPGKSPKT | |
| | | LIYRANRLVDGVPSRVSGSGSGQ | |
| | | DYSLTISSLEYEDVGIYFCLQYD | 12 |
| | | EFPLTFGAGTKLELKRADAAPTV | |
| | | SIFPPSSEQLTSGGASVVCFLNN | |
| | | FYPKDINVKWKIDGSERQNGVLN | |
| | | SWTDQDSKDSTYSMSSTLTLTKD | |
| | | EYERHNSYTCEATHKTSTSPIVK | |
| | | SFNRNEC | |
| B1 | HC CDR1 | GFNIKDYY | 13 |
| | HC CDR2 | IDPEDGES | 14 |
| | HC CDR3 | TRGDSMMVTYFDY | 15 |
| | LC CDR1 | QDVSTA | 16 |
| | LC CDR2 | WAF | N/A |
| | LC CDR3 | QQHYRSPWT | 18 |
| | VH | EVQLQQSGAELVKPGASVKLSCT | 19 |
| | | ASGFNIKDYYIHWVKERTEQGLE | |
| | | WFGRIDPEDGESEYAPKFQGKAT | |
| | | LTADTSSNTAYLQLSSLTSEDTA | |
| | | VYYCTRGDSMMVTYFDYWGQGTT | |
| | | LTVSSE | |
| | VH | EVQLQQSGAELVKPGASVKLSCT | 78 |
| | | ASGFNIKDYYIHWVKERTEQGLE | |
| | | WFGRIDPEDGESEYAPKFQGKAT | |
| | | LTADTSSNTAYLQLSSLTSEDTA | |
| | | VYYCTRGDSMMVTYFDYWGQGTT | |
| | | LTVSS | |
| | VL | DIVMTQSHKFMSTSVGDRVSITC | 20 |
| | | KASQDVSTAVAWYQQKPGQSPKL | |
| | | LIYWAFTRHTGVPDRFTSTGSGT | |
| | | DYALTISSVQAEDLALYYCQQHY | |
| | | RSPWTFGGGTKLEIK | |
| | HC Constant | KTTPPSVYPLAPGCGDTTGSSVT | 21 |
| | Region | LGCLVKGYFPESVTVTWNSGSLS | |
| | | SSVHTFPALLQSGLYTMSSSVTV | |
| | | PSSTWPSQTVTCSVAHPASSTTV | |
| | | DKKLEPSGPISTINPCPPCKECH | |
| | | KCPAPNLEGGPSVFIFPPNIKDV | |
| | | LMISLTPKVTCVVVDVSEDDPDV | |
| | | QISWFVNNVEVHTAQTQTHREDY | |
| | | NSTIRVVSTLPIQHQDWMSGKEF | |
| | | KCKVNNKDLPSPIERTISKIKGL | |
| | | VRAPQVYILPPPAEQLSRKDVSL | |
| | | TCLVVGFNPGDISVEWTSNGHTE | |
| | | ENYKDTAPVLDSDGSYFIYSKLN | |
| | | MKTSKWEKTDSFSCNVRHEGLKN | |
| | | YYLKKTISRSPGK | |
| | LC Constant | RADAAPTVSIFPPSSEQLTSGGA | 10 |
| | Region | SVVCFLNNFYPKDINVKWKIDGS | |
| | | ERQNGVLNSWTDQDSKDSTYSMS | |
| | | STLTLTKDEYERHNSYTCEATHK | |
| | | TSTSPIVKSFNRNEC | |
| | Heavy Chain | EVQLQQSGAELVKPGASVKLSCT | 17 |
| | | ASGFNIKDYYIHWVKERTEQGLE | |
| | | WFGRIDPEDGESEYAPKFQGKAT | |
| | | LTADTSSNTAYLQLSSLTSEDTA | |
| | | VYYCTRGDSMMVTYFDYWGQGTT | |
| | | LTVSSEKTTPPSVYPLAPGCGDT | |
| | | TGSSVTLGCLVKGYFPESVTVTW | |
| | | NSGSLSSSVHTFPALLQSGLYTM | |
| | | SSSVTVPSSTWPSQTVTCSVAHP | |
| | | ASSTTVDKKLEPSGPISTINPCP | |
| | | PCKECHKCPAPNLEGGPSVFIFP | |
| | | PNIKDVLMISLTPKVTCVVVDVS | |
| | | EDDPDVQISWFVNNVEVHTAQTQ | |
| | | THREDYNSTIRVVSTLPIQHQDW | |
| | | MSGKEFKCKVNNKDLPSPIERTI | |
| | | SKIKGLVRAPQVYILPPPAEQLS | |
| | | RKDVSLTCLVVGENPGDISVEWT | |
| | | SNGHTEENYKDTAPVLDSDGSYF | |
| | | IYSKLNMKTSKWEKTDSFSCNVR | |
| | | HEGLKNYYLKKTISRSPGK | |
| | Heavy Chain | EVQLQQSGAELVKPGASVKLSCT | 22 |
| | | ASGFNIKDYYIHWVKERTEQGLE | |
| | | WFGRIDPEDGESEYAPKFQG | |
| | | KATLTADTSSNTAYLQLSSLTSE | |
| | | DTAVYYCTRGDSMMVTYFDYWGQ | |
| | | GTTLTVSSKTTPPSVYPLAPGCG | |
| | | DTTGSSVTLGCLVKGYFPESVTV | |
| | | TWNSGSLSSSVHTFPALLQSGLY | |
| | | TMSSSVTVPSSTWPSQTVTCSVA | |
| | | HPASSTTVDKKLEPSGPISTINP | |
| | | CPPCKECHKCPAPNLEGGPSVFI | |
| | | FPPNIKDVLMISLTPKVTCVVVD | |
| | | VSEDDPDVQISWFVNNVEVHTAQ | |
| | | TQTHREDYNSTIRVVSTLPIQHQ | |
| | | DWMSGKEFKCKVNNKDLPSPIER | |
| | | TISKIKGLVRAPQVYILPPPAEQ | |
| | | LSRKDVSLTCLVVGFNPGDISVE | |
| | | WTSNGHTEENYKDTAPVLDSDGS | |
| | | YFIYSKLNMKTSKWEKTDSFSCN | |
| | | VRHEGLKNYYLKKTISRSPGK | |
| | Light Chain | DIVMTQSHKFMSTSVGDRVSITC | 23 |
| | | KASQDVSTAVAWYQQKPGQSPKL | |
| | | LIYWAFTRHTGVPDRFTSTGSGT | |
| | | DYALTISSVQAEDLALYYCQQHY | |
| | | RSPWTFGGGTKLEIKRADAAPTV | |
| | | SIFPPSSEQLTSGGASVVCFLNN | |
| | | FYPKDINVKWKIDGSERQNGVLN | |
| | | SWTDQDSKDSTYSMSSTLTLTKD | |
| | | EYERHNSYTCEATHKTSTSPIVK | |
| | | SFNRNEC | |
| C1 | HC CDR1 | GFNIEDYY | 24 |
| | HC CDR2 | IDPEDGET | 25 |
| | HC CDR3 | ARSIYLDPMDY | 26 |
| | LC CDR1 | QDVTTA | 27 |
| | LC CDR2 | WAT | N/A |
| | LC CDR3 | QQHYSTPYT | 29 |
| | VH | EVQLQQSGAELVKPGASVKLSCT | 30 |
| | | ASGFNIEDYYIHWVKERTEQGLE | |
| | | WIGRIDPEDGETTYAPQFQGKAT | |
| | | IIPDTSSNTAYMQLSSLTSEDAA | |
| | | VYYCARSIYLDPMDYWGQGTSVT | |
| | | VSS | |
| | VL | DIVMTQSHKFMSTSVGDRVSITC | 31 |
| | | KASQDVTTAVAWYQQKPGQSPKI | |
| | | LIYWATTRHTGVPDRFTGSISGT | |
| | | TYILTISSVQAEDLALYYCQQHY | |
| | | STPYTFGGGTKLEIK | |
| | HC Constant | KTTPPSVYPLAPGCGDTTGSSVT | 21 |
| | Region | LGCLVKGYFPESVTVTWNSGSLS | |
| | | SSVHTFPALLQSGLYTMSSSVTV | |
| | | PSSTWPSQTVTCSVAHPASSTTV | |
| | | DKKLEPSGPISTINPCPPCKECH | |
| | | KCPAPNLEGGPSVFIFPPNIKDV | |
| | | LMISLTPKVTCVVVDVSEDDPDV | |
| | | QISWFVNNVEVHTAQTQTHREDY | |
| | | NSTIRVVSTLPIQHQDWMSGKEF | |
| | | KCKVNNKDLPSPIERTISKIKGL | |
| | | VRAPQVYILPPPAEQLSRKDVSL | |
| | | TCLVVGFNPGDISVEWTSNGHTE | |
| | | ENYKDTAPVLDSDGSYFIYSKLN | |
| | | MKTSKWEKTDSFSCNVRHEGLKN | |
| | | YYLKKTISRSPGK | |
| | LC Constant | RADAAPTVSIFPPSSEQLTSGGA | 10 |
| | Region | SVVCFLNNFYPKDINVKWKIDGS | |
| | | ERQNGVLNSWTDQDSKDSTYSMS | |
| | | STLTLTKDEYERHNSYTCEATHK | |
| | | TSTSPIVKSFNRNEC | |
| | Heavy Chain | EVQLQQSGAELVKPGASVKLSCT | 33 |
| | | ASGFNIEDYYIHWVKERTEQGLE | |
| | | WIGRIDPEDGETTYAPQFQGKAT | |
| | | IIPDTSSNTAYMQLSSLTSEDAA | |
| | | VYYCARSIYLDPMDYWGQGTSVT | |
| | | VSSKTTPPSVYPLAPGCGDTTGS | |
| | | SVTLGCLVKGYFPESVTVTWNSG | |
| | | SLSSSVHTFPALLQSGLYTMSSS | |
| | | VTVPSSTWPSQTVTCSVAHPASS | |
| | | TTVDKKLEPSGPISTINPCPPCK | |
| | | ECHKCPAPNLEGGPSVFIFPPNI | |
| | | KDVLMISLTPKVTCVVVDVSEDD | |
| | | PDVQISWFVNNVEVHTAQTQT | |
| | | HREDYNSTIRVVSTLPIQHQDWM | |
| | | SGKEFKCKVNNKDLPSPIERTIS | |
| | | KIKGLVRAPQVYILPPPAEQLSR | |
| | | KDVSLTCLVVGENPGDISVEWTS | |
| | | NGHTEENYKDTAPVLDSDGSYFI | |
| | | YSKLNMKTSKWEKTDSFSCNVRH | |
| | | EGLKNYYLKKTISRSPGK | |
| | Light Chain | DIVMTQSHKFMSTSVGDRVSITC | 34 |
| | | KASQDVTTAVAWYQQKPGQSPKI | |
| | | LIYWATTRHTGVPDRFTGSISGT | |
| | | TYILTISSVQAEDLALYYCQQHY | |
| | | STPYTFGGGTKLEIKRADAAPTV | |
| | | SIFPPSSEQLTSGGASVVCFLNN | |
| | | FYPKDINVKWKIDGSERQNGVLN | |
| | | SWTDQDSKDSTYSMSSTLTLTKD | |
| | | EYERHNSYTCEATHKTSTSPIVK | |
| | | SFNRNEC | |
| A2 | HC CDR1 | GYTFTSYW | 1 |
| | HC CDR2 | IYPGSGST | 2 |
| | HC CDR3 | APYDADYAMDY | 35 |
| | LC CDR1 | QDISNY | 36 |
| | LC CDR2 | RAN | N/A |
| | LC CDR3 | LQYDEFPLT | 6 |
| | VH | EVQLVQSGAEVKKPGASVKVSCK | 37 |
| | | ASGYTFTSYWITWVRQAPGQRLE | |
| | | WIGNIYPGSGSTYYNEKFKSKAT | |
| | | ITRDTSSRTAYMELSSLRSEDTA | |
| | | VYYCAPYDADYAMDYWGQGTLVT | |
| | | VSS | |
| | VL | DIQMTQSPSSLSASVGDRVTITC | 38 |
| | | KASQDISNYLSWFQQKPGKAPKL | |
| | | LIYRANRLVEGVPSRFSGSGSGT | |
| | | DFTLTISSLQPEDFATYFCLQYD | |
| | | EFPLTFGGGTKVEIK | |
| | HC Constant | ASTKGPSVFPLAPSSKSTSGGTA | 39 |
| | Region | ALGCLVKDYFPEPVTVSWNSGAL | |
| | | TSGVHTFPAVLQSSGLYSLSSVV | |
| | | TVPSSSLGTQTYICNVNHKPSNT | |
| | | KVDKKVEPKSCDKTHTCPPCPAP | |
| | | ELLGGPSVELFPPKPKDTLMISR | |
| | | TPEVTCVVVDVSHEDPEVKFNWY | |
| | | VDGVEVHNAKTKPREEQYASTYR | |
| | | VVSVLTVLHQDWLNGKEYKCKVS | |
| | | NKALPAPIEKTISKAKGQPREPQ | |
| | | VYTLPPSRDELTKNQVSLTCLVK | |
| | | GFYPSDIAVEWESNGQPENNYKT | |
| | | TPPVLDSDGSFFLYSKLTVDKSR | |
| | | WQQGNVFSCSVMHEALHNHYTQK | |
| | | SLSLSPG | |
| | LC Constant | RTVAAPSVFIFPPSDEQLKSGTA | 40 |
| | Region | SVVCLLNNFYPREAKVQWKVDNA | |
| | | LQSGNSQESVTEQDSKDSTYSLS | |
| | | STLTLSKADYEKHKVYACEVTHQ | |
| | | GLSSPVTKSFNRGEC | |
| | Heavy Chain | EVQLVQSGAEVKKPGASVKVSCK | 41 |
| | | ASGYTFTSYWITWVRQAPGQRLE | |
| | | WIGNIYPGSGSTYYNEKFKSKAT | |
| | | ITRDTSSRTAYMELSSLRSEDTA | |
| | | VYYCAPYDADYAMDYWGQGTLVT | |
| | | VSSASTKGPSVFPLAPSSKSTSG | |
| | | GTAALGCLVKDYFPEPVTVSWNS | |
| | | GALTSGVHTFPAVLQSSGLYSLS | |
| | | SVVTVPSSSLGTQTYICNVNHKP | |
| | | SNTKVDKKVEPKSCDKTHTCPPC | |
| | | PAPELLGGPSVFLFPPKPKDTLM | |
| | | ISRTPEVTCVVVDVSHEDPEVKF | |
| | | NWYVDGVEVHNAKTKPREEQYAS | |
| | | TYRVVSVLTVLHQDWLNGKEYKC | |
| | | KVSNKALPAPIEKTISKAKGQPR | |
| | | EPQVYTLPPSRDELTKNQVSLTC | |
| | | LVKGFYPSDIAVEWESNGQPENN | |
| | | YKTTPPVLDSDGSFFLYSKLTVD | |
| | | KSRWQQGNVFSCSVMHEALHNHY | |
| | | TQKSLSLSPG | |
| | Light Chain | DIQMTQSPSSLSASVGDRVTITC | 42 |
| | | KASQDISNYLSWFQQKPGKAPKL | |
| | | LIYRANRLVEGVPSRFSGSGSGT | |
| | | DFTLTISSLQPEDFATYFCLQYD | |
| | | EFPLTFG | |
| | | GGTKVEIKRTVAAPSVFIFPPSD | |
| | | EQLKSGTASVVCLLNNFYPREAK | |
| | | VQWKVDNALQSGNSQESVTEQDS | |
| | | KDSTYSLSSTLTLSKADYEKHKV | |
| | | YACEVTHQGLSSPVTKSFNRGEC | |
| B2 | HC CDR1 | GFNIKDYY | 13 |
| | HC CDR2 | IDPEDAES | 43 |
| | HC CDR3 | TRGDSMMVTYFDY | 15 |
| | LC CDR1 | QDVSTA | 16 |
| | LC CDR2 | WAF | N/A |
| | LC CDR3 | QQHYRSPWT | 18 |
| | VH | EVQLVQSGAEVKKPGASVKVSCK | 44 |
| | | ASGFNIKDYYIHWVRQATGQGLE | |
| | | WMGRIDPEDAESEYAPKFQGRVT | |
| | | ITADTSTDTAYMELSSLRSEDTA | |
| | | VYYCTRGDSMMVTYFDYWGQGTL | |
| | | VTVSS | |
| | VL | DIQMTQSPSSLSASVGDRVTITC | 45 |
| | | KASQDVSTAVAWYQQKPGKAPKL | |
| | | LIYWAFTRHTGVPSRFSGSGSGT | |
| | | DYALTISSLQPEDFATYYCQQHY | |
| | | RSPWTFGGGTKVEIK | |
| | HC Constant | ASTKGPSVFPLAPSSKSTSGGTA | 39 |
| | Region | ALGCLVKDYFPEPVTVSWNSGAL | |
| | | TSGVHTFPAVLQSSGLYSLSSVV | |
| | | TVPSSSLGTQTYICNVNHKPSNT | |
| | | KVDKKVEPKSCDKTHTCPPCPAP | |
| | | ELLGGPSVFLFPPKPKDTLMISR | |
| | | TPEVTCVVVDVSHEDPEVKFNWY | |
| | | VDGVEVHNAKTKPREEQYASTYR | |
| | | VVSVLTVLHQDWLNGKEYKCKVS | |
| | | NKALPAPIEKTISKAKGQPREPQ | |
| | | VYTLPPSRDELTKNQVSLTCLVK | |
| | | GFYPSDIAVEWESNGQPENNYKT | |
| | | TPPVLDSDGSFFLYSKLTVDKSR | |
| | | WQQGNVFSCSVMHEALHNHYTQK | |
| | | SLSLSPG | |
| | LC Constant | RTVAAPSVFIFPPSDEQLKSGTA | 40 |
| | Region | SVVCLLNNFYPREAKVQWKVDNA | |
| | | LQSGNSQESVTEQDSKDSTYSLS | |
| | | STLTLSKADYEKHKVYACEVTHQ | |
| | | GLSSPVTKSFNRGEC | |
| | Heavy Chain | EVQLVQSGAEVKKPGASVKVSCK | 46 |
| | | ASGFNIKDYYIHWVRQATGQGLE | |
| | | WMGRIDPEDAESEYAPKFQGRVT | |
| | | ITADTSTDTAYMELSSLRSEDTA | |
| | | VYYCTRGDSMMVTYFDYWGQGTL | |
| | | VTVSSASTKGPSVFPLAPSSKST | |
| | | SGGTAALGCLVKDYFPEPVTVSW | |
| | | NSGALTSGVHTFPAVLQSSGLYS | |
| | | LSSVVTVPSSSLGTQTYICNVNH | |
| | | KPSNTKVDKKVEPKSCDKTHTCP | |
| | | PCPAPELLGGPSVFLFPPKPKDT | |
| | | LMISRTPEVTCVVVDVSHEDPEV | |
| | | KFNWYVDGVEVHNAKTKPREEQY | |
| | | ASTYRVVSVLTVLHQDWLNGKEY | |
| | | KCKVSNKALPAPIEKTISKAKGQ | |
| | | PREPQVYTLPPSRDELTKNQVSL | |
| | | TCLVKGFYPSDIAVEWESNGQPE | |
| | | NNYKTTPPVLDSDGSFFLYSKLT | |
| | | VDKSRWQQGNVFSCSVMHEALHN | |
| | | HYTQKSLSLSPG | |
| | Light Chain | DIQMTQSPSSLSASVGDRVTITC | 47 |
| | | KASQDVSTAVAWYQQKPGKAPKL | |
| | | LIYWAFTRHTGVPSRFSGSGSGT | |
| | | DYALTISSLQPEDFATYYCQQHY | |
| | | RSPWTFGGGTKVEIKRTVAAPSV | |
| | | FIFPPSDEQLKSGTASVVCLLNN | |
| | | FYPREAKVQWKVDNALQSGNSQE | |
| | | SVTEQDSKDSTYSLSSTLTLSKA | |
| | | DYEKHKVYACEVTHQGLSSPVTK | |
| | | SFNRGEC | |
| C3 | HC CDR1 | GFNIEDYY | 24 |
| | HC CDR2 | IDPEDAET | 48 |
| | HC CDR3 | ARSIYLDPMDY | 26 |
| | LC CDR1 | QDVTTA | 27 |
| | LC CDR2 | WAT | N/A |
| | LC CDR3 | QQHYSTPYT | 29 |
| | VH | QVQLVQSGAEVKKPGASVKVSCK | 49 |
| | | ASGFNIEDYYMHWVRQAPGQRLE | |
| | | WMGRIDPEDAETTYSPKFQGRVT | |
| | | IIPDTSANTAYMELSSLRSEDTA | |
| | | VYYCARSIYLDPMDYWGQGTLVT | |
| | | VSS | |
| | VL | DIQMTQSPKSLSASVGDRVTITC | 50 |
| | | RASQDVTTALAWYQQKPGQSPKL | |
| | | LIYWATTRHSGVPSRFSGSGSGT | |
| | | DFTLTISSLQPEDFATYYCQQHY | |
| | | STPYTFGQGTKLEIK | |
| | HC Constant | ASTKGPSVFPLAPSSKSTSGGTA | 51 |
| | Region | ALGCLVKDYFPEPVTVSWNSGAL | |
| | | TSGVHTFPAVLQSSGLYSLSSVV | |
| | | TVPSSSLGTQTYICNVNHKPSNT | |
| | | KVDKKVEPKSCDKTHTCPPCPAP | |
| | | EAAGGPSVFLFPPKPKDTLMISR | |
| | | TPEVTCVVVDVSHEDPEVKFNWY | |
| | | VDGVEVHNAKTKPREEQYNSTYR | |
| | | VVSVLTVLHQDWLNGKEYKCKVS | |
| | | NKALPAPIEKTISKAKGQPREPQ | |
| | | VYTLPPSREEMTKNQVSLTCLVK | |
| | | GFYPSDIAVEWESNGQPENNYKT | |
| | | TPPVLDSDGSFFLYSKLTVDKSR | |
| | | WQQGNVFSCSVMHEALHNHYTQK | |
| | | SLSLSPG | |
| | LC Constant | RTVAAPSVFIFPPSDEQLKSGTA | 40 |
| | Region | SVVCLLNNFYPREAKVQWKVDNA | |
| | | LQSGNSQESVTEQDSKDSTYSLS | |
| | | STLTLSKADYEKHKVYACEVTHQ | |
| | | GLSSPVTKSFNRGEC | |
| | Heavy Chain | QVQLVQSGAEVKKPGASVKVSCK | 52 |
| | | ASGFNIEDYYMHWVRQAPGQRLE | |
| | | WMGRIDPEDAETTYSPKFQGRVT | |
| | | IIPDTSANTAYMELSSLRSEDTA | |
| | | VYYCARSIYLDPMDYWGQGTLVT | |
| | | VSSASTKGPSVFPLAPSSKSTSG | |
| | | GTAALGCLVKDYFPEPVTVSWNS | |
| | | GALTSGVHTFPAVLQSSGLYSLS | |
| | | SVVTVPSSSLGTQTYICNVNHKP | |
| | | SNTKVDKKVEPKSCDKTHTCPPC | |
| | | PAPEAAGGPSVFLFPPKPKDTLM | |
| | | ISRTPEVTCVVVDVSHEDPEVKF | |
| | | NWYVDGVEVHNAKTKPREEQYNS | |
| | | TYRVVSVLTVLHQDWLNGKEYKC | |
| | | KVSNKALPAPIEKTISKAKGQPR | |
| | | EPQVYTLPPSREEMTKNQVSLTC | |
| | | LVKGFYPSDIAVEWESNGQPENN | |
| | | YKTTPPVLDSDGSFFLYSKLTVD | |
| | | KSRWQQGNVFSCSVMHEALHNHY | |
| | | TQKSLSLSPG | |
| | Light Chain | DIQMTQSPKSLSASVGDRVTITC | 53 |
| | | RASQDVTTALAWYQQKPGQSPKL | |
| | | LIYWATTRHSGVPSRFSGSGSGT | |
| | | DFTLTISSLQPEDFATYYCQQHY | |
| | | STPYTFGQGTKLEIKRTVAAPSV | |
| | | FIFPPSDEQLKSGTASVVCLLNN | |
| | | FYPREAKVQWKVDNALQSGNSQE | |
| | | SVTEQDSKDSTYSLSSTLTLSKA | |
| | | DYEKHKVYACEVTHQGLSSPVTK | |
| | | SFNRGEC | |

In some embodiments, the anti-TMPRSS6 antibodies of the present disclosure comprise one or more of the HC CDRs (e.g., HC CDR1, HC CDR2, or HC CDR3) amino acid sequences from any one of the anti-TMPRSS6 antibodies selected from Table 1. In some embodiments, the anti-TMPRSS6 antibodies of the present disclosure comprise the HC CDR1, HC CDR2, and HC CDR3 as provided for any one of the antibodies elected from Table 1. In some embodiments, the anti-TMPRSS6 antibodies of the present disclosure comprise one or more of the LC CDRs (e.g., LC CDR1, LC CDR2, or LC CDR3) amino acid sequences from any one of the anti-TMPRSS6 antibodies selected from Table 1. In some embodiments, the anti-TMPRSS6 antibodies of the present disclosure comprise the LC CDR1, LC CDR2, and LC CDR3 s provided for any one of the anti-TMPRSS6 antibodies selected from Table 1.

In some embodiments, the anti-TMPRSS6 antibodies of the present disclosure comprises the HC CDR1, HC CDR2, HC CDR3, LC CDR1, LC CDR2, and LC CDR3 as provided for any one of the anti-TMPRSS6 antibodies selected from Table 1. In some embodiments, antibody heavy and light chain CDR3 domains may play a particularly important role in the binding specificity/affinity of an antibody for an antigen. Accordingly, the anti-TMPRSS6 antibodies of the disclosure may include at least the heavy and/or light chain CDR3s of any one of the anti-TMPRSS6 antibodies selected from Table 1.

In some embodiments, the anti-TMPRSS6 antibody comprises a heavy chain variable region that comprises a heavy chain CDR1 (HC CDR1), a heavy chain CDR2 (HC CDR2), and a heavy chain CDR3 (HC CDR3). In some embodiments, the anti-TMPRSS6 antibody comprises a light chain variable region that comprises a light chain CDR1 (LC CDR1), a light chain CDR2 (LC CDR2), and a light chain CDR3 (LC CDR3).

Also within the scope of the present disclosure are functional variants of any of the exemplary anti-TMPRSS6 antibodies as disclosed herein. A functional variant may contain one or more amino acid residue variations in the VH and/or VL, or in one or more of the HC CDRs and/or one or more of the LC CDRs as relative to the reference antibody, while retaining substantially similar binding and biological activities (e.g., substantially similar binding affinity, binding specificity, inhibitory activity, or a combination thereof) as the reference antibody.

In some embodiments, any of the anti-TMPRSS6 antibodies of the disclosure have one or more CDRs (e.g., HC CDR or LC CDR) sequences substantially similar to any of the HC CDR1, HC CDR2, HC CDR3, LC CDR1, LC CDR2, and/or LC CDR3 sequences from one of the anti-TMPRSS6 antibodies selected from Table 1. In some embodiments, the position of one or more CDRs along the VH (e.g., HC CDR1, HC CDR2, or HC CDR3) and/or VL (e.g., LC CDR1, LC CDR2, or LC CDR3) region of an antibody described herein can vary by one, two, three, four, five, or six amino acid positions so long as immunospecific binding to TMPRSS6 (e.g., human TMPRSS6) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or C-terminal boundary of the CDR by one, two, three, four, five, or six amino acids, relative to the CDR position of any one of the antibodies described herein, so long as immunospecific binding to TMPRSS6 (e.g., human TMPRSS6) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., HC CDR1, HC CDR2, or HC CDR3) and/or VL (e.g., LC CDR1, LC CDR2, or LC CDR3) region of an antibody described herein can vary (e.g., be shorter or longer) by one, two, three, four, five, or more amino acids, so long as immunospecific binding to TMPRSS6 (e.g., human TMPRSS6) is maintained (e.g., substantially maintained, for example, at least 50%, at least 60%, at least 70%, at least 80%, at least 90%, at least 95% of the binding of the original antibody from which it is derived).

Accordingly, in some embodiments, a HC CDR1, HC CDR2, HC CDR3, LC CDR1, LC CDR2, and/or LC CDR3 described herein may be one, two, three, four, five or more amino acids shorter than one or more of the CDRs described herein (e.g., CDRs from any of the anti-TMPRSS6 antibodies selected from Table 1) so long as immunospecific binding to TMPRSS6 (e.g., human TMPRSS6) is maintained (e.g., substantially maintained, for example, at least 50%, at least 60%, at least 70%, at least 80%, at least 90%, at least 95% relative to the binding of the original antibody from which it is derived). In some embodiments, a HC CDR1, HC CDR2, HC CDR3, LC CDR1, LC CDR2, and/or LC CDR3 described herein may be one, two, three, four, five or more amino acids longer than one or more of the CDRs described herein (e.g., CDRs from any of the anti-TMPRSS6 antibodies selected from Table 1) so long as immunospecific binding to TMPRSS6 (e.g., human TMPRSS6) is maintained (e.g., substantially maintained, for example, at least 50%, at least 60%, at least 70%, at least 80%, at least 90%, at least 95% relative to the binding of the original antibody from which it is derived). In some embodiments, the amino portion of a HC CDR1, HC CDR2, HC CDR3, LC CDR1, LC CDR2, and/or LC CDR3 described herein can be extended by one, two, three, four, five or more amino acids compared to one or more of the CDRs described herein (e.g., CDRs from any of the anti-TMPRSS6 antibodies selected from Table 1) so long as immunospecific binding to TMPRSS6 (e.g., human TMPRSS6) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a HC CDR1, HC CDR2, HC CDR3, LC CDR1, LC CDR2, and/or LC CDR3 described herein can be extended by one, two, three, four, five or more amino acids compared to one or more of the CDRs described herein (e.g., CDRs from any of the anti-TMPRSS6 antibodies selected from Table 1) so long as immunospecific binding to TMPRSS6 (e.g., human TMPRSS6) is maintained (e.g., substantially maintained, for example, at least 50%, at least 60%, at least 70%, at least 80%, at least 90%, at least 95% relative to the binding of the original antibody from which it is derived). In some embodiments, the amino portion of a HC CDR1, HC CDR2, HC CDR3, LC CDR1, LC CDR2, and/or LC CDR3 described herein can be shortened by one, two, three, four, five or more amino acids compared to one or more of the CDRs described herein (e.g., CDRs from any of the anti-TMPRSS6 antibodies selected from Table 1) so long as immunospecific binding to TMPRSS6 (e.g., human TMPRSS6) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a HC CDR1, HC CDR2, HC CDR3, LC CDR1, LC CDR2, and/or LC CDR3 described herein can be shortened by one, two, three, four, five or more amino acids compared to one or more of the CDRs described herein (e.g., CDRs from any of the anti-TMPRSS6 antibodies selected from Table 1) so long as immunospecific binding to TMPRSS6 (e.g., human TMPRSS6) is maintained (e.g., substantially maintained, for example, at least 50%, at least 60%, at least 70%, at least 80%, at least 90%, at least 95% relative to the binding of the original antibody from which it is derived). Any method can be used to ascertain whether immunospecific binding to TMPRSS6 (e.g., human TMPRSS6) is maintained, for example, using binding assays and conditions described in the art.

In some examples, any of the anti-TMPRSS6 antibodies of the disclosure have one or more CDR (e.g., HC CDR or LC CDR) sequences substantially similar to any one of the anti-TMPRSS6 antibodies selected from Table 1. For example, the antibodies may include one or more CDR sequence(s) from any of the anti-TMPRSS6 antibodies selected from Table 1 containing up to 5, 4, 3, 2, or 1 amino acid residue variations as compared to the corresponding CDR region in any one of the CDRs provided herein (e.g., CDRs from any of the anti-TMPRSS6 antibodies selected from Table 1) so long as immunospecific binding to TMPRSS6 (e.g., human TMPRSS6) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a TMPRSS6 protein (e.g., a human TMPRSS6 protein), for example, as determined based on a crystal structure. Some aspects of the disclosure provide anti-TMPRSS6 antibodies that comprise one or more of the heavy chain variable (VH) and/or light chain variable (VL) domains provided herein. In some embodiments, any of the VH domains provided herein include one or more of the HC CDR sequences (e.g., HC CDR1, HC CDR2, and HC CDR3) provided herein, for example, any of the CDR-H sequences provided in any one of the anti-TMPRSS6 selected from Table 1. In some embodiments, any of the VL domains provided herein include one or more of the CDR-L sequences (e.g., LC CDR1, LC CDR2, and LC CDR3) provided herein, for example, any of the LC CDR sequences provided in any one of the anti-TMPRSS6 antibodies selected from Table 1.

In some embodiments, an anti-TMPRSS6 antibody of the disclosure include any antibody that includes a heavy chain variable domain and/or a light chain variable domain of any one of the anti-TMPRSS6 antibodies selected from Table 1, and variants thereof. In some embodiments, anti-TMPRSS6 antibodies of the disclosure include any antibody that includes the heavy chain variable and light chain variable pairs of any anti-TMPRSS6 antibodies selected from Table 1.

Aspects of the disclosure provide anti-TMPRSS6 antibodies having a heavy chain variable (VH) and/or a light chain variable (VL) domain amino acid sequence homologous to any of those described herein. In some embodiments, the anti-TMPRSS6 antibody comprises a heavy chain variable sequence or a light chain variable sequence that is at least 75% (e.g., at least 80%, at least 85%, at least 90%, at least 91%, at least 92%, at least 93%, at least 94%, at least 95%, at least 96%, at least 97%, at least 98%, or at least 99%) identical to the heavy chain variable sequence and/or any light chain variable sequence of any one of the anti-TMPRSS6 antibodies selected from Table 1. In some embodiments, a heavy chain variable and/or a light chain variable amino acid sequences do not vary within any of the CDR sequences provided herein. For example, in some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a heavy chain variable and/or a light chain variable sequence excluding any of the CDR sequences provided herein. In some embodiments, any of the anti-TMPRSS6 antibodies provided herein comprise a heavy chain variable sequence and a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of any anti-TMPRSS6 antibodies selected from Table 1.

In some embodiments, an anti-TMPRSS6 antibody of the present disclosure is an antibody comprising a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-TMPRSS6 antibodies listed in Table 1. Alternatively or in addition, the anti-TMPRSS6 antibody of the present disclosure is a humanized antibody comprising a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL of any one of the anti-TMPRSS6 antibodies listed in Table 1. In some embodiments, a heavy chain variable and/or a light chain variable amino acid sequences do not vary within any of the CDR sequences provided herein. For example, in some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a heavy chain variable and/or a light chain variable sequence excluding any of the CDR sequences provided herein. In some embodiments, any of the anti-TMPRSS6 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of any one of anti-TMPRSS6 antibodies selected from Table 1 and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of any one of anti-TMPRSS6 antibodies selected from Table 1.

In some embodiments, an anti-TMPRSS6 antibody of the present disclosure is a humanized antibody (e.g., a humanized variant containing one or more CDRs of Table 1). In some embodiments, an anti-TMPRSS6 antibody of the present disclosure comprises a HC CDR1, a HC CDR2, a HC CDR3, a LC CDR1, a LC CDR2, and a LC CDR3 that are the same as the HC CDR1, HC CDR2, HC CDR3, LC CDR1, LC CDR2, and LC CDR3 shown in Table 1, and comprises a humanized heavy chain variable region and/or a humanized light chain variable region. In some embodiments, a humanized heavy chain variable and/or a humanized light chain variable amino acid sequences do not vary within any of the CDR sequences provided herein. For example, in some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a heavy chain variable and/or a light chain variable sequence excluding any of the CDR sequences provided herein. In some embodiments, a humanized anti-TMPRSS6 antibody provided herein comprises a heavy chain variable sequence and a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of any anti-TMPRSS6 antibodies selected from Table 1. In some embodiments, a humanized heavy chain variable and/or a humanized light chain variable amino acid sequences do not vary within any of the CDR sequences provided herein. For example, in some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a heavy chain variable and/or a light chain variable sequence excluding any of the CDR sequences provided herein. In some embodiments, a humanized anti-TMPRSS6 antibody provided herein comprises a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of any one of anti-TMPRSS6 antibodies selected from Table 1 and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of any one of anti-TMPRSS6 antibodies selected from Table 1.

In some embodiments, an anti-TMPRSS6 antibody of the present disclosure comprises a HC CDR1, HC CDR2 and HC CDR3 of a heavy chain variable domain having the amino acid sequence of SEQ ID NO: 7. Alternatively or in addition, the anti-TMPRSS6 antibody of the present disclosure comprises a LC CDR1, LC CDR2 and LC CDR3 of a light chain variable domain having the amino acid sequence of SEQ ID NO: 8.

In some embodiments, according to the Kabat definition system, an anti-TMPRSS6 antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 4, a LC CDR2 having the amino acid sequence of RAN, and a LC CDR3 having the amino acid sequence of SEQ ID NO: 6.

In some embodiments, an anti-TMPRSS6 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-TMPRSS6 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of RAN, and LC CDR3 having the amino acid sequence of SEQ ID NO: 6.

In some embodiments, an anti-TMPRSS6 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-TMPRSS6 antibody of the present disclosure at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 4, LC CDR2 having the amino acid sequence of RAN, and LC CDR3 having the amino acid sequence of SEQ ID NO: 6.

In some embodiments, an anti-TMPRSS6 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-TMPRSS6 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of RAN; and/or a LC CDR3 having no more than 3 amino acid variations (e.g., no more than 3, 2, or 1 amino acid variation) as compared with the LC CDR3 having the amino acid sequence of SEQ ID NO: 6.

In some embodiments, an anti-TMPRSS6 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 2; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 3. Alternatively or in addition, the anti-TMPRSS6 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 4; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of RAN; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 6.

In some embodiments, an anti-TMPRSS6 antibody of the present disclosure comprises a VH comprising the amino acid sequence of SEQ ID NO: 7. Alternatively or in addition, the anti-TMPRSS6 antibody of the present disclosure comprises a VL comprising the amino acid sequence of SEQ ID NO: 8.

In some embodiments, an anti-TMPRSS6 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, the anti-TMPRSS6 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 8. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 7 and/or a VL of SEQ ID NO: 8 excluding any of the CDR sequences therein. In some embodiments, an anti-TMPRSS6 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 7, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 8.

In some embodiments, an anti-TMPRSS6 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 7. Alternatively or in addition, the anti-TMPRSS6 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 8. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 7, and/or a VL of SEQ ID NO: 8 excluding any of the CDR sequences therein. In some embodiments, an anti-TMPRSS6 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 7, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 8.

In some embodiments, an anti-TMPRSS6 antibody of the present disclosure comprises a HC CDR1, HC CDR2 and HC CDR3 of a heavy chain variable domain having the amino acid sequence of SEQ ID NO: 19 or 78. Alternatively or in addition, the anti-TMPRSS6 antibody of the present disclosure comprises a LC CDR1, LC CDR2 and LC CDR3 of a light chain variable domain having the amino acid sequence of SEQ ID NO: 20.

In some embodiments, according to the Kabat definition system, an anti-TMPRSS6 antibody of the present disclosure comprises a HC CDR1 having the amino acid sequence of SEQ ID NO: 13, a HC CDR2 having the amino acid sequence of SEQ ID NO: 14, a HC CDR3 having the amino acid sequence of SEQ ID NO: 15, a LC CDR1 having the amino acid sequence of SEQ ID NO: 16, a LC CDR2 having the amino acid sequence of WAF, and a LC CDR3 having the amino acid sequence of SEQ ID NO: 18.

In some embodiments, an anti-TMPRSS6 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 13, HC CDR2 having the amino acid sequence of SEQ ID NO: 14, and HC CDR3 having the amino acid sequence of SEQ ID NO: 15. Alternatively or in addition, the anti-TMPRSS6 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 16, LC CDR2 having the amino acid sequence of WAF, and LC CDR3 having the amino acid sequence of SEQ ID NO: 18.

In some embodiments, an anti-TMPRSS6 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 13, HC CDR2 having the amino acid sequence of SEQ ID NO: 14, and HC CDR3 having the amino acid sequence of SEQ ID NO: 15. Alternatively or in addition, the anti-TMPRSS6 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 16, LC CDR2 having the amino acid sequence of WAF, and LC CDR3 having the amino acid sequence of SEQ ID NO: 18.

In some embodiments, an anti-TMPRSS6 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 13; a HC CDR2 having no more than 3 amino acid variations (e.g., no more than 3, 2, or 1 amino acid variation) as compared with the HC CDR2 having the amino acid sequence of SEQ ID NO: 14; and/or a HC CDR3 having no more than 3 amino acid variations (e.g., no more than 3, 2, or 1 amino acid variation) as compared with the HC CDR3 having the amino acid sequence of SEQ ID NO: 15. Alternatively or in addition, the anti-TMPRSS6 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 16; a LC CDR2 having no more than 3 amino acid variations (e.g., no more than 3, 2, or 1 amino acid variation) as compared with the LC CDR2 having the amino acid sequence of WAF; and/or a LC CDR3 having no more than 3 amino acid variations (e.g., no more than 3, 2, or 1 amino acid variation) as compared with the LC CDR3 having the amino acid sequence of SEQ ID NO: 18.

In some embodiments, an anti-TMPRSS6 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 13; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 14; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 15. Alternatively or in addition, the anti-TMPRSS6 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 16; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of WAF; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 18.

In some embodiments, an anti-TMPRSS6 antibody of the present disclosure comprises a VH comprising the amino acid sequence of SEQ ID NO: 19 or 78. Alternatively or in addition, the anti-TMPRSS6 antibody of the present disclosure comprises a VL comprising the amino acid sequence of SEQ ID NO: 20.

In some embodiments, an anti-TMPRSS6 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 19 or 78. Alternatively or in addition, the anti-TMPRSS6 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 20. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 19 or 78, and/or a VL of SEQ ID NO: 20 excluding any of the CDR sequences therein. In some embodiments, an anti-TMPRSS6 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 19 or 78, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 20.

In some embodiments, an anti-TMPRSS6 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 19 or 78. Alternatively or in addition, the anti-TMPRSS6 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 20. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 19 or 78, and/or a VL of SEQ ID NO: 20 excluding any of the CDR sequences therein. In some embodiments, an anti-TMPRSS6 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 19 or 78, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 20.

In some embodiments, an anti-TMPRSS6 antibody of the present disclosure comprises a HC CDR1, HC CDR2 and HC CDR3 of a heavy chain variable domain having the amino acid sequence of SEQ ID NO: 30. Alternatively, or in addition, the anti-TMPRSS6 antibody of the present disclosure comprises a LC CDR1, LC CDR2 and LC CDR3 of a light chain variable domain having the amino acid sequence of SEQ ID NO: 31.

In some embodiments, according to the Kabat definition system, an anti-TMPRSS6 antibody of the present disclosure comprises a HC CDR1 having the amino acid sequence of SEQ ID NO: 24, a HC CDR2 having the amino acid sequence of SEQ ID NO: 25, a HC CDR3 having the amino acid sequence of SEQ ID NO: 26, a LC CDR1 having the amino acid sequence of SEQ ID NO: 27, a LC CDR2 having the amino acid sequence of WAT, and a LC CDR3 having the amino acid sequence of SEQ ID NO: 29.

In some embodiments, an anti-TMPRSS6 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 24, HC CDR2 having the amino acid sequence of SEQ ID NO: 25, and HC CDR3 having the amino acid sequence of SEQ ID NO: 26. Alternatively or in addition, the anti-TMPRSS6 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 27, LC CDR2 having the amino acid sequence of WAT, and LC CDR3 having the amino acid sequence of SEQ ID NO: 29.

In some embodiments, an anti-TMPRSS6 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 24, HC CDR2 having the amino acid sequence of SEQ ID NO: 25, and HC CDR3 having the amino acid sequence of SEQ ID NO: 26. Alternatively or in addition, the anti-TMPRSS6 antibody of the present disclosure at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 27, LC CDR2 having the amino acid sequence of WAT, and LC CDR3 having the amino acid sequence of SEQ ID NO: 29.

In some embodiments, an anti-TMPRSS6 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 24; a HC CDR2 having no more than 3 amino acid variations (e.g., no more than 3, 2, or 1 amino acid variation) as compared with the HC CDR2 having the amino acid sequence of SEQ ID NO: 25; and/or a HC CDR3 having no more than 3 amino acid variations (e.g., no more than 3, 2, or 1 amino acid variation) as compared with the HC CDR3 having the amino acid sequence of SEQ ID NO: 26. Alternatively or in addition, the anti-TMPRSS6 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 27; a LC CDR2 having no more than 3 amino acid variations (e.g., no more than 3, 2, or 1 amino acid variation) as compared with the LC CDR2 having the amino acid sequence of WAT; and/or a LC CDR3 having no more than 3 amino acid variations (e.g., no more than 3, 2, or 1 amino acid variation) as compared with the LC CDR3 having the amino acid sequence of SEQ ID NO: 29.

In some embodiments, an anti-TMPRSS6 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 24; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 25; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 26. Alternatively or in addition, the anti-TMPRSS6 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 27; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of WAT; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 29.

In some embodiments, an anti-TMPRSS6 antibody of the present disclosure comprises a VH comprising the amino acid sequence of SEQ ID NO: 30. Alternatively, or in addition, the anti-TMPRSS6 antibody of the present disclosure comprises a VL comprising the amino acid sequence of SEQ ID NO: 31.

In some embodiments, an anti-TMPRSS6 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 30. Alternatively, or in addition, the anti-TMPRSS6 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 31. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 30 and/or a VL of SEQ ID NO: 31 excluding any of the CDR sequences therein. In some embodiments, an anti-TMPRSS6 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 30, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 31.

In some embodiments, an anti-TMPRSS6 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 30. Alternatively, or in addition, the anti-TMPRSS6 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 31. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 30, and/or a VL of SEQ ID NO: 31 excluding any of the CDR sequences therein. In some embodiments, an anti-TMPRSS6 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 30, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 31.

In some embodiments, an anti-TMPRSS6 antibody of the present disclosure comprises a HC CDR1, HC CDR2 and HC CDR3 of a heavy chain variable domain having the amino acid sequence of SEQ ID NO: 37. Alternatively, or in addition, the anti-TMPRSS6 antibody of the present disclosure comprises a LC CDR1, LC CDR2 and LC CDR3 of a light chain variable domain having the amino acid sequence of SEQ ID NO: 38.

In some embodiments, according to the Kabat definition system, the anti-TMPRSS6 antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 35, a LC CDR1 having the amino acid sequence of SEQ ID NO: 36, a LC CDR2 having the amino acid sequence of RAN, and a LC CDR3 having the amino acid sequence of SEQ ID NO: 6.

In some embodiments, an anti-TMPRSS6 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 35. Alternatively or in addition, an anti-TMPRSS6 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 36, LC CDR2 having the amino acid sequence of RAN, and LC CDR3 having the amino acid sequence of SEQ ID NO: 6.

In some embodiments, an anti-TMPRSS6 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 35. Alternatively or in addition, the anti-TMPRSS6 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 36, LC CDR2 having the amino acid sequence of RAN, and LC CDR3 having the amino acid sequence of SEQ ID NO: 6.

In some embodiments, an anti-TMPRSS6 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 35. Alternatively or in addition, the anti-TMPRSS6 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 36; a LC CDR2 having no more than 3 amino acid variations (e.g., no more than 3, 2, or 1 amino acid variation) as compared with the LC CDR2 having the amino acid sequence of RAN; and/or a LC CDR3 having no more than 3 amino acid variations (e.g., no more than 3, 2, or 1 amino acid variation) as compared with the LC CDR3 having the amino acid sequence of SEQ ID NO: 6.

In some embodiments, an anti-TMPRSS6 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 2; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 35. Alternatively or in addition, the anti-TMPRSS6 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 36; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of RAN; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 6.

In some embodiments, an anti-TMPRSS6 antibody of the present disclosure comprises a VH comprising the amino acid sequence of SEQ ID NO: 37. Alternatively, or in addition, the anti-TMPRSS6 antibody of the present disclosure comprises a VL comprising the amino acid sequence of SEQ ID NO: 38.

In some embodiments, an anti-TMPRSS6 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 37. Alternatively, or in addition, the anti-TMPRSS6 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 38. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 37 and/or a VL of SEQ ID NO: 38 excluding any of the CDR sequences therein. In some embodiments, an anti-TMPRSS6 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 37, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 38.

In some embodiments, an anti-TMPRSS6 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 37. Alternatively, or in addition, the anti-TMPRSS6 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) to the VL as set forth in SEQ ID NO: 38. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 37, and/or a VL of SEQ ID NO: 38 excluding any of the CDR sequences therein. In some embodiments, an anti-TMPRSS6 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 37, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 38.

In some embodiments, an anti-TMPRSS6 antibody of the present disclosure comprises a HC CDR1, HC CDR2 and HC CDR3 of a heavy chain variable domain having the amino acid sequence of SEQ ID NO: 44. Alternatively, or in addition, the anti-TMPRSS6 antibody of the present disclosure comprises a LC CDR1, LC CDR2 and LC CDR3 of a light chain variable domain having the amino acid sequence of SEQ ID NO: 45.

In some embodiments, according to the Kabat definition system, an anti-TMPRSS6 antibody of the present disclosure comprises a HC CDR1 having the amino acid sequence of SEQ ID NO: 13, a HC CDR2 having the amino acid sequence of SEQ ID NO: 43, a HC CDR3 having the amino acid sequence of SEQ ID NO: 15, a LC CDR1 having the amino acid sequence of SEQ ID NO: 16, a LC CDR2 having the amino acid sequence of WAF, and a LC CDR3 having the amino acid sequence of SEQ ID NO: 18.

In some embodiments, an anti-TMPRSS6 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 13, HC CDR2 having the amino acid sequence of SEQ ID NO: 43, and HC CDR3 having the amino acid sequence of SEQ ID NO: 15. Alternatively or in addition, an anti-TMPRSS6 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 16, LC CDR2 having the amino acid sequence of WAF, and LC CDR3 having the amino acid sequence of SEQ ID NO: 18.

In some embodiments, an anti-TMPRSS6 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 13, HC CDR2 having the amino acid sequence of SEQ ID NO: 43, and HC CDR3 having the amino acid sequence of SEQ ID NO: 15. Alternatively or in addition, the anti-TMPRSS6 antibody of the present disclosure at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 16, LC CDR2 having the amino acid sequence of WAF, and LC CDR3 having the amino acid sequence of SEQ ID NO: 18.

In some embodiments, an anti-TMPRSS6 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 13; a HC CDR2 having no more than 3 amino acid variations (e.g., no more than 3, 2, or 1 amino acid variation) as compared with the HC CDR2 having the amino acid sequence of SEQ ID NO: 43; and/or a HC CDR3 having no more than 3 amino acid variations (e.g., no more than 3, 2, or 1 amino acid variation) as compared with the HC CDR3 having the amino acid sequence of SEQ ID NO: 15. Alternatively or in addition, the anti-TMPRSS6 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 16; a LC CDR2 having no more than 3 amino acid variations (e.g., no more than 3, 2, or 1 amino acid variation) as compared with the LC CDR2 having the amino acid sequence of WAF; and/or a LC CDR3 having no more than 3 amino acid variations (e.g., no more than 3, 2, or 1 amino acid variation) as compared with the LC CDR3 having the amino acid sequence of SEQ ID NO: 18.

In some embodiments, an anti-TMPRSS6 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 13; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 43; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 15. Alternatively or in addition, the anti-TMPRSS6 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 16; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of WAF; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 18.

In some embodiments, an anti-TMPRSS6 antibody of the present disclosure comprises a VH comprising the amino acid sequence of SEQ ID NO: 44. Alternatively, or in addition, the anti-TMPRSS6 antibody of the present disclosure comprises a VL comprising the amino acid sequence of SEQ ID NO: 45.

In some embodiments, an anti-TMPRSS6 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 44. Alternatively, or in addition, the anti-TMPRSS6 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 45. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 44 and/or a VL of SEQ ID NO: 45 excluding any of the CDR sequences therein. In some embodiments, an anti-TMPRSS6 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 44, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 45.

In some embodiments, an anti-TMPRSS6 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) to the VH as set forth in SEQ ID NO: 44. Alternatively, or in addition, the anti-TMPRSS6 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 45. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 44, and/or a VL of SEQ ID NO: 45 excluding any of the CDR sequences therein. In some embodiments, an anti-TMPRSS6 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 44, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 45.

In some embodiments, an anti-TMPRSS6 antibody of the present disclosure comprises a HC CDR1, HC CDR2 and HC CDR3 of a heavy chain variable domain having the amino acid sequence of SEQ ID NO: 49. Alternatively, or in addition, the anti-TMPRSS6 antibody of the present disclosure comprises a LC CDR1, LC CDR2 and LC CDR3 of a light chain variable domain having the amino acid sequence of SEQ ID NO: 50.

In some embodiments, according to the Kabat definition system, an anti-TMPRSS6 antibody of the present disclosure comprises a HC CDR1 having the amino acid sequence of SEQ ID NO: 24, a HC CDR2 having the amino acid sequence of SEQ ID NO: 48, a HC CDR3 having the amino acid sequence of SEQ ID NO: 26, a LC CDR1 having the amino acid sequence of SEQ ID NO: 27, a LC CDR2 having the amino acid sequence of WAT, and a LC CDR3 having the amino acid sequence of SEQ ID NO: 29.

In some embodiments, an anti-TMPRSS6 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 24, HC CDR2 having the amino acid sequence of SEQ ID NO: 48, and HC CDR3 having the amino acid sequence of SEQ ID NO: 26. Alternatively or in addition, the anti-TMPRSS6 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 27, LC CDR2 having the amino acid sequence of WAT, and LC CDR3 having the amino acid sequence of SEQ ID NO: 29.

In some embodiments, an anti-TMPRSS6 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) to the HC CDR1 having the amino acid sequence of SEQ ID NO: 24, HC CDR2 having the amino acid sequence of SEQ ID NO: 48, and HC CDR3 having the amino acid sequence of SEQ ID NO: 26. Alternatively or in addition, the anti-TMPRSS6 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 27, LC CDR2 having the amino acid sequence of WAT, and LC CDR3 having the amino acid sequence of SEQ ID NO: 29.

In some embodiments, an anti-TMPRSS6 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 24; a HC CDR2 having no more than 3 amino acid variations (e.g., no more than 3, 2, or 1 amino acid variation) as compared with the HC CDR2 having the amino acid sequence of SEQ ID NO: 48; and/or a HC CDR3 having no more than 3 amino acid variations (e.g., no more than 3, 2, or 1 amino acid variation) as compared with the HC CDR3 having the amino acid sequence of SEQ ID NO: 26. Alternatively or in addition, the anti-TMPRSS6 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 27; a LC CDR2 having no more than 3 amino acid variations (e.g., no more than 3, 2, or 1 amino acid variation) as compared with the LC CDR2 having the amino acid sequence of WAT; and/or a LC CDR3 having no more than 3 amino acid variations (e.g., no more than 3, 2, or 1 amino acid variation) as compared with the LC CDR3 having the amino acid sequence of SEQ ID NO: 29.

In some embodiments, an anti-TMPRSS6 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 24; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 48; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 26. Alternatively or in addition, the anti-TMPRSS6 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 27; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of WAT; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 29.

In some embodiments, an anti-TMPRSS6 antibody of the present disclosure comprises a VH comprising the amino acid sequence of SEQ ID NO: 49. Alternatively, or in addition, the anti-TMPRSS6 antibody of the present disclosure comprises a VL comprising the amino acid sequence of SEQ ID NO: 50.

In some embodiments, an anti-TMPRSS6 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 49. Alternatively, or in addition, the anti-TMPRSS6 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 50. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 49 and/or a VL of SEQ ID NO: 50 excluding any of the CDR sequences therein. In some embodiments, an anti-TMPRSS6 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 49, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 50.

In some embodiments, an anti-TMPRSS6 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) %) identical to the VH as set forth in SEQ ID NO: 49. Alternatively, or in addition, the anti-TMPRSS6 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 50. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 49, and/or a VL of SEQ ID NO: 50 excluding any of the CDR sequences therein. In some embodiments, an anti-TMPRSS6 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 49, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 50.

In some embodiments, an anti-TMPRSS6 antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, an anti-TMPRSS6 antibody of the present disclosure comprises a VH domain and/or VL domain of any one of the anti-TMPRSS6 antibodies selected from Table 1, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra.

In some embodiments, the light chain of any of the anti-TMPRSS6 antibodies described herein may further comprise a light chain constant region (CL), which can be any CL known in the art. In some examples, the CL is a kappa light chain. In other examples, the CL is a lambda light chain. In some embodiments, the CL is a kappa light chain.

Other antibody heavy and light chain constant regions are well known in the art, e.g., those provided in the IMGT database (www.imgt.org) or at www.vbase2.org/vbstat.php., both of which are incorporated by reference herein.

In some embodiments, an anti-TMPRSS6 antibody described herein comprises a heavy chain comprising any one of the VH as listed in Table 1 or any variants thereof, and a heavy chain constant region that is at least 80%, at least 85%, at least 90%, at least 91%, at least 92%, at least 93%, at least 94%, at least 95%, at least 96%, at least 97%, at least 98%, or at least 99% to SEQ ID NO: 9. In some embodiments, an anti-TMPRSS6 antibody described herein comprises a heavy chain comprising any one of the VH as listed in Table 1 or any variants thereof and a heavy chain constant region that contains no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with SEQ ID NO: 9. In some embodiments, an anti-TMPRSS6 antibody described herein comprises a heavy chain comprising any one of the VH as listed in Table 1 or any variants thereof and a heavy chain constant region set forth in SEQ ID NO: 9.

In some embodiments, an anti-TMPRSS6 antibody described herein comprises a heavy chain comprising any one of the VH as listed in Table 1 or any variants thereof and a heavy chain constant region that is at least 80%, at least 85%, at least 90%, at least 91%, at least 92%, at least 93%, at least 94%, at least 95%, at least 96%, at least 97%, at least 98%, or at least 99% identical to SEQ ID NO: 21. In some embodiments, an anti-TMPRSS6 antibody described herein comprises a heavy chain comprising any one of the VH as listed in Table 1 or any variants thereof and a heavy chain constant region that contains no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with SEQ ID NO: 21. In some embodiments, an anti-TMPRSS6 antibody described herein comprises a heavy chain comprising any one of the VH as listed in Table 1 or any variants thereof and a heavy chain constant region set forth in SEQ ID NO: 21.

In some embodiments, an anti-TMPRSS6 antibody described herein comprises a heavy chain comprising any one of the VH as listed in Table 1 or any variants thereof, and a heavy chain constant region that is at least 80%, at least 85%, at least 90%, at least 91%, at least 92%, at least 93%, at least 94%, at least 95%, at least 96%, at least 97%, at least 98%, or at least 99% identical to SEQ ID NO: 39. In some embodiments, an anti-TMPRSS6 antibody described herein comprises a heavy chain comprising any one of the VH as listed in Table 1 or any variants thereof and a heavy chain constant region that contains no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with SEQ ID NO: 39. In some embodiments, an anti-TMPRSS6 antibody described herein comprises a heavy chain comprising any one of the VH as listed in Table 1 or any variants thereof and a heavy chain constant region set forth in SEQ ID NO: 39.

In some embodiments, an anti-TMPRSS6 antibody described herein comprises a heavy chain comprising any one of the VH as listed in Table 1 or any variants thereof, and a heavy chain constant region that is at least 80%, at least 85%, at least 90%, at least 91%, at least 92%, at least 93%, at least 94%, at least 95%, at least 96%, at least 97%, at least 98%, or at least 99% identical to SEQ ID NO: 51. In some embodiments, an anti-TMPRSS6 antibody described herein comprises a heavy chain comprising any one of the VH as listed in Table 1 or any variants thereof and a heavy chain constant region that contains no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with SEQ ID NO: 51. In some embodiments, an anti-TMPRSS6 antibody described herein comprises a heavy chain comprising any one of the VH as listed in Table 1 or any variants thereof and a heavy chain constant region set forth in SEQ ID NO: 51.

In some embodiments, an anti-TMPRSS6 antibody described herein comprises a light chain comprising any one of the VL as listed in Table 1 or any variants thereof, and a light chain constant region that is at least 80%, at least 85%, at least 90%, at least 91%, at least 92%, at least 93%, at least 94%, at least 95%, at least 96%, at least 97%, at least 98%, or at least 99% to SEQ ID NO: 10. In some embodiments, an anti-TMPRSS6 antibody described herein comprises a light chain comprising any one of the VL as listed in Table 1 or any variants thereof and a light chain constant region that contains no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with SEQ ID NO: 10. In some embodiments, an anti-TMPRSS6 antibody described herein comprises a light chain comprising any one of the VL as listed in Table 1 or any variants thereof and a light chain constant region set forth in SEQ ID NO: 10.

In some embodiments, an anti-TMPRSS6 antibody described herein comprises a light chain comprising any one of the VL as listed in Table 1 or any variants thereof, and a light chain constant region that is at least 80%, at least 85%, at least 90%, at least 91%, at least 92%, at least 93%, at least 94%, at least 95%, at least 96%, at least 97%, at least 98%, or at least 99% to SEQ ID NO: 40. In some embodiments, an anti-TMPRSS6 antibody described herein comprises a light chain comprising any one of the VL as listed in Table 1 or any variants thereof and a light chain constant region that contains no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with SEQ ID NO: 40. In some embodiments, an anti-TMPRSS6 antibody described herein comprises a light chain comprising any one of the VL as listed in Table 1 or any variants thereof and a light chain constant region set forth in SEQ ID NO: 40. Examples of IgG heavy chain and light chain amino acid sequences of the anti-TMPRSS6 antibodies described are provided in Table 1 above.

In some embodiments, an anti-TMPRSS6 antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 11. Alternatively, or in addition, the anti-TMPRSS6 antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in SEQ ID NO: 12. In some embodiments, an anti-TMPRSS6 antibody described herein comprises a heavy chain comprising an amino acid sequence that is at least 80% at least 80%, at least 85%, at least 90%, at least 91%, at least 92%, at least 93%, at least 94%, at least 95%, at least 96%, at least 97%, at least 98%, or at least 99% to SEQ ID NO: 11. Alternatively or in addition, the anti-TMPRSS6 antibody described herein comprises a light chain comprising an amino acid sequence that is at least 80% at least 80%, at least 85%, at least 90%, at least 91%, at least 92%, at least 93%, at least 94%, at least 95%, at least 96%, at least 97%, at least 98%, or at least 99% identical to SEQ ID NO: 12. In some embodiments, an anti-TMPRSS6 antibody described herein comprises a heavy chain comprising the amino acid sequence of SEQ ID NO: 11. Alternatively, or in addition, the anti-TMPRSS6 antibody described herein comprises a light chain comprising the amino acid sequence of SEQ ID NO: 12.

In some embodiments, an anti-TMPRSS6 antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NOs: 17 or 22. Alternatively, or in addition, the anti-TMPRSS6 antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in SEQ ID NO: 23. In some embodiments, an anti-TMPRSS6 antibody described herein comprises a heavy chain comprising an amino acid sequence that is at least 80% (e.g., 80%, 85%, 90%, 91%, 92%, 93%, 94%, 95%, 96%, 97%, 98%, or 99%) identical to SEQ ID NOs: 17 or 22. Alternatively, or in addition, the anti-TMPRSS6 antibody described herein comprises a light chain comprising an amino acid sequence that is at least 80% (e.g., 80%, 85%, 90%, 91%, 92%, 93%, 94%, 95%, 96%, 97%, 98%, or 99%) identical to SEQ ID NO: 23. In some embodiments, an anti-TMPRSS6 antibody described herein comprises a heavy chain comprising the amino acid sequence of SEQ ID NOs: 17 or 22. Alternatively, or in addition, the anti-TMPRSS6 antibody described herein comprises a light chain comprising the amino acid sequence of SEQ ID NO: 23.

In some embodiments, an anti-TMPRSS6 antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 33. Alternatively, or in addition, the anti-TMPRSS6 antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in SEQ ID NO: 34. In some embodiments, an anti-TMPRSS6 antibody described herein comprises a heavy chain comprising an amino acid sequence that is at least 80% (e.g., 80%, 85%, 90%, 91%, 92%, 93%, 94%, 95%, 96%, 97%, 98%, or 99%) identical to SEQ ID NO: 33. Alternatively, or in addition, the anti-TMPRSS6 antibody described herein comprises a light chain comprising an amino acid sequence that is at least 80% (e.g., 80%, 85%, 90%, 91%, 92%, 93%, 94%, 95%, 96%, 97%, 98%, or 99%) identical to SEQ ID NO: 34. In some embodiments, an anti-TMPRSS6 antibody described herein comprises a heavy chain comprising the amino acid sequence of SEQ ID NO: 33. Alternatively, or in addition, the anti-TMPRSS6 antibody described herein comprises a light chain comprising the amino acid sequence of SEQ ID NO: 34.

In some embodiments, an anti-TMPRSS6 antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 41. Alternatively or in addition, the anti-TMPRSS6 antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in SEQ ID NO: 42. In some embodiments, an anti-TMPRSS6 antibody described herein comprises a heavy chain comprising an amino acid sequence that is at least 80% (e.g., 80%, 85%, 90%, 91%, 92%, 93%, 94%, 95%, 96%, 97%, 98%, or 99%) identical to SEQ ID NO: 41. Alternatively, or in addition, the anti-TMPRSS6 antibody described herein comprises a light chain comprising an amino acid sequence that is at least 80% (e.g., 80%, 85%, 90%, 91%, 92%, 93%, 94%, 95%, 96%, 97%, 98%, or 99%) identical to SEQ ID NO: 42. In some embodiments, an anti-TMPRSS6 antibody described herein comprises a heavy chain comprising the amino acid sequence of SEQ ID NO: 41. Alternatively, or in addition, the anti-TMPRSS6 antibody described herein comprises a light chain comprising the amino acid sequence of SEQ ID NO: 42.

In some embodiments, an anti-TMPRSS6 antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 46. Alternatively, or in addition, the anti-TMPRSS6 antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in SEQ ID NO: 47. In some embodiments, an anti-TMPRSS6 antibody described herein comprises a heavy chain comprising an amino acid sequence that is at least 80% (e.g., 80%, 85%, 90%, 91%, 92%, 93%, 94%, 95%, 96%, 97%, 98%, or 99%) identical to SEQ ID NO: 46. Alternatively, or in addition, an anti-TMPRSS6 antibody described herein comprises a light chain comprising an amino acid sequence that is at least 80% (e.g., 80%, 85%, 90%, 91%, 92%, 93%, 94%, 95%, 96%, 97%, 98%, or 99%) identical to SEQ ID NO: 47. In some embodiments, an anti-TMPRSS6 antibody described herein comprises a heavy chain comprising the amino acid sequence of SEQ ID NO: 46. Alternatively, or in addition, the anti-TMPRSS6 antibody described herein comprises a light chain comprising the amino acid sequence of SEQ ID NO: 47.

In some embodiments, an anti-TMPRSS6 antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 52. Alternatively or in addition, the anti-TMPRSS6 antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in SEQ ID NO: 53. In some embodiments, an anti-TMPRSS6 antibody described herein comprises a heavy chain comprising an amino acid sequence that is at least 80% (e.g., 80%, 85%, 90%, 91%, 92%, 93%, 94%, 95%, 96%, 97%, 98%, or 99%) identical to SEQ ID NO: 52. Alternatively, or in addition, the anti-TMPRSS6 antibody described herein comprises a light chain comprising an amino acid sequence that is at least 80% (e.g., 80%, 85%, 90%, 91%, 92%, 93%, 94%, 95%, 96%, 97%, 98%, or 99%) identical to SEQ ID NO: 53. In some embodiments, an anti-TMPRSS6 antibody described herein comprises a heavy chain comprising the amino acid sequence of SEQ ID NO: 52. Alternatively, or in addition, the anti-TMPRSS6 antibody described herein comprises a light chain comprising the amino acid sequence of SEQ ID NO: 53.

The anti-TMPRSS6 antibodies described herein can be in any antibody form, including, but not limited to, intact (i.e., full-length) antibodies, antigen-binding fragments thereof (such as Fab, F(ab′), F(ab′)2, Fv), single chain antibodies, bi-specific antibodies, or nanobodies. In some embodiments, an anti-TMPRSS6 antibody described herein is a scFv. In some embodiments, an anti-TMPRSS6 antibody described herein is a scFv-Fab (e.g., scFv fused to a portion of a constant region).

In some embodiments, conservative mutations can be introduced into antibody sequences (e.g., CDRs or framework sequences) at positions where the residues are not likely to be involved in interacting with a target antigen (e.g., TMPRSS6), for example, as determined based on a crystal structure. In some embodiments, one, two or more mutations (e.g., amino acid substitutions) are introduced into the Fc region of an anti-TMPRSS6 antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of an antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109:6181-6186, U.S. Pat. No. 6,737,056, and International Publication No.; WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions, or deletions) are introduced into an IgG constant domain, or FcRn-binding fragment thereof (preferably an Fc or hinge-Fc domain fragment) to decrease the half-life of the anti-TMPRSS6 antibody in vivo. In some embodiments, one, two or more amino acid mutations (i.e., substitutions, insertions, or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine(S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281:23514-24).

In some embodiments, one, two or more amino acid substitutions are introduced into an IgG constant domain Fc region to alter the effector function(s) of the anti-TMPRSS6 antibody. The effector ligand to which affinity is altered can be, for example, an Fc receptor or the C1 component of complement. This approach is described in further detail in U.S. Pat. Nos. 5,624,821 and 5,648,260. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276:6591-604).

In some embodiments, one or more amino in the constant region of an anti-TMPRSS6 antibody described herein can be replaced with a different amino acid residue such that the antibody has altered Clq binding and/or reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or to increase the affinity of the antibody for an Fcγ receptor. This approach is described further in International Publication No. WO 00/42072.

In some embodiments, the heavy and/or light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind TMPRSS6, such that the variant, CDR-grafted, chimeric, humanized, or composite antibody has at least 50%, at least 60%, at least 70%, at least 80%, at least 90%, at least 95% or more binding to TMPRSS6 relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., A single amino acid substitution abolishes the heterogeneity of chimeric mouse/human (IgG4) antibody, Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, one or more sugar or carbohydrate molecules are conjugated to the antibody via N-glycosylation, O-glycosylation, C-glycosylation, glypiation (GPI anchor attachment), and/or phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, any one of the anti-TMPRSS6 antibodies described herein may comprise a signal peptide in the heavy and/or light chain sequence (e.g., a N-terminal signal peptide). In some embodiments, the anti-TMPRSS6 antibody described herein comprises any one of the VH and VL sequences, any one of the IgG heavy chain and light chain sequences, or any one of the F(ab′) heavy chain and light chain sequences described herein, and further comprises a signal peptide (e.g., a N-terminal signal peptide).

### III. Preparation of the Anti-TMPRSS6 Antibodies

Antibodies capable of binding TMPRSS6 as described herein can be made by any method known in the art. See, for example, Harlow and Lane, (1998) Antibodies: A Laboratory Manual, Cold Spring Harbor Laboratory, New York.

In some embodiments, antibodies specific to a target antigen (e.g., TMPRSS6) can be made by the conventional hybridoma technology. The full-length target antigen or a fragment thereof, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, CA) and HuMAb-Mouse® and TC Mouse™ from Medarex, Inc. (Princeton, NJ) or H2L2 mice from Harbour Antibodies BV (Holland). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments. Genetically engineered antibodies, such as humanized antibodies, chimeric antibodies, single-chain antibodies, and bi-specific antibodies, can be produced via, e.g., conventional recombinant technology. In one example, DNA encoding a monoclonal antibody specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, human HEK293 cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

Alternatively, techniques described for the production of single chain antibodies (U.S. Pat. Nos. 4,946,778 and 4,704,692) can be adapted to produce a phage or yeast scFv library and scFv clones specific to TMPRSS6 can be identified from the library following routine procedures. Positive clones can be subjected to further screening to identify those that has high TMPRSS6 binding affinity.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In one example, epitope mapping can be accomplished use H/D-Ex (hydrogen deuterium exchange) coupled with proteolysis and mass spectrometry. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. Alternatively, competition assays can be performed using other antibodies known to bind to the same antigen to determine whether an antibody binds to the same epitope as the other antibodies. Competition assays are well known to those of skill in the art.

In some examples, an anti-TMPRSS6 antibody is prepared by recombinant technology as exemplified below. Nucleic acids encoding the heavy and light chain of an anti-TMPRSS6 antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV promoter, and the herpes simplex tk virus promoter.

Regulatable promoters can also be used. Such regulatable promoters include those using the lac repressor from E. coli as a transcription modulator to regulate transcription from lac operator bearing mammalian cell promoters (Brown, M. et al., Cell, 49:603-612 (1987)), those using the tetracycline repressor (tetR) (Gossen, M., and Bujard, H., Proc. Natl. Acad. Sci. USA 89:5547-555115 (1992); Yao, F. et al., Human Gene Therapy, 9:1939-1950 (1998); Shockelt, P., et al., Proc. Natl. Acad. Sci. USA, 92:6522-6526 (1995)). Other systems include FK506 dimer, VP16 or p65 using astradiol, RU486, diphenol murislerone, or rapamycin. Inducible systems are available from Invitrogen, Clontech and Ariad, among others.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987)); Gossen and Bujard (1992); (M. Gossen et al., Natl. Acad. Sci. USA, 89:5547-5551 (1992)) combined the tetracycline repressor (tetR) with the transcription activator (VP 16) to create a tetR-mammalian cell transcription activator fusion protein, tTa (tetR-VP 16), with the tetO bearing minimal promoter derived from the human cytomegalovirus (hCMV) promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy). One particular advantage of this tetracycline inducible switch is that it does not require the use of a tetracycline repressor-mammalian cells transactivator or repressor fusion protein, which in some instances can be toxic to cells (Gossen 5 et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColEl for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art. Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies (e.g., the nucleic acid coding sequence listed in Table 3) may be introduced into suitable host cells for producing the antibodies. Non-limiting examples of the host cells include Chinese hamster ovary (CHO) cells, dhfr-CHO cell, human embryonic kidney (HEK)-293 cells, verda reno (VERO) cells, nonsecreting null (NSO) cells, human embryonic retinal (PER.C6) cells, Sp2/0 cells, baby hamster kidney (BHK) cells, Madin-Darby Canine Kidney (MDCK) cells, Madin-Darby Bovine Kidney (MDBK) cells, and monkey kidney CV1 line transformed by SV40 (COS) cells. In some embodiments, the host cell expressing the anti-TMPRSS6 antibodies are CHO cells. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody. In some embodiments, the host cell comprises the nucleic acid encoding the heavy chain of the anti-TMPRSS6 antibody. In some embodiments, the host cell comprises the nucleic acid encoding the light chain of the anti-TMPRSS6 antibody. In some embodiments, the host cell comprises the nucleic acid encoding the heavy chain and the nucleic acid encoding the light chain.

In some embodiments, methods for preparing an antibody described herein involve a recombinant expression vector that encodes both the heavy chain and the light chain of an anti-TMPRSS6 antibody, as also described herein. The recombinant expression vector can be introduced into a suitable host cell (e.g., a dhfr-CHO cell) by a conventional method, e.g., calcium phosphate mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-TMPRSS6 antibody and the other encoding the light chain of the anti-TMPRSS6 antibody. Both of the two recombinant expression vectors can be introduced into a suitable host cell (e.g., dhfr-CHO cell) by a conventional method, e.g., calcium phosphate-mediated transfection.

Alternatively, each of the expression vectors can be introduced into a suitable host cell. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-TMPRSS6 antibody as described herein (e.g., as provided in Table 3), vectors (e.g., expression vectors) containing such; and host cells comprising the vectors are within the scope of the present disclosure.

TABLE 3
Nucleic acids Sequences encoding the VH/VL of anti-TMPRSS6
antibodies listed in Table 1

| Antibody | | Nucleic Acid Sequence | SEQ ID NO |
|---|---|---|---|
| A1 | VH | caggtccaactgcagcagcctggggctgagctt | 54 |
| | | gcgaagcctggggcttcagtgaagatgtcctgc | |
| | | aaggcttctggctacaccttcaccagctactgg | |
| | | ataacctgggtgaagcagaggcctggacaagac | |
| | | cttgagtggattggaaatatttatcctggtagt | |
| | | ggtagtacttactacaatgagaagttcaagagc | |
| | | aaggccacactgactgtagacacatcctccaga | |
| | | acagcctacatgcagctcagcagtctgacatct | |
| | | gcggactctgcggtctattactgtgccccctat | |
| | | gattccgactatgctatggactactggggtcaa | |
| | | ggaacctcagtcaccgtctcctca | |
| | VL | gacatcaagatgacccagtctccatcttccatg | 55 |
| | | tatgcatctctaggagagagagtcactatcact | |
| | | tgcaaggcgagtcaggacattaataactattta | |
| | | agctggttccagcagaaaccagggaaatctcct | |
| | | aagaccctgatctatcgtgcaaacagattggta | |
| | | gatggggtcccatcaagggtcagtggcagtgga | |
| | | tctgggcaagattattctctcaccatcagcagc | |
| | | ctggagtatgaagatgtgggaatttatttttgt | |
| | | ctacagtatgatgagtttcctctcacgttcggt | |
| | | gctgggaccaagctggagctgaaa | |
| | Heavy | caggtccaactgcagcagcctggggctgagctt | 56 |
| | Chain | gcgaagcctggggcttcagtgaagatgtcctgc | |
| | | aaggcttctggctacaccttcaccagctactgg | |
| | | ataacctgggtgaagcagaggcctggacaagac | |
| | | cttgagtggattggaaatatttatcctggtagt | |
| | | ggtagtacttactacaatgagaagttcaagagc | |
| | | aaggccacactgactgtagacacatcctccaga | |
| | | acagcctacatgcagctcagcagtctgacatct | |
| | | gcggactctgcggtctattactgtgccccctat | |
| | | gattccgactatgctatggactactggggtcaa | |
| | | ggaacctcagtcaccgtctcctcagctaaaaca | |
| | | acagccccatcggtctatccactggcccctgtg | |
| | | tgtggagatacaactggctcctcggtgactcta | |
| | | ggatgcctggtcaagggttatttccctgagcca | |
| | | gtgaccttgacctggaactctggttccctgtcc | |
| | | agtggtgtgcacaccttcccagctgtcctgcag | |
| | | tctgacctctacaccctcagctcaagcgtgact | |
| | | gtaaccagctcgacctggcccagccagtccatc | |
| | | acctgcaatgtggcccacccggcaagcagcacc | |
| | | aaggtggacaagaaaattgagcccagagggccc | |
| | | acaatcaagccctgtcctccatgcaaatgccca | |
| | | gcacctaacctcttgggtggaccatccgtcttc | |
| | | atcttccctccaaagatcaaggatgtactcatg | |
| | | atctccctgagccccatagtcacatgtgtagtc | |
| | | gttgatgtgagcgaggatgacccagatgtccag | |
| | | atcagctggtttgtgaacaacgtggaagtgcac | |
| | | actgctcagacacagacgcatagagaggattac | |
| | | aacagtactctccgggttgtcagtgccctcccc | |
| | | atccagcaccaggactggatgagtggcaaggag | |
| | | ttcaaatgcaaggtcaacaacaaagacctccca | |
| | | gcgcccatcgagagaaccatctcaaaacccaaa | |
| | | gggtcagtaagagctccacaggtatatgtcttg | |
| | | cctccaccagaagaggagatgactaagaaacag | |
| | | gtcactctgacctgcatggtcacagacttcatg | |
| | | cctgaagacatttacgtggagtggaccaacaac | |
| | | gggaaaacagagctaaactacaagaacactgaa | |
| | | ccagtcctggactctgatggttcttacttcatg | |
| | | tacagcaagctgagagtggagaagaagaactgg | |
| | | gtggagagaaatagctactcctgttcagtggtc | |
| | | cacgagggtctgcacaatcaccacacgactaag | |
| | | agcttctcccggactccgggttagtaa | |
| | Light | gacatcaagatgacccagtctccatcttccatg | 57 |
| | Chain | tatgcatctctaggagagagagtcactatcact | |
| | | tgcaaggcgagtcaggacattaataactattta | |
| | | agctggttccagcagaaaccagggaaatctcct | |
| | | aagaccctgatctatcgtgcaaacagattggta | |
| | | gatggggtcccatcaaggg | |
| | | tcagtggcagtggatctgggcaagattattctc | |
| | | tcaccatcagcagcctggagtatgaagatgtgg | |
| | | gaatttatttttgtctacagtatgatgagtttc | |
| | | ctctcacgttcggtgctgggaccaagctggagc | |
| | | tgaaaagagctgacgccgctcctaccgtgtcca | |
| | | tctttccacctagcagcgagcagctgacaagcg | |
| | | gcggagccagcgtcgtgtgcttcctgaacaact | |
| | | tctaccccaaggacatcaacgtgaagtggaaga | |
| | | tcgacggcagcgagagacagaacggcgtgctga | |
| | | atagctggaccgaccaggacagcaaggactcca | |
| | | cctacagcatgtccagcacactgaccctgacca | |
| | | aggacgagtacgagcggcacaacagctacacat | |
| | | gcgaggccacacacaagaccagcacaagcccca | |
| | | tcgtgaagtccttcaaccggaacgagtgc | |
| B1 | VH | gaggttcagctgcagcagtctggggcagaactt | 58 |
| | | gtgaagccaggggcctcagtcaagttgtcctgc | |
| | | acagcctctggcttcaacattaaagactactat | |
| | | atacactgggtgaaagagaggactgaacagggc | |
| | | ctggagtggtttggaaggattgatcctgaggat | |
| | | ggtgaaagtgaatatgccccgaaattccagggc | |
| | | aaggccactttaacagcagacacatcctccaat | |
| | | acagcctacctgcagctcagcagcctgacatct | |
| | | gaggacactgccgtctattactgtactagagga | |
| | | gactctatgatggttacctactttgactactgg | |
| | | ggccaaggcaccactctcacggtctcctca | |
| | VL | gacattgtgatgacccagtctcacaaattcatg | 59 |
| | | tccacatcagtaggagacagggtcagcatcacc | |
| | | tgcaaggccagtcaggatgtgagtactgctgta | |
| | | gcctggtatcaacaaaaaccagggcaatctcct | |
| | | aaactactgatttactgggctttcacccgtcac | |
| | | actggagtccctgatcgcttcacaagcactgga | |
| | | tctgggacagattatgctctcaccatcagcagt | |
| | | gtgcaggctgaagacctggcactttattactgt | |
| | | cagcaacattatcgcagtccgtggacgttcggt | |
| | | ggaggcaccaaactggaaatcaaa | |
| | Heavy | gaggttcagctgcagcagtctggggcagaactt | 60 |
| | Chain | gtgaagccaggggcctcagtcaagttgtcctgc | |
| | | acagcctctggcttcaacattaaagactactat | |
| | | atacactgggtgaaagagaggactgaacagggc | |
| | | ctggagtggtttggaaggattgatcctgaggat | |
| | | ggtgaaagtgaatatgccccgaaattccagggc | |
| | | aaggccactttaacagcagacacatcctccaat | |
| | | acagcctacctgcagctcagcagcctgacatct | |
| | | gaggacactgccgtctattactgtactagagga | |
| | | gactctatgatggttacctactttgactactgg | |
| | | ggccaaggcaccactctcacggtctcctcaaag | |
| | | accacacctcctagcgtgtaccctctggctcct | |
| | | ggctgtggcgatacaacaggcagctctgtgaca | |
| | | ctgggctgcctggtcaagggctactttcctgag | |
| | | agcgtgacagtgacctggaacagcggcagcctg | |
| | | tctagcagcgtgcacacctttccagctctgctc | |
| | | cagagcggcctgtacaccatgtcctctagtgtg | |
| | | accgtgcctagcagcacctggcctagccagaca | |
| | | gtgacatgtagcgtggcccatcctgccagcagc | |
| | | acaaccgtggacaagaagctggaacctagcggc | |
| | | cccatcagcaccatcaatccctgtcctccatgc | |
| | | aaagaatgccacaagtgccccgctcctaacctg | |
| | | gaaggtggcccaagcgtgttcatcttcccacct | |
| | | aacatcaaggacgtgctgatgatcagcctgaca | |
| | | cctaaagtgacctgcgtggtggtggacgtgtcc | |
| | | gaggatgatcccgatgtgcagatcagttggttc | |
| | | gtgaacaacgtggaagtgcacacagcccagaca | |
| | | cagacccacagagaggactacaatagcaccatt | |
| | | cgcgtggtgtccacactgcctatccagcaccag | |
| | | gattggatgagcggcaaagagttcaagtgcaaa | |
| | | gtgaacaacaaggacctgccttctccaatcgag | |
| | | cggaccatcagcaagatcaagggactcgtcaga | |
| | | gcccctcaggtgtacatcttgcctccaccagcc | |
| | | gagcagctgagcagaaaggatgtgtccctgacc | |
| | | tgtctggtcgtgggcttcaaccctggcgacatc | |
| | | agcgtggaatggaccagcaatggccacaccgag | |
| | | gaaaactacaaggacacagcccctgtgctggac | |
| | | agcgacggcagctacttcatctacagcaagctg | |
| | | aacatgaagaccagcaagtgggagaaaaccgac | |
| | | agcttctcctgcaacgtgcggcacgagggcctg | |
| | | aagaactactacctgaagaaaaccatctctcgg | |
| | | agccccggcaag | |
| | Light | gacattgtgatgacccagtctcacaaattcatg | 61 |
| | Chain | tccacatcagtaggagacagggtcagcatcacc | |
| | | tgcaaggccagtcaggatgtgagtactgctgta | |
| | | gcctggtatcaacaaaaaccagggcaatctcct | |
| | | aaacta | |
| | | ctgatttactgggctttcacccgtcacactgga | |
| | | gtccctgatcgcttcacaagcactggatctggg | |
| | | acagattatgctctcaccatcagcagtgtgcag | |
| | | gctgaagacctggcactttattactgtcagcaa | |
| | | cattatcgcagtccgtggacgttcggtggaggc | |
| | | accaaactggaaatcaaaagagctgacgccgct | |
| | | cctaccgtgtccatctttccacctagcagcgag | |
| | | cagctgacaagcggcggagccagcgtcgtgtgc | |
| | | ttcctgaacaacttctaccccaaggacatcaac | |
| | | gtgaagtggaagatcgacggcagcgagagacag | |
| | | aacggcgtgctgaatagctggaccgaccaggac | |
| | | agcaaggactccacctacagcatgtccagcaca | |
| | | ctgaccctgaccaaggacgagtacgagcggcac | |
| | | aacagctacacatgcgaggccacacacaagacc | |
| | | agcacaagccccatcgtgaagtccttcaaccgg | |
| | | aacgagtgc | |
| C1 | VH | gaagttcagctgcagcagtctggggcagaactt | 62 |
| | | gtgaagccaggggcctcagtcaagttgtcctgc | |
| | | acagcttctggcttcaacattgaagactactat | |
| | | atacactgggtgaaggagaggactgaacagggc | |
| | | ctggagtggattggaaggattgatcctgaggat | |
| | | ggtgaaactacatatgccccgcagttccagggc | |
| | | aaggccactataataccagacacatcctccaac | |
| | | acagcctacatgcagctcagcagcctgacatct | |
| | | gaggacgctgccgtctattactgtgctagatcg | |
| | | atctaccttgatcctatggactactggggtcaa | |
| | | ggaacctcagtcaccgtctcctca | |
| | VL | gacattgtgatgacccagtctcacaaattcatg | 63 |
| | | tccacatcagtaggagacagggtcagcatcacc | |
| | | tgcaaggccagtcaggatgtgactactgctgtc | |
| | | gcctggtatcaacaaaaaccaggacagtctcct | |
| | | aaaatactgatttactgggcaaccacccggcac | |
| | | actggagtccctgatcgcttcacaggcagtata | |
| | | tctgggacaacttatattctcaccatcagtagt | |
| | | gtgcaggctgaagacctggcactttattactgt | |
| | | cagcaacattatagcactccgtacacgttcgga | |
| | | ggggggaccaagctggaaaaaaa | |
| | Heavy | gaagttcagctgcagcagtctggggcagaactt | 5 |
| | Chain | gtgaagccaggggcctcagtcaagttgtcctgc | |
| | | acagcttctggcttcaacattgaagactactat | |
| | | atacactgggtgaaggagaggactgaacagggc | |
| | | ctggagtggattggaaggattgatcctgaggat | |
| | | ggtgaaactacatatgccccgcagttccagggc | |
| | | aaggccactataataccagacacatcctccaac | |
| | | acagcctacatgcagctcagcagcctgacatct | |
| | | gaggacgctgccgtctattactgtgctagatcg | |
| | | atctaccttgatcctatggactactggggtcaa | |
| | | ggaacctcagtcaccgtctcctcaaagaccaca | |
| | | cctcctagcgtgtaccctctggctcctggctgt | |
| | | ggcgatacaacaggcagctctgtgacactgggc | |
| | | tgcctggtcaagggctactttcctgagagcgtg | |
| | | acagtgacctggaacagcggcagcctgtctagc | |
| | | agcgtgcacacctttccagctctgctccagagc | |
| | | ggcctgtacaccatgtcctctagtgtgaccgtg | |
| | | cctagcagcacctggcctagccagacagtgaca | |
| | | tgtagcgtggcccatcctgccagcagcacaacc | |
| | | gtggacaagaagctggaacctagcggccccatc | |
| | | agcaccatcaatccctgtcctccatgcaaagaa | |
| | | tgccacaagtgccccgctcctaacctggaaggt | |
| | | ggcccaagcgtgttcatcttcccacctaacatc | |
| | | aaggacgtgctgatgatcagcctgacacctaaa | |
| | | gtgacctgcgtggtggtggacgtgtccgaggat | |
| | | gatcccgatgtgcagatcagttggttcgtgaac | |
| | | aacgtggaagtgcacacagcccagacacagacc | |
| | | cacagagaggactacaatagcaccattcgcgtg | |
| | | gtgtccacactgcctatccagcaccaggattgg | |
| | | atgagcggcaaagagttcaagtgcaaagtgaac | |
| | | aacaaggacctgccttctccaatcgagcggacc | |
| | | atcagcaagatcaagggactcgtcagagcccct | |
| | | caggtgtacatcttgcctccaccagccgagcag | |
| | | ctgagcagaaaggatgtgtccctgacctgtctg | |
| | | gtcgtgggcttcaaccctggcgacatcagcgtg | |
| | | gaatggaccagcaatggccacaccgaggaaaac | |
| | | tacaaggacacagcccctgtgctggacagcgac | |
| | | ggcagctacttcatctacagcaagctgaacatg | |
| | | aagaccagcaagtgggagaaaaccgacagcttc | |
| | | tcctgcaacgtgcggcacgagggcctgaagaac | |
| | | tactacctgaagaaaaccatctctcggagcccc | |
| | | ggcaag | |
| | Light | Gacattgtgatgacccagtctcacaaattcatg | 32 |
| | Chain | tccacatcagtaggagacagggtcagcatcacc | |
| | | tgcaaggccagtcaggatgtgactactgctgtc | |
| | | gcctggtatcaacaaaaaccaggacagtctcct | |
| | | aaaatactgatttactgggcaaccacccggcac | |
| | | actggagtccctgatcgcttcacaggcagtata | |
| | | tctgggacaacttatattctcaccatcagtagt | |
| | | gtgcaggctgaagacctggcactttattactgt | |
| | | cagcaacattatagcactccgtacacgttcgga | |
| | | ggggggaccaagctggaaataaaaagagctgac | |
| | | gccgctcctaccgtgtccatctttccacctagc | |
| | | agcgagcagctgacaagcggcggagccagcgtc | |
| | | gtgtgcttcctgaacaacttctaccccaaggac | |
| | | atcaacgtgaagtggaagatcgacggcagcgag | |
| | | agacagaacggcgtgctgaatagctggaccgac | |
| | | caggacagcaaggactccacctacagcatgtcc | |
| | | agcacactgaccctgaccaaggacgagtacgag | |
| | | cggcacaacagctacacatgcgaggccacacac | |
| | | aagaccagcacaagccccatcgtgaagtccttc | |
| | | aaccggaacgagtgc | |
| | VH | ctactacatccactgggtcaaagagcggaccga | 79 |
| | | gcagggactcgagtggatcggaagaatcgaccc | |
| | | cgaggacggcgagacaacatacgcccctcagtt | |
| | | tcagggcaaagccacaatcatccccgacaccag | |
| | | cagcaacaccgcctacatgcaactgagcagcct | |
| | | gacctctgaagatgccgccgtgtactactgcgc | |
| | | ccggtccatctatctggaccccatggattattg | |
| | | gggccagggcacaagcgtgaccgtgtcctct | |
| | VL | Gacatcgtgatgacccagagccacaagttcatg | 28 |
| | | agcaccagcgtgggcgacagagtgtccatcacc | |
| | | tgtaaagccagccaggacgtgacaacagccgtg | |
| | | gcctggtatcagcagaagcctggccagtctcct | |
| | | aagatcctgatctactgggccaccaccagacac | |
| | | accggcgtgccagatagattcaccggcagcatc | |
| | | agcggcaccacctacatcctgacaatcagctct | |
| | | gtgcaggccgaggatctggccctgtactactgt | |
| | | cagcagcactacagcaccccttacacctttggc | |
| | | ggaggcaccaagctggaaatcaag | |
| | Heavy | Gaggttcagctgcagcagtctggcgccgagctt | 64 |
| | Chain | gtgaaacctggcgcctctgtgaagctgagctgt | |
| | | accgccagcggcttcaacatcgaggactactac | |
| | | atccactgggtcaaagagcggaccgagcaggga | |
| | | ctcgagtggatcggaagaatcgaccccgaggac | |
| | | ggcgagacaacatacgcccctcagtttcagggc | |
| | | aaagccacaatcatccccgacaccagcagcaac | |
| | | accgcctacatgcaactgagcagcctgacctct | |
| | | gaagatgccgccgtgtactactgcgcccggtcc | |
| | | atctatctggaccccatggattattggggccag | |
| | | ggcacaagcgtgaccgtgtcctctaagaccaca | |
| | | cctcctagcgtgtaccctctggctcctggctgt | |
| | | ggcgatacaacaggcagctctgtgacactgggc | |
| | | tgcctggtcaagggctactttcctgagagcgtg | |
| | | acagtgacctggaacagcggcagcctgtctagc | |
| | | agcgtgcacacctttccagctctgctccagagc | |
| | | ggcctgtacaccatgtcctctagtgtgaccgtg | |
| | | cctagcagcacctggcctagccagacagtgaca | |
| | | tgtagcgtggcccatcctgccagcagcacaacc | |
| | | gtggacaagaagctggaacctagcggccccatc | |
| | | agcaccatcaatccctgtcctccatgcaaagaa | |
| | | tgccacaagtgccccgctcctaacctggaaggt | |
| | | ggcccaagcgtgttcatcttcccacctaacatc | |
| | | aaggacgtgctgatgatcagcctgacacctaaa | |
| | | gtgacctgcgtggtggtggacgtgtccgaggat | |
| | | gatcccgatgtgcagatcagttggttcgtgaac | |
| | | aacgtggaagtgcacacagcccagacacagacc | |
| | | cacagagaggactacaatagcaccattcgcgtg | |
| | | gtgtccacactgcctatccagcaccaggattgg | |
| | | atgagcggcaaagagttcaagtgcaaagtgaac | |
| | | aacaaggacctgccttctccaatcgagcggacc | |
| | | atcagcaagatcaagggactcgtcagagcccct | |
| | | caggtgtacatcttgcctccaccagccgagcag | |
| | | ctgagcagaaaggatgtgtccctgacctgtctg | |
| | | gtcgtgggcttcaaccctggcgacatcagcgtg | |
| | | gaatggaccagcaatggccacaccgaggaaaac | |
| | | tacaaggacacagcccctgtgctggacagcgac | |
| | | ggcagctacttcatctacagcaagctgaacatg | |
| | | aagaccagcaagtgggagaaaaccgacagcttc | |
| | | tcctgcaacgtgcggcacgagggcctgaagaac | |
| | | tactacctgaagaaaaccatctctcggagcccc | |
| | | ggcaag | |
| | Light | Gacatcgtgatgacccagagccacaagttcatg | 65 |
| | Chain | agcaccagcgtgggcgacagagtgtccatcacc | |
| | | tgtaaagccagccaggacgtgacaacagccgtg | |
| | | gcctggtatcagcagaagcctggccagtctcct | |
| | | aagatcctgatctactgggccaccaccagacac | |
| | | accggcgtgccagatagattcaccggcagcatc | |
| | | agcggcaccacctacatcctgacaatcagctct | |
| | | gtgcaggccgaggatctggccctgtactactgt | |
| | | cagcagcactacagcaccccttacacctttggc | |
| | | ggaggcaccaagctggaaatcaagagagctgac | |
| | | gccgctcctaccgtgtccatctttccacctagc | |
| | | agcgagcagctgacaagcggcggagccagcgtc | |
| | | gtgtgcttcctgaacaacttctaccccaaggac | |
| | | atcaacgtgaagtggaagatcgacggcagcgag | |
| | | agacagaacggcgtgctgaatagctggaccgac | |
| | | caggacagcaaggactccacctacagcatgtcc | |
| | | agcacactgaccctgaccaaggacgagtacgag | |
| | | cggcacaacagctacacatgcgaggccacacac | |
| | | aagaccagcacaagccccatcgtgaagtccttc | |
| | | aaccggaacgagtgc | |
| A2 | VH | gaagtgcagctggtgcaatctggcgccgaagtg | 66 |
| | | aagaaacctggcgcctctgtgaaggtgtcctgc | |
| | | aaggcttccggctacacctttaccagctactgg | |
| | | atcacctgggtccgacaggctcctggccagaga | |
| | | ctggaatggatcggcaacatctaccctggctcc | |
| | | ggctccacctactacaacgagaagttcaagtcc | |
| | | aaggccacaatcacccgggacacctcttccaga | |
| | | accgcctacatggaactgtccagcctgagatct | |
| | | gaggacaccgccgtgtactactgcgccccttac | |
| | | gacgccgactacgccatggattattggggccag | |
| | | ggcaccctggtcaccgtgtcctct | |
| | VL | gacatccagatgacccagtctccatcctctctg | 67 |
| | | tccgcctctgtgggcgacagagtgaccatcaca | |
| | | tgcaaggccagccaggacatctccaactacctg | |
| | | tcctggttccagcagaagcctggcaaggctccc | |
| | | aagctgctgatctacagagccaacagactggtg | |
| | | gaaggcgtgccctccagattctccggatctggc | |
| | | tctggcaccgactttaccctgacaatctccagc | |
| | | ctgcagcctgaggacttcgctacctacttctgc | |
| | | ctgcaatacgacgagttccctctgacctttggc | |
| | | ggaggcaccaaggtggaaatcaag | |
| | Heavy | gaagtgcagctggtgcaatctggcgccgaagtg | 68 |
| | Chain | aagaaacctggcgcctctgtgaaggtgtcctgc | |
| | | aaggcttccggctacacctttaccagctactgg | |
| | | atcacctgggtccgacaggctcctggccagaga | |
| | | ctggaatggatcggcaacatctaccctggctcc | |
| | | ggctccacctactacaacgagaagttcaagtcc | |
| | | aaggccacaatcacccgggacacctcttccaga | |
| | | accgcctacatggaactgtccagcctgagatct | |
| | | gaggacaccgccgtgtactactgcgccccttac | |
| | | gacgccgactacgccatggattattggggccag | |
| | | ggcaccctggtcaccgtgtcctctgcttctacc | |
| | | aagggacccagcgtgttccctctggctccttcc | |
| | | agcaagtctacctctggcggaacagctgctctg | |
| | | ggctgcctggtcaaggactactttcctgagcct | |
| | | gtgaccgtgtcttggaactctggcgctctgaca | |
| | | tccggcgtgcacacatttccagctgtgctgcag | |
| | | tcctccggcctgtactctctgtcctctgtcgtg | |
| | | accgtgccttcctctagcctgggcacccagacc | |
| | | tacatctgcaatgtgaaccacaagccttccaac | |
| | | accaaggtggacaagaaggtggaacccaagtcc | |
| | | tgcgacaagacccacacctgtcctccatgtcct | |
| | | gctccagaactgctcggcggaccttccgtgttc | |
| | | ctgtttcctccaaagcctaaggacaccctgatg | |
| | | atctctcggacccctgaagtgacctgcgtggtg | |
| | | gtggatgtgtctcacgaggacccagaagtgaag | |
| | | ttcaattggtacgtggacggcgtggaagtgcac | |
| | | aacgccaagaccaagcctagagaggaacagtac | |
| | | gccagcacctacagagtggtgtccgtgctgaca | |
| | | gtgctgcaccaggattggctgaacggcaaagag | |
| | | tacaagtgcaaggtgtccaacaaggccctgcct | |
| | | gctcctatcgaaaagaccatcagcaaggccaag | |
| | | ggccagcctagagaaccccaggtttacaccttg | |
| | | cctccatctcgggacgagctgaccaagaaccag | |
| | | gtgtccctgacctgtctcgtgaagggcttctac | |
| | | ccctccgacatcgccgtggaatgggagtctaat | |
| | | ggccagccagagaacaactacaagacaacccct | |
| | | cctgtgctggactccgacggctcattcttcctg | |
| | | tactccaagctgaccgtggacaagtccagatgg | |
| | | cagcagggcaacgtgttctcctgctccgtgatg | |
| | | c | |
| | | acgaggccctgcacaatcactacacacagaagt | |
| | | ccctgtctctgtcccctggc | |
| | Light | gacatccagatgacccagtctccatcctctctg | 69 |
| | Chain | tccgcctctgtgggcgacagagtgaccatcaca | |
| | | tgcaaggccagccaggacatctccaactacctg | |
| | | tcctggttccagcagaagcctggcaaggctccc | |
| | | aagctgctgatctacagagccaacagactggtg | |
| | | gaaggcgtgccctccagattctccggatctggc | |
| | | tctggcaccgactttaccctgacaatctccagc | |
| | | ctgcagcctgaggacttcgctacctacttctgc | |
| | | ctgcaatacgacgagttccctctgacctttggc | |
| | | ggaggcaccaaggtggaaatcaagcggacagtg | |
| | | gccgctccttccgtgttcatcttcccaccttcc | |
| | | gacgagcagctgaagtccggcacagcttctgtc | |
| | | gtgtgcctgctgaacaacttctaccctcgggaa | |
| | | gccaaggtgcagtggaaggtggacaatgccctg | |
| | | cagtccggcaactcccaagagtctgtgaccgag | |
| | | caggactccaaggacagcacctacagcctgtcc | |
| | | tccacactgaccctgtccaaggccgactacgag | |
| | | aagcacaaggtgtacgcctgcgaagtgacccat | |
| | | cagggcctgtctagccctgtgaccaagtctttc | |
| | | aaccggggcgagtgt | |
| B2 | VH | gaagtgcagctggtgcaatctggcgccgaagtg | 70 |
| | | aagaaacctggcgcctctgtgaaggtgtcctgc | |
| | | aaggcctctggcttcaacatcaaggactactac | |
| | | atccactgggtccgacaggctaccggacaggga | |
| | | cttgagtggatgggcagaatcgaccctgaggac | |
| | | gccgagtctgagtacgcccctaagtttcagggc | |
| | | agagtgaccatcaccgccgacacctctaccgac | |
| | | accgcctacatggaactgtccagcctgagatct | |
| | | gaggacaccgccgtgtactactgcaccagaggc | |
| | | gactccatgatggttacctacttcgactactgg | |
| | | ggccagggcaccctggtcacagtttcttcc | |
| | VL | gacatccagatgacccagtctccatcctctctg | 71 |
| | | tccgcctctgtgggcgacagagtgaccatcaca | |
| | | tgcaaggcctctcaggacgtgtccaccgccgtt | |
| | | gcttggtatcagcagaagcctggcaaggcccct | |
| | | aagctgctgatctactgggccttcaccagacac | |
| | | accggcgtgccctctaggttctccggctctggc | |
| | | tctggcaccgattacgctctgacaatctccagc | |
| | | ctgcagcctgaggacttcgccacctactactgc | |
| | | cagcagcactacagaagcccctggacatttggc | |
| | | ggaggcaccaaggtggaaatcaag | |
| | Heavy | gaagtgcagctggtgcaatctggcgccgaagtg | 72 |
| | Chain | aagaaacctggcgcctctgtgaaggtgtcctgc | |
| | | aaggcctctggcttcaacatcaaggactactac | |
| | | atccactgggtccgacaggctaccggacaggga | |
| | | cttgagtggatgggcagaatcgaccctgaggac | |
| | | gccgagtctgagtacgcccctaagtttcagggc | |
| | | agagtgaccatcaccgccgacacctctaccgac | |
| | | accgcctacatggaactgtccagcctgagatct | |
| | | gaggacaccgccgtgtactactgcaccagaggc | |
| | | gactccatgatggttacctacttcgactactgg | |
| | | ggccagggcaccctggtcacagtttcttccgct | |
| | | tccaccaagggacccagcgtgttccctctggct | |
| | | ccttccagcaagtctacctctggcggaacagct | |
| | | gctctgggctgcctggtcaaggattacttccct | |
| | | gagcctgtgaccgtgtcctggaactctggcgct | |
| | | ctgacatccggcgtgcacacctttccagctgtg | |
| | | ctgcaatcctccggcctgtactctctgtcctcc | |
| | | gtcgtgaccgtgccttctagctctctgggcacc | |
| | | cagacctacatctgcaatgtgaaccacaagcct | |
| | | tccaacaccaaggtggacaagaaggtggaaccc | |
| | | aagtcctgcgacaagacccacacctgtcctcca | |
| | | tgtcctgctccagaactgctcggcggaccttcc | |
| | | gtgttcctgtttcctccaaagcctaaggacacc | |
| | | ctgatgatctctcggacccctgaagtgacctgc | |
| | | gtggtggtggatgtgtctcacgaggacccagaa | |
| | | gtgaagttcaattggtacgtggacggcgtggaa | |
| | | gtgcacaacgccaagaccaagcctagagaggaa | |
| | | cagtacgcctccacctacagagtggtgtccgtg | |
| | | ctgacagtgctgcaccaggattggctgaacggc | |
| | | aaagagtacaagtgcaaggtgtccaacaaggca | |
| | | ctgcccgctcctatcgaaaagaccatctccaag | |
| | | gccaagggccagcctagagaaccccaggtttac | |
| | | accttgcctccatctcgggacgagctgaccaag | |
| | | aaccaggtgtccctgacctgtctcgtgaagggc | |
| | | ttctacccctccgacatcgccgtggaatgggag | |
| | | tctaatggccagccagagaacaactacaagaca | |
| | | acccctcctgtgctggactccgacggctcattc | |
| | | ttcctgtactccaagctgacc | |
| | | gtggacaagtccagatggcagcagggcaacgtg | |
| | | ttctcctgctccgtgatgcacgaggccctgcac | |
| | | aatcactacacacagaagtctctgtccctgtct | |
| | | cctggc | |
| | Light | gacatccagatgacccagtctccatcctctctg | 73 |
| | Chain | tccgcctctgtgggcgacagagtgaccatcaca | |
| | | tgcaaggcctctcaggacgtgtccaccgccgtt | |
| | | gcttggtatcagcagaagcctggcaaggcccct | |
| | | aagctgctgatctactgggccttcaccagacac | |
| | | accggcgtgccctctaggttctccggctctggc | |
| | | tctggcaccgattacgctctgacaatctccagc | |
| | | ctgcagcctgaggacttcgccacctactactgc | |
| | | cagcagcactacagaagcccctggacatttggc | |
| | | ggaggcaccaaggtggaaatcaagcggacagtg | |
| | | gccgctccttccgtgttcatcttcccaccttcc | |
| | | gacgagcagctgaagtccggcacagcttctgtc | |
| | | gtgtgcctgctgaacaacttctaccctcgggaa | |
| | | gccaaggtgcagtggaaggtggacaatgccctg | |
| | | cagtccggcaactcccaagagtctgtgaccgag | |
| | | caggactccaaggacagcacctacagcctgtcc | |
| | | tccacactgaccctgtccaaggccgactacgag | |
| | | aagcacaaggtgtacgcctgcgaagtgacccat | |
| | | cagggcctgtctagccctgtgaccaagtctttc | |
| | | aaccggggcgagtgt | |
| C2 | VH | caggtgcagctggtgcagtctggcgccgaagtg | 74 |
| | | aaaaagcctggcgcctctgtgaaggtgtcctgc | |
| | | aaggcctctggcttcaacatcgaggactactac | |
| | | atgcactgggtccgacaggcccctggccagaga | |
| | | ttggaatggatgggcagaatcgaccccgaggac | |
| | | gccgagacaacctactctcctaagttccagggc | |
| | | cgcgtgacaatcatccctgacacctctgccaac | |
| | | accgcctacatggaactgtccagcctgagatct | |
| | | gaggacaccgccgtgtactactgcgcccggtct | |
| | | atctacctggaccctatggactattggggccag | |
| | | ggcaccctggtcacagtgtcctct | |
| | VL | gacatccagatgacccagtctccaaagtctctg | 75 |
| | | tccgcctccgtgggcgacagagtgaccatcacc | |
| | | tgtagagcctctcaggacgtgaccaccgctctg | |
| | | gcttggtatcagcagaagcctggccagtctcct | |
| | | aagctgctgatctactgggccaccaccagacac | |
| | | tctggcgtgccctctagattctccggctctggc | |
| | | tctggcaccgactttaccctgacaatctccagc | |
| | | ctgcagcctgaggacttcgccacctactactgc | |
| | | cagcagcactacagcaccccttacacctttggc | |
| | | cagggcaccaagctggaaatcaag | |
| | Heavy | caggtgcagctggtgcagtctggcgccgaagtg | 76 |
| | Chain | aaaaagcctggcgcctctgtgaaggtgtcctgc | |
| | | aaggcctctggcttcaacatcgaggactactac | |
| | | atgcactgggtccgacaggcccctggccagaga | |
| | | ttggaatggatgggcagaatcgaccccgaggac | |
| | | gccgagacaacctactctcctaagttccagggc | |
| | | cgcgtgacaatcatccctgacacctctgccaac | |
| | | accgcctacatggaactgtccagcctgagatct | |
| | | gaggacaccgccgtgtactactgcgcccggtct | |
| | | atctacctggaccctatggactattggggccag | |
| | | ggcaccctggtcacagtgtcctctgcttctacc | |
| | | aagggacccagcgtgttccctctggctccttcc | |
| | | agcaagtctacctctggcggaacagctgctctg | |
| | | ggctgcctggtcaaggactactttccagagcct | |
| | | gtgaccgtgtcctggaactctggcgctctgaca | |
| | | tctggcgtgcacacctttccagctgtgctgcag | |
| | | tcctccggcctgtactctctgtcctctgtcgtg | |
| | | accgtgccttccagctctctgggaacccagacc | |
| | | tacatctgcaatgtgaaccacaagccttccaac | |
| | | accaaggtggacaagaaggtggaacccaagtcc | |
| | | tgcgacaagacccacacctgtcctccatgtcct | |
| | | gctccagaagctgctggcggcccttccgtgttt | |
| | | ctgttccctccaaagcctaaggacaccctgatg | |
| | | atctctcggacccctgaagtgacctgcgtggtg | |
| | | gtggatgtgtctcacgaggacccagaagtgaag | |
| | | ttcaattggtacgtggacggcgtggaagtgcac | |
| | | aacgccaagaccaagcctagagaggaacagtac | |
| | | aactccacctacagagtggtgtccgtgctgacc | |
| | | gtgctgcaccaggattggctgaacggcaaagag | |
| | | tacaagtgcaaggtgtccaacaaggcactgccc | |
| | | gctcctatcgaaaagaccatctccaaggccaag | |
| | | ggccagcctagggaaccccaggtttacaccctg | |
| | | cctccaagccgggaagagatgaccaagaaccag | |
| | | gtgtccctgacctgcctcgtgaagggcttctac | |
| | | ccttccgacatcgccgtggaatgggagagcaat | |
| | | ggccagccagagaacaactacaagacaacccct | |
| | | cctgtgct | |
| | | ggactccgacggctcattcttcctgtactccaa | |
| | | gctgacagtggacaagtccagatggcagcaggg | |
| | | caacgtgttctcctgctccgtgatgcacgaggc | |
| | | cctgcacaatcactacacacagaagtccctgtc | |
| | | tctgtcccctggc | |
| | Light | gacatccagatgacccagtctccaaagtctctg | 77 |
| | Chain | tccgcctccgtgggcgacagagtgaccatcacc | |
| | | tgtagagcctctcaggacgtgaccaccgctctg | |
| | | gcttggtatcagcagaagcctggccagtctcct | |
| | | aagctgctgatctactgggccaccaccagacac | |
| | | tctggcgtgccctctagattctccggctctggc | |
| | | tctggcaccgactttaccctgacaatctccagc | |
| | | ctgcagcctgaggacttcgccacctactactgc | |
| | | cagcagcactacagcaccccttacacctttggc | |
| | | cagggcaccaagctggaaatcaagcggacagtg | |
| | | gccgctccttccgtgttcatcttcccaccttcc | |
| | | gacgagcagctgaagtccggcacagcttctgtc | |
| | | gtgtgcctgctgaacaacttctaccctcgggaa | |
| | | gccaaggtgcagtggaaggtggacaatgccctg | |
| | | cagtccggcaactcccaagagtctgtgaccgag | |
| | | caggactccaaggacagcacctacagcctgtcc | |
| | | tccacactgaccctgtccaaggccgactacgag | |
| | | aagcacaaggtgtacgcctgcgaagtgacccat | |
| | | cagggcctgtctagccctgtgaccaagtctttc | |
| | | aaccggggcgagtgt | |

In some embodiments, the present disclosure provides an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 54, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 55. In some embodiments, the present disclosure provides an expression vector comprising: an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 54, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 55.

In some embodiments, the present disclosure provides an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 56, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 57. In some embodiments, the present disclosure provides an expression vector comprising: an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 56, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 57.

In some embodiments, the present disclosure provides an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 58, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 59. In some embodiments, the present disclosure provides an expression vector comprising: an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 58, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 59.

In some embodiments, the present disclosure provides an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 60, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 61. In some embodiments, the present disclosure provides an expression vector comprising: an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 60, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 61.

In some embodiments, the present disclosure provides an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 62, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 63. In some embodiments, the present disclosure provides an expression vector comprising: an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 62, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 63.

In some embodiments, the present disclosure provides an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 5, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 32. In some embodiments, the present disclosure provides an expression vector comprising: an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 5, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 32.

In some embodiments, the present disclosure provides an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 79, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 28. In some embodiments, the present disclosure provides an expression vector comprising: an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 79, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 28.

In some embodiments, the present disclosure provides an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 64, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 65. In some embodiments, the present disclosure provides an expression vector comprising: an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 64, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 65.

In some embodiments, the present disclosure provides an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 66, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 67. In some embodiments, the present disclosure provides an expression vector comprising: an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 66, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 67.

In some embodiments, the present disclosure provides an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 68, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 69. In some embodiments, the present disclosure provides an expression vector comprising: an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 68, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 69.

In some embodiments, the present disclosure provides an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 70, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 71. In some embodiments, the present disclosure provides an expression vector comprising: an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 70, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 71.

In some embodiments, the present disclosure provides an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 72, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 73. In some embodiments, the present disclosure provides an expression vector comprising: an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 72, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 73.

In some embodiments, the present disclosure provides an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 74, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 75. In some embodiments, the present disclosure provides an expression vector comprising: an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 74, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 75.

In some embodiments, the present disclosure provides an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 76, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 77. In some embodiments, the present disclosure provides an expression vector comprising: an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 76, and/or an isolated nucleic acid comprising a sequence at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 77.

In some embodiments, the anti-TMPRSS6 described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 54, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 55.

In some embodiments, the anti-TMPRSS6 described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 56, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 57.

In some embodiments, the anti-TMPRSS6 described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 58, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 59.

In some embodiments, the anti-TMPRSS6 described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 60, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 61.

In some embodiments, the anti-TMPRSS6 described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 62, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 63.

In some embodiments, the anti-TMPRSS6 described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 5, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 32.

In some embodiments, the anti-TMPRSS6 described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 79, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 80.

In some embodiments, the anti-TMPRSS6 described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 64, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 65.

In some embodiments, the anti-TMPRSS6 described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 66, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 67.

In some embodiments, the anti-TMPRSS6 described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 68, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 69.

In some embodiments, the anti-TMPRSS6 described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 70, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 71.

In some embodiments, the anti-TMPRSS6 described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 72, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 73.

In some embodiments, the anti-TMPRSS6 described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 74, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 75.

In some embodiments, the anti-TMPRSS6 described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 76, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 77.

In some embodiments, the present disclosure provides a recombinant cell (e.g., a recombinant cell for producing the antibody) expressing the anti-TMPRSS6 antibody described herein.

Accordingly, the present disclosure provides methods for producing the antibody, the methods comprising culturing the recombinant cells under conditions suitable for expression of the antibody from the expression vector by the recombinant cell. Recombinant cells expressing the antibody can be cultured in any suitable condition known in the art. In some embodiments, the method further comprising isolating the antibody from the culture media in which the cell or cells were cultured using any suitable known methods in the art.

### IV. Pharmaceutical Composition

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The anti-TMPRSS6 antibody containing pharmaceutical composition disclosed herein may further comprise a suitable buffer agent. A buffer agent is a weak acid or base used to maintain the pH of a solution near a chosen value after the addition of another acid or base. In some examples, the buffer agent disclosed herein can be a buffer agent capable of maintaining physiological pH despite changes in carbon dioxide concentration (produced by cellular respiration). Exemplary buffer agents include, but are not limited to, HEPES (4-(2-hydroxyethyl)-1-piperazineethanesulfonic acid) buffer, Dulbecco's phosphate-buffered saline (DPBS) buffer, or Phosphate-buffered Saline (PBS) buffer. Such buffers may comprise disodium hydrogen phosphate and sodium chloride, or potassium dihydrogen phosphate and potassium chloride.

In some embodiments, the buffer agent in the pharmaceutical composition described herein may maintain a pH value of about 5-8. For example, the pH of the pharmaceutical composition can be about 7.1, 7.2, 7.3, 7.4, 7.5, 7.6, 7.7, 7.8, 7.9, or 8.0. In other examples, the pharmaceutical composition may have a pH value lower than 7, for example, about 7, 6.8, 6.5, 6.3, 6, 5.8, 5.5, 5.3, or 5.

The pharmaceutical composition described herein comprises one or more suitable salts. A salt is an ionic compound that can be formed by the neutralization reaction of an acid and a base. (Skoog, D. A; West, D. M.; Holler, J. F.; Crouch, S. R. (2004). “chapters 14-16”. Fundamentals of Analytical Chemistry (8th ed.)). Salts are composed of related numbers of cations (positively charged ions) and anions (negative ions) so that the product is electrically neutral (without a net charge).

In some embodiments, the pharmaceutical compositions can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). In some embodiments, the pharmaceutical composition can be formulated for intravenous injection. In some embodiments, the pharmaceutical composition can be formulated for subcutaneous injection.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous or subcutaneous solution bag or vial having a stopper pierceable by a hypodermic injection needle.

### V. Methods of Use

In certain aspects, the disclosure provides methods and related compositions (e.g., anti-TMPRSS6 antibodies) for treating iron overload and conditions associated with iron overload, including, for example, sickle cell disease (SCD), thalassemia (e.g., α-thalassemia, β-thalassemia major, β-thalassemia minor), hemochromatosis (e.g., type 1 hemochromatosis, type 2 hemochromatosis, type 3 hemochromatosis, type 4 hemochromatosis), transfusion related iron overload (e.g., repeated blood transfusion for anemia; large volume transfusion for blood loss from trauma), hemolytic anemia (e.g., transfusion dependent hemolytic anemia, pyruvate kinase deficiency hemolytic anemia), African iron overload, Blackfan-Diamond anemia, refractory anemia with ring-sideroblasts (RARS), myelodysplastic syndrome (MDS) (e.g., SF3B1 associated MDS). In some embodiments, the disclosure provides methods for treating sickle cell disease (SCD). In some embodiments, the disclosure provides methods for treating sickle cell disease in a subject with iron overload.

Aspects of the disclosure relate to methods and compositions (e.g., anti-TMPRSS6 antibodies) useful for treating iron overload. Iron overload occurs when there are excess stores of iron in the body. In some embodiments, iron overload is primary iron overload caused by a hereditary disorder (e.g., hemochromatosis). In some embodiments, iron overload is secondary iron overload caused by a condition that increases iron stores in the body (e.g., transfusion, or hemolysis), or a disorder that impair red blood cell production (i.e., ineffective erythropoiesis) (e.g., sickle cell disease, thalassemia, refractory anemia with ring-sideroblasts (RARS), MDS). Excess iron is deposited into organs throughout the body, which can lead to damage to the organs. Organs that commonly become damaged due to iron deposition are the liver, heart, and endocrine glands.

In some aspects, matriptase-2 regulates iron homeostasis by regulating hepcidin level in a subject. Iron is an essential component for almost all living cells and organisms. Excess iron is hazardous (e.g., due to its redox reactivity that promotes oxidative stress). Thus, dysregulation of iron metabolism leads to diseases. In mammals, the majority of body iron (e.g., >70%) is distributed in red blood cells and mediates oxygen transport within hemoglobin. Senescent red blood cells are cleared by tissue macrophages and iron from senescent red blood cells is recycled to erythroblasts for re-utilization. Iron release into plasma involves ferroportin, a ferrous (Fe²⁺) iron exporter. Following its export, ferrous iron undergoes oxidation to ferric (Fe³⁺) by ceruloplasmin, a circulating ferroxidase, and captured by the iron carrier transferrin. The main function of transferrin is iron delivery to tissues via transferrin receptor 1 (TfR1). Duodenal enterocytes internalize iron from the intestinal lumen via the divalent metal transporter 1 (DMT1), end export it to plasma via ferroportin.

Iron entry into the bloodstream is critical for systemic iron homeostasis and is negatively regulated by hepcidin, an iron regulatory hormone (see, e.g., Ganz T. Systemic iron homeostasis. Physiol Rev. (2013) 93:1721-41). Hepcidin is expressed in hepatocytes as a pre-pro-peptide and undergoes proteolytic processing. Mature hepcidin is a cysteine-rich peptide of 25 amino acids. In some situations, binding of hepcidin to ferroportin in tissue macrophages, duodenal enterocytes and other target cells triggers ubiquitination, internalization and degradation of ferroportin in lysosomes, which leads to iron sequestration within macrophages, inhibition dietary iron absorption, and in turn decreasing of plasma iron levels. Therefore, iron level can be regulated by manipulating hepcidin level in a subject. Expression of hepcidin is regulated by BMP signaling pathway and IL-6-JAK-STAT signaling pathway. Via the BMP pathway, the binding of BMPs (e.g., BMP6 or BMP2) to BMP receptors (e.g., type I (ALK2 and ALK3), type II (ActRIIA and BMPR2) BMP receptors) on the plasma membrane of hepatocytes promotes hepcidin expression. BMP co-receptors, Human homeostatic iron regulator protein (HFE), hemojuvelin (HJV), and transferrin receptor 2 (TFR2), is each required for hepcidin expression by the BMP signaling pathway. The BMP-HJV signaling pathway is negatively regulated by the serine protease matriptase-2 (TMPRSS6), which cleaves and thereby inactivates the type I and II BMP receptors, and HJV (Wahedi et al. Matriptase-2 suppresses hepcidin expression by cleaving multiple components of the hepcidin induction pathway. J Biol Chem. (2017) 292:18354-71). In some embodiments, cleavage of HJV by matriptase-2 reduces the amount of cell surface HJV available to participate in BMP signaling, and in turn decreases hepcidin expression. In some embodiments, loss of hemojuvelin function may be associated with iron overload (e.g., type 2 hemochromatosis). In some embodiments, inhibition of HJV reduces hepcidin expression induced by the IL-6-JAK-STAT signaling pathway. For example, in some embodiments, homozygous HJV knockdown animals fail to amplify hepcidin synthesis in response to IL-6 and are unable to mount an effective hypoferremic response to acute inflammation. Therefore, in some embodiments, inhibition of matriptase-2 (e.g., by an anti-TMPRSS6 antibody) positively regulates hepcidin expression by maintaining/increasing HJV activity. In some embodiments, increased hepcidin level by inhibition of matriptase-2 (e.g., by an anti-TMPRSS6 antibody) decreases iron levels in a subject, thereby treating iron overload.

In some embodiments, methods provided herein comprises administering an effective amount of an anti-TMPRSS6 antibody to a subject for purposes of increasing hepcidin expression. Increased hepcidin level in the subject reduced iron levels. In some embodiments, methods provided herein treat iron overload in a subject by increasing hepcidin level in the subject. In some embodiments, methods provided herein reduce systemic iron. In some embodiments, reduced systemic iron leads to decreased mean corpuscular hemoglobin concentration (MCHC). In some embodiments, the methods provided herein comprise administering an effective amount of an anti-TMPRSS6 antibody to a subject for purposes of treating and/or improving symptoms and conditions associated with iron overload. For example, administering the anti-TMPRSS6 antibody treats and/or improves one or more of the following symptoms: joint pain, fatigue, weight loss, skin discoloration, abdominal pain, hair loss, loss of sex drive, foggy memory, or arrhythmia. In some embodiments, methods provided herein comprises administering an effective amount of an anti-TMPRSS6 antibody to a subject for purposes of improving the symptoms and conditions associated with secondary iron overload (e.g., sickle cell disease, thalassemia, refractory anemia with ring-sideroblasts (RARS), MDS), as described elsewhere herein. In some embodiment, methods provided herein comprises administering an effective amount of an anti-TMPRSS6 antibody to a subject for treating sickle cell disease (SCD), thalassemia (e.g., α-thalassemia, β-thalassemia major, β-thalassemia minor), hemochromatosis (e.g., type 1 hemochromatosis, type 2 hemochromatosis, type 3 hemochromatosis, type 4 hemochromatosis), transfusion related iron overload (e.g., repeated blood transfusion for anemia; large volume transfusion for blood loss from trauma), hemolytic anemia (e.g., transfusion dependent hemolytic anemia, pyruvate kinase deficiency hemolytic anemia), African iron overload, Blackfan Diamond anemia, refractory anemia with ring-sideroblasts (RARS), myelodysplastic syndrome (MDS) (e.g., SF3B1 associated MDS).

Further aspects of the disclosure related to methods and compositions (e.g., anti-TMPRSS6 antibody) useful for treating sickle cell disease (SCD). In some embodiments, the methods provided herein comprise administering an effective amount of an anti-TMPRSS6 antibody to a subject for treating SCD in the subject. In some embodiments, the methods provided herein comprise administering an effective amount of an anti-TMPRSS6 antibody to a subject for purposes of treating SCD in the subject having iron overload. In some embodiments, the methods provided herein reduce iron overload in a subject having SCD. In some embodiments, methods described herein reduces systemic iron level in a subject having SCD. In some embodiments, methods provided herein reduce mean corpuscular hemoglobin concentration (MCHC) in a subject having SCD. In some embodiments, the methods provided herein comprise administering an effective amount of an anti-TMPRSS6 antibody to a subject for purposes of treating and/or improving symptoms and conditions associated with SCD (e.g., vasoocclusive crisis). Sickle cell disease (SCD) is an umbrella term for a group of genetic diseases that affect the hemoglobin gene. SCD is usually caused by an autosomal recessive mutation (e.g., a single nucleotide substitution) in the β-globin gene, which results in the synthesis of sickle hemoglobin (HbS) and sickle-shaped red blood cells (RBCs) (Sundd et al., Pathophysiology of sickle cell disease. Annu Rev Pathol. 2019; 14(1):263-292; Kato G J, et al. Sickle cell disease. Nat Rev Dis Primers. 2018; 4(1):18010). There are several types of sickle disease: Hemoglobin SS Disease, Hemoglobin SC Disease, Hemoglobin SB+ (Beta) Thalassemia, Hemoglobin SB 0 (Beta Zero) Thalassemia, Hemoglobin SD, Hemoglobin SE, And Hemoglobin SO. In hemoglobin SS Disease (i.e., sickle cell anemia), a subject is a homozygous for the hemoglobin S (HbS) gene mutation, and only produces hemoglobin S. In hemoglobin SS disease, the typical disc shape of the RBC is changed into a sickle shape; this deformation of RBCs disrupts its primary functions. Other symptoms of Hemoglobin SS Disease include fatigue, repeated infections, occurrence of periodic pains and internal organ damage. Iron supplements are not considered to be effective in increasing the levels of hemoglobin present in the blood. In hemoglobin SC disease, a subject has one allele of the HbS gene and one allele of the hemoglobin C (HbC) gene. Generally, in hemoglobin SC disease, anemia is less severe than in hemoglobin SS disease. It is likely because hemoglobin C gene doesn't polymerize as rapidly as hemoglobin S. Therefore, it results in the formation of few sickle cells. The symptoms of hemoglobin SS disease and hemoglobin SC disease are similar but differs slightly in severity.

In SCD (e.g., hemoglobin SS disease and/or hemoglobin SC disease), upon deoxygenation in capillaries, HbS polymerizes in rigid fibers, causing RBC membrane distortion that may lead to intravascular hemolysis and inflammation. In some embodiments, the extent of HbS polymerization is related to hemoglobin concentration in the red blood cells. Hemoglobin concentration in the red blood cells can be measured by mean corpuscular hemoglobin concentration (MCHC). MCHC is a measurement of the average amount of hemoglobin in a single red blood cell. In some embodiments, HbS polymerization in SCD patients can be decreased decreasing hemoglobin concentration in the red blood cells (see, e.g., Sunshine et al., Requirements for therapeutic inhibition of sickle haemoglobin gelation, Nature volume 275, pages 238-240 (1978)). In some embodiments, decreasing systemic iron results in decreased HbS polymerization. In some embodiments, decreasing systemic iron leads to decreased MCHC, which results in decreased HbS polymerization (see, e.g., Castro et al., Iron restriction in sickle cell anemia: Time for controlled clinical studies, American Journal of Hematology, Vol. 90, No. 12, December 2015; Brugnara, Less (Fe) is more (Hb) in SCA, Blood (2021) 137 (11): 1446-1447; Embury et al., Concurrent sickle cell anemia and alpha-thalassemia. Effect on pathological properties of sickle erythrocytes, J Clin Invest. 1984 January; 73(1): 116-123; Brewin et al., The pleiotropic effects of α-thalassemia on HbSS and HbSC sickle cell disease: Reduced erythrocyte cation co-transport activity, serum erythropoietin, and transfusion burden, do not translate into increased survival, Am J Hematol. 2022 October; 97(10):1275-1285). Intravascular hemolysis in SCD results in the release of cell-free hemoglobin into circulation, which oxidizes and liberates reactive heme into the vasculature. In some embodiments, release of cell-free hemoglobin into circulation causes iron overload in SCD patients. In some embodiments, cell-free heme activates endothelial cell adhesion molecules and induces leukocyte activation and migration, as well as release of reactive oxygen species, cytokines, and chemokines. In some embodiments, HbS polymerization, hemolysis, release of hemoglobin and/or cell free heme lead to vasoocclusive crisis (VOC). As used herein, the terms vasoocclusive crisis (VOC), vasoocclusion, painful crisis/crises, and vasoocclusive painful crisis/crises are interchangeable with one another. Blood vessel occlusion is a fundamental pathological process in SCD (Frenette P S. Sickle cell vaso-occlusion: multistep and multicellular paradigm. Curr Opin Hematol. (2002) 9:101-6; Pathare et al., Cytokines in sickle cell disease. Hematology. (2003) 8:329-37). During VOC, in some embodiments, the vessel lumen is blocked by cells interrupting the capillary blood flow to various organs and other parts of the body. This precipitates an inflammatory process that leads to painful crises and damage to various tissues (e.g., the brain, the liver, the kidneys, the lungs, the spleen, etc.). Painful crises affect virtually all patients with SCD, often beginning in late infancy and recurring throughout life. In some embodiments, patients with SCD are treated with blood transfusion (e.g., periodic on demand or chronic blood transfusions). In some embodiments, blood transfusion accelerates hemolysis and/or iron overload (e.g., Raghunath et al., Iron Overload in Sickle Cell Disease, Advances in Hematology, Volume 2010|Article ID 272940).

In some embodiments, a subject with SCD has splenic sequestration. The main function of the spleen is the removal of defective RBCs, including sickled RBCs. In patients with SCD, blood flow through the spleen is slow, leading to reduced oxygen tension, and thus increased polymerization of HbS. Due to the narrow capillaries in the splenic vascular bed, the spleen then becomes engorged with blood cells. Splenic sequestration (also referred to as splenic pooling) is characterized by an acutely enlarging spleen (splenomegaly), with hemoglobin more than 2 g/dL below the affected individual's baseline value (Bender and Carlberg, 2003 Sep. 15 [updated 2023 Dec. 28]. In: Adam M P, Feldman J, Mirzaa G M, Pagon R A, Wallace S E, Bean L J H, Gripp K W, Amemiya A, editors. GeneReviews® [Internet]. Seattle (WA): University of Washington, Seattle; 1993-2024.) In some embodiments, a subject with splenic sequestration requires hospitalization, splenectomy, and/or blood transfusion. In some embodiments, administration of an anti-TMPRSS6 antibody reduces splenic sequestration (e.g., frequency and/or severity) in a subject with SCD compared to the subject before administration, or compared to an untreated subject with SCD. In some embodiments, administration of an anti-TMPRSS6 antibody reduces hospitalization (e.g., hospitalization associated with splenectomy) associated with splenic sequestration in a subject with SCD compared to the subject before administration, or compared to an untreated subject with SCD. In some embodiments, administration of an anti-TMPRSS6 antibody reduces the frequency of blood transfusions needed in a subject with SCD compared to the subject before administration, or compared to an untreated subject with SCD. In some embodiments, administration of an anti-TMPRSS6 antibody reduces the size of an enlarged spleen (e.g., a subject with splenomegaly) in a subject with SCD compared to the subject before administration, or compared to an untreated subject with SCD.

In some embodiments, a subject with SCD has extramedullary hematopoiesis (EMH) (see, e.g., Gupta, et al., Clinicopathological characteristics and management of extramedullary hematopoiesis: A review. Pediatric Hematology Oncology Journal 7.4 (2022): 182-186). EMH refers to the production of blood cells outside of the bone marrow, typically occurring in organs such as the liver, spleen, and lymph nodes. In a subject with SCD, EMH may occur due to chronic anemia and the body's compensatory response to maintain adequate blood cell production. In some embodiments, EMH in a subject with SCD occurs due to chronic hemolytic anemia, splenomegaly, ineffective erythropoiesis, VOC, and/or organ damage and dysfunction, all of which contribute to an increased need for blood cell production. In some embodiments, administration of an anti-TMPRSS6 antibody reduces EMH in a subject with SCD compared to the subject before administration, or compared to an untreated subject with SCD.

In some embodiments, a subject with SCD has hepatomegaly (see, e.g., Burley et al., Acute Liver Failure in Sickle Cell Disease: A Perfect Storm. Cureus. 2021 Jun. 16; 13(6):e15680). In some embodiments, hepatomegaly in a subject with SCD is caused by congestion in blood vessels caused by sickled red blood cells, which, in some embodiments, leads to liver infarction of the liver. In some embodiments, the liver becomes enlarged due to congestion caused by blocked blood flow. In some embodiments, hepatomegaly in a subject with SCD is caused by sickle cell crises (e.g., hepatic sequestration). In some embodiments, during a splenic sequestration crisis, blood also becomes trapped in the liver, leading to its enlargement. In some embodiments, hepatomegaly in a subject with SCD is caused by hepatic dysfunction. In some embodiments, hepatic dysfunction in a subject with SCD is caused by chronic hemolysis, iron overload (e.g., from repeated blood transfusions), and/or hepatic congestion. In some embodiments, hepatomegaly in a subject with SCD is caused by gallbladder complications (e.g., gallstones and cholecystitis), which may lead to biliary obstruction and liver enlargement. In some embodiments, hepatomegaly in a subject with SCD is caused by hepatic iron overload (e.g., from chronic transfusion therapy) (e.g., secondary hemochromatosis). Excess iron deposition in the liver may lead to hepatomegaly and liver dysfunction. In some embodiments, hepatomegaly in a subject with SCD is caused, at least in part, by hepatotoxicity related to medications taken for the treatment of SCD (e.g., hydroxyurea), leading to drug-induced liver injury. In some embodiments, administration of an anti-TMPRSS6 antibody reduces hepatomegaly in a subject with SCD compared to the subject before administration, or compared to an untreated subject with SCD. In some embodiments, administration of an anti-TMPRSS6 antibody reduces hepatic sequestration in a subject with SCD compared to before the subject before administration, or compared to an untreated subject with SCD. In some embodiments, a administration of an anti-TMPRSS6 antibody reduces hepatic congestion in a subject with SCD compared to the subject before administration, or compared to an untreated subject with SCD. In some embodiments, administration of an anti-TMPRSS6 antibody reduces gallbladder complications (e.g., gallstones and/or cholecystitis) in a subject with SCD compared to the subject before administration, or compared to an untreated subject with SCD. In some embodiments, administration of an anti-TMPRSS6 antibody reduces hepatic iron overload in a subject with SCD compared to the subject before administration, or compared to an untreated subject with SCD. In some embodiments, administration of an anti-TMPRSS6 antibody reduces hepatic dysfunction in a subject with SCD compared to the subject before administration, or compared to an untreated subject with SCD. In some embodiments, administration of an anti-TMPRSS6 antibody reduces hepatic infarction in a subject with SCD compared to the subject before administration, or compared to an untreated subject with SCD. In some embodiments, administration of an anti-TMPRSS6 antibody reduces hepatic congestion (e.g., biliary obstruction) in a subject with SCD compared to the subject before administration, or compared to an untreated subject with SCD.

In some embodiments, the present disclosure provides methods and compositions (e.g., anti-TMPRSS6 antibodies) useful for reducing VOC in subjects with SCD (e.g., SCD subjects with or without blood transfusion) via iron restriction. In some embodiments, administration of the anti-TMPRSS6 antibodies reduces frequency of VOC in subjects with SCD (e.g., SCD subjects with or without blood transfusion). In some embodiments, administration of the anti-TMPRSS6 antibodies reduces severity of VOC in subjects with SCD (e.g., SCD subjects with or without blood transfusion). In some embodiments, administration of the anti-TMPRSS6 antibody results in increased level of hepcidin thereby decreasing iron in the circulation (e.g., circulatory iron available for HbS synthesis) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody.

In some embodiments, administration of an anti-TMPRSS6 antibody results in decreased circulating iron (e.g., decreased circulating iron by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of an anti-TMPRSS6 antibody results in decreased circulating iron (e.g., decreased circulating iron by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody. Circulating iron level can be evaluated by conventional lab tests, e.g., measurement of iron in serum, transferrin saturation (TSAT), or total iron binding capacity (TIBC).

In some embodiments, administration of an anti-TMPRSS6 antibody results in increased circulating hepcidin-25 level (e.g., increased circulating hepcidin-25 level by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 1.5 times, at least 2 times, at least 3 times, at least 4 times, at least 5 times, at least 10 times, at least 20 times, at least 50 times or at least 100 times) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of an anti-TMPRSS6 antibody results in increased circulating hepcidin-25 level (e.g., increased circulating hepcidin-25 level by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 1.5 times, at least 2 times, at least 3 times, at least 4 times, at least 5 times, at least 10 times, at least 20 times, at least 50 times or at least 100 times) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody.

In some embodiments, administration of an anti-TMPRSS6 antibody results in increased expression of Hamp. In some embodiments, administration of an anti-TMPRSS6 antibody results in increased Hamp expression (e.g., increased Hamp expression by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of an anti-TMPRSS6 antibody results in increased Hamp expression (e.g., decreased bilirubin by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody.

In some embodiments, administration of an anti-TMPRSS6 antibody results in decreased transferrin saturation (TSAT). In some embodiments, administration of an anti-TMPRSS6 antibody results in decreased TSAT (e.g., decreased TSAT by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of an anti-TMPRSS6 antibody results in decreased TSAT (e.g., decreased TSAT by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody.

In some embodiments, administration of an anti-TMPRSS6 antibody results in decreased extramedullary hematopoiesis. In some embodiments, administration of an anti-TMPRSS6 antibody results in decreased extramedullary hematopoiesis (e.g., decreased extramedullary hematopoiesis by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of an anti-TMPRSS6 antibody results in decreased extramedullary hematopoiesis (e.g., decreased extramedullary hematopoiesis by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody. Extramedullary hematopoiesis can be evaluated by suitable known methods, e.g., ratio of spleen weight to body weight, imaging studies (e.g., X-rays, CT, MRI, ultrasound), biopsy, blood tests (e.g., blood cell morphology or levels of blast cells), bone marrow examination (e.g., changes in bone marrow cellularity, composition, and architecture), or erythropoietin levels and reticulocyte count.

In some embodiments, administration of an anti-TMPRSS6 antibody results in decreased hepatomegaly. In some embodiments, administration of an anti-TMPRSS6 antibody results in decreased hepatomegaly (e.g., decreased hepatomegaly by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at anti-TMPRSS6 antibody or a subject having SCD (e.g., SCD subject with or without blood transfusion) but not receiving the anti-TMPRSS6 antibody. In some embodiments, administration of the anti-TMPRSS6 antibody results in decreased hepatomegaly (e.g., decreased hepatomegaly by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody. Hepatomegaly can be evaluated by suitable known methods, e.g., ratio of liver weight to body weight, imaging studies (e.g., CT, MRI, ultrasound, liver elastography), blood tests (e.g., liver function tests, markers of liver injury [alanine transaminase and aspartate transaminase]), and biopsy.

In some embodiments, administration of the anti-TMPRSS6 antibody results in decreased frequency of vasoocclusive crisis (VOC) (e.g., decreased VOC frequency by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of the anti-TMPRSS6 antibody results in decreased frequency of VOC (e.g., decreased VOC frequency by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody.

In some embodiments, administration of the anti-TMPRSS6 antibody results in decreased severity (e.g., hospitalization and/or duration) of vasoocclusive crisis (VOC) (e.g., decreased VOC severity (e.g., hospitalization and/or duration) by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of the anti-TMPRSS6 antibody results in decreased frequency of vasoocclusive crisis (VOC) (e.g., decreasing VOC by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody. In some embodiments, administration of the anti-TMPRSS6 antibody results in decreased severity (e.g., hospitalization and/or duration) of vasoocclusive crisis (VOC) (e.g., decreasing VOC by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody. VOC (e.g., frequency and/or severity) can be evaluated by suitable known methods, such as complete blood count with white blood cell differential, platelet count, reticulocyte count, and comprehensive metabolic panel with liver and kidney function tests. Typical lab findings include acute drop in hemoglobin concentration, increased platelet count, increased reticulocyte count, and elevated serum urea.

In some embodiments, decreased, administration of an anti-TMPRSS6 antibody results in decreases systemic iron. In some embodiments, administration of an anti-TMPRSS6 antibody results in decreased MCHC. In some embodiments, decreased MCHC results in decreased HbS polymerization. In some embodiments, administration of the anti-TMPRSS6 antibody results in decreased MCHC (e.g., decreased MCHC by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at anti-TMPRSS6 antibody or a subject having SCD (e.g., SCD subject with or without blood transfusion) but not receiving the anti-TMPRSS6 antibody. In some embodiments, administration of the anti-TMPRSS6 antibody results in decreased MCHC (e.g., decreased MCHC by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody. In some embodiments, administration of the anti-TMPRSS6 antibody results in decreased HbS polymerization (e.g., decreased HbS polymerization by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of an anti-TMPRSS6 antibody results in decreased HbS polymerization (e.g., decreased HbS polymerization by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody. HbS polymerization can be evaluated by suitable known methods, e.g., solubility sickling test, hemoglobin electrophoresis, isoelectric focusing, or high performance liquid chromatography (see, e.g., Arishi et al., Techniques for the Detection of Sickle Cell Disease: A Review, Micromachines 2021, 12 (5), 519).

In some embodiments, administration of an anti-TMPRSS6 antibody results in decreased sickling of RBCs (e.g., decreased sickling of RBCs by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of an anti-TMPRSS6 antibody results in decreased sickling of RBCs (e.g., decreased sickling of RBCs by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody. Sickling of RBCs can be evaluated by suitable known methods, e.g., blood smear.

In some embodiments, administration of an anti-TMPRSS6 antibody results in decreased hypochromic RBC levels (e.g., decreased hypochromic RBC levels by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of an anti-TMPRSS6 antibody results in decreased hypochromic RBC levels (e.g., hypochromic RBC levels by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody.

In some embodiments, administration of an anti-TMPRSS6 antibody results in decreased microcytic RBC levels (e.g., decreased microcytic RBC levels by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at anti-TMPRSS6 antibody or a subject having SCD (e.g., SCD subject with or without blood transfusion) but not receiving the anti-TMPRSS6 antibody. In some embodiments, administration of an anti-TMPRSS6 antibody results in decreased microcytic RBC levels (e.g., microcytic RBC levels by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody.

In some embodiments, administration of an anti-TMPRSS6 antibody results in decreased hemolysis (e.g., decreased hemolysis by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of an anti-TMPRSS6 antibody results in decreased hemolysis (e.g., decreased hemolysis by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody. Hemolysis can be evaluated by suitable known methods, e.g., lactate dehydrogenase (LDH), unconjugated bilirubin, total bilirubin, direct bilirubin, and haptoglobin tests, reticulocyte count, or total heme and hemopexin (see, e.g., Hemolytic Anemias. ARUP Consult®. Retrieved Mar. 15, 2023, arupconsult.com/content/hemolytic-anemias).

In some embodiments, administration of an anti-TMPRSS6 antibody results in decreased lactate dehydrogenase (LDH). In some embodiments, administration of the anti-TMPRSS6 antibody results in decreased LDH (e.g., decreased LDH by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of the anti-TMPRSS6 antibody results in decreased LDH (e.g., decreased LDH by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody.

In some embodiments, administration of an anti-TMPRSS6 antibody results in decreased bilirubin (e.g., direct bilirubin and/or total bilirubin). In some embodiments, administration of the anti-TMPRSS6 antibody results in decreased bilirubin (e.g., direct bilirubin and/or total bilirubin) (e.g., decreased bilirubin by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of the anti-TMPRSS6 antibody results in decreased bilirubin (e.g., direct bilirubin and/or total bilirubin) (e.g., decreased bilirubin by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody.

In some embodiments, administration of an anti-TMPRSS6 antibody results in decreased inflammation (e.g., systemic inflammation). In some embodiments, administration of an anti-TMPRSS6 antibody results in decreased inflammation (e.g., decreased inflammation by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of the anti-TMPRSS6 antibody results in decreased inflammation (e.g., decreased inflammation by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody. Inflammation can be evaluated by suitable known methods, e.g., blood tests (e.g., levels of C-reactive protein (CRP), erythrocyte sedimentation rate (ESR), white blood cell (WBC) count, neutrophil count, lymphocyte count, levels of proinflammatory cytokines), imaging studies (e.g., CT, MRI, PET scan), and biopsy.

In some embodiments, administration of an anti-TMPRSS6 antibody results in decreased white blood cell (WBC) count. In some embodiments, administration of an anti-TMPRSS6 antibody results in decreased WBC count (e.g., decreased WBC count by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of the anti-TMPRSS6 antibody results in decreased WBC count (e.g., decreased WBC count by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody.

In some embodiments, administration of an anti-TMPRSS6 antibody results in decreased neutrophil count. In some embodiments, administration of an anti-TMPRSS6 antibody results in decreased neutrophil count (e.g., decreased neutrophil count by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of the anti-TMPRSS6 antibody results in decreased neutrophil count (e.g., decreased neutrophil count by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody.

In some embodiments, administration of an anti-TMPRSS6 antibody results in decreased lymphocyte count. In some embodiments, administration of an anti-TMPRSS6 antibody results in decreased lymphocyte count (e.g., decreased lymphocyte count by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of the anti-TMPRSS6 antibody results in decreased lymphocyte count (e.g., decreased lymphocyte count by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody.

In some embodiments, administration of an anti-TMPRSS6 antibody results in decreased blood transfusion frequency (e.g., decreased blood transfusion frequency by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., SCD subject with or without blood transfusion) relative to the subject prior to receiving the anti-TMPRSS6 antibody or a subject having SCD (e.g., SCD subject with or without blood transfusion) but not receiving the anti-TMPRSS6 antibody. In some embodiments, administration of an anti-TMPRSS6 antibody results in decreased blood transfusion sessions (e.g., decreased blood transfusion sessions by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having SCD (e.g., hemoglobin SC disease) relative to the subject prior to receiving the anti-TMPRSS6 antibody or a subject having SCD (e.g., hemoglobin SC disease) but not receiving the anti-TMPRSS6 antibody.

In some embodiment, the present disclosure provides method of treating a subjecting having SCD by administering the subject an effective amount of the anti-TMPRSS6 antibody in combination with any known therapeutics for treating SCD, e.g., hemoglobin S polymerization inhibitor (e.g., voxelotor), therapeutic agents for reducing VOC (e.g., Hydroxyurea, L-glutamine oral powder, Crizanlizumab, selective pyruvate kinase-R (PKR) activator), pain-relieving medications (e.g., narcotics, opioids, gabapentin, cannabis), blood transfusion, stem cell transplant, Exagmglogene autotemcel (exa-cel), LentiGlobin, GMI-1070, VIT-2763 (vamifeport), Ticagrelor, Vitamin D, simvastatin, AG-348 (mitapivat sulfate), propranolol, epinephrine, regadenoson, atorvastatin, prasugrel, L-arginine, OTQ923, oxygen therapy, and gene therapy.

Further aspects of the disclosure relate to methods and compositions (e.g., anti-TMPRSS6 antibodies) useful for treating thalassemia (e.g., α-thalassemia, β-thalassemia major, or β-thalassemia minor) and/or one or more conditions arising as a result of thalassemia in a subject. In some embodiments, the methods provided herein comprise administering an effective amount of an anti-TMPRSS6 antibody to a subject for purposes of treating thalassemia (e.g., α-thalassemia, β-thalassemia major, or β-thalassemia minor) in a subject having iron overload. In some embodiments, the methods provided herein reduce iron overload in a subject having thalassemia (e.g., α-thalassemia, β-thalassemia major, or β-thalassemia minor). “Thalassemia” comprises a group of inherited hematologic disorders caused by defects in the synthesis of one or more hemoglobin chains. In some embodiments, α-thalassemia is caused by reduced or absent synthesis of alpha globin chains. In some embodiments, β-thalassemia is caused by reduced or absent synthesis of beta globin chains. In some embodiments, imbalances of hemoglobin chains cause hemolysis and impair erythropoiesis.

α-thalassemia is the result of deficient or absent synthesis of alpha globin chains, leading to excess beta globin chains. Alpha globin chain production is controlled by two genes on each chromosome 16 (i.e., four alpha globin genes), and deficient production is usually caused by a deletion of one or more of these genes (Farashi et al., Molecular basis of α-thalassemia. Blood Cells Mol Dis 2018; 70:43-53). A single gene deletion results in α-thalassemia silent carrier status, which is asymptomatic with normal hematologic findings. The two-gene deletion causes α-thalassemia minor with microcytosis and usually no anemia. The three-gene deletion results in significant production of hemoglobin H (HbH), which has four beta chains. α-thalassemia intermedia, or HbH disease, causes microcytic anemia, hemolysis, and splenomegaly. The four-gene deletion results in significant production of hemoglobin Bart's (Hb Bart's), which has four gamma chains. α-thalassemia major with Hb Bart's usually results in fatal hydrops fetalis (Harteveld and Higgs, α-thalassemia. Orphanet J Rare Dis 2010; 5:13).

β-thalassemia is the result of deficient or absent synthesis of beta globin chains, leading to excess alpha chains, which in turn results in impaired erythropoiesis. Beta globin chain production is controlled by the beta globin gene on chromosome 11. Beta globin chain production can range from near normal to completely absent, leading to varying degrees of excess alpha globin to beta globin chain production. Three main forms have been described: β-thalassemia major (also known as transfusion dependent β-thalassemia or Cooley's anemia), β-thalassemia intermedia and β-thalassemia minor (Origa, β-Thalassemia. Genet Med 2017; 19(6):609-619). Individuals with β-thalassemia major have two missing or defective beta globin genes, resulting in absent of beta globin chains. Subjects with β-thalassemia major usually present (e.g., within the first two years of life) with severe anemia, requiring regular red blood cell (RBC) transfusions. Findings in untreated or poorly transfused individuals with β-thalassemia major can include hemolysis, growth retardation, pallor, jaundice, poor musculature, hepatosplenomegaly, leg ulcers, development of masses from extramedullary hematopoiesis, and skeletal changes that result from expansion of the bone marrow. In some embodiments, treatment options for β-thalassemia major includes regular blood transfusions, iron chelation and management of secondary complications of iron overload. In some circumstances, spleen removal may be required, and bone marrow transplantation remains the only definitive cure currently available. Regular transfusion therapy leads to iron overload-related complications including endocrine complication (growth retardation, failure of sexual maturation, diabetes mellitus, and insufficiency of the parathyroid, thyroid, pituitary, and less commonly, adrenal glands), dilated myocardiopathy, liver fibrosis and cirrhosis).

Patients with β-thalassemia intermedia, which is caused by two missing or defective beta globin genes with significant genetic heterogeneity and clinical polymorphisms, present later in life with moderate anemia and do not require regular transfusions. Main clinical features in these patients are hypertrophy of erythroid marrow with medullary and extramedullary hematopoiesis and its complications (osteoporosis, masses of erythropoietic tissue that primarily affect the spleen, liver, lymph nodes, chest and spine, and bone deformities and typical facial changes), hemolysis gallstones, iron accumulation, painful leg ulcers and increased predisposition to thrombosis. In some embodiments, subjects with β-thalassemia intermedia can undergo blood transfusion if the subject is having severe and persistent symptoms (e.g., persistent leg ulcer and pain associated with the leg ulcer, pulmonary hypertension, severe anemia, failure of growth) (see, e.g., Cappellini et al., Guidelines for the Clinical Management of Thalassaemia, 2nd Revised edition, Chapter 11, Thalassaemia Intermedia and HbE, Nicosia (CY), Thalassaemia International Federation; 2008, ISBN-13:978-9963-623-70-9).

In β-thalassemia minor, only one beta globin gene is damaged or missing. Subjects with β-thalassemia minor are mildly anemic, hypochromic, and microcytic (i.e., have increased levels of hypochromic and/or microcytic red blood cells, respectively). In some embodiments, iron overload occurs in subjects having β-thalassemia minor and are supplemented with iron for their anemia.

In some embodiments, iron overload is present in subjects having thalassemia (e.g., all forms of α-thalassemia and β-thalassemia). In some embodiments, iron overload is associated with increased morbidity in subjects with thalassemia (e.g., all forms of α-thalassemia and β-thalassemia). In some embodiments, iron load in thalassemia subjects are secondary to blood transfusion (e.g., in β-thalassemia major and β-thalassemia intermedia). In some embodiments, iron overload in beta thalassemia subjects (e.g., mainly β-thalassemia major and β-thalassemia intermedia) is secondary to ineffective erythropoiesis characterized by enhanced intestinal iron absorption mediated by hepcidin suppression (see, e.g., Gardenghi et al., Ineffective erythropoiesis in β-thalassemia is characterized by increased iron absorption mediated by down-regulation of hepcidin and up-regulation of ferroportin, Blood. 2007 Jun. 1; 109(11): 5027-5035). In some embodiments, suppression of hepcidin expression enhances iron absorption from the intestine and to allow iron release from macrophages, thereby further increases iron levels. Different organs are affected differently by iron overload in different forms of thalassemia owing to the underlying iron loading mechanism and/or rate of iron accumulation (see, e.g., Taher and Saliba, Iron overload in thalassemia: different organs at different rates. Hematology Am Soc Hematol Educ Program 2017; 2017(1):265-271). In some embodiments, the methods described herein treats thalassemia (e.g., α-thalassemia, β-thalassemia major, or β-thalassemia minor) in a subject. In some embodiments, the thalassemia is not β-thalassemia intermedia. In some embodiments, the present disclosure provides methods for treating iron overload in subjects having thalassemia (e.g., α-thalassemia, β-thalassemia major, or β-thalassemia minor) by administering the subject an anti-TMPRSS6 antibody. In some embodiments, an anti-TMPRSS6 antibodies disclosed herein inhibits matriptase-2 activity, subsequently increasing hemojuvelin (HJV) levels, leading to increased levels of hepcidin. Subsequently, in some embodiments, hepcidin in turn acts to inhibit iron absorption, release, and recycling, thereby decreasing iron levels. In some embodiments, the methods herein reduce or improves one or more symptoms associated with thalassemia. In some embodiments, the methods herein reduce symptoms associated with iron overload in subjects with thalassemia (e.g., joint pain, abdominal pain, fatigue, weakness, diabetes, heart failure, and/or liver failure).

In some embodiments, administration of an anti-TMPRSS6 antibody results in increased circulating hepcidin-25 level (e.g., increasing circulating hepcidin-25 level by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 1.5 times, at least 2 times, at least 3 times, at least 4 times, at least 5 times, at least 10 times, at least 20 times, at least 50 times or at least 100 times) in a subject having thalassemia (e.g., α-thalassemia, β-thalassemia major, or β-thalassemia minor) relative to the subject prior to receiving the anti-TMPRSS6 antibody or a subject having thalassemia (e.g., α-thalassemia, β-thalassemia major, or β-thalassemia minor) but not receiving the anti-TMPRSS6 antibody.

In some embodiments, administration of an anti-TMPRSS6 antibody results in decreased hemolysis (e.g., decreased hemolysis by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having thalassemia (e.g., α-thalassemia, β-thalassemia major, or β-thalassemia minor) relative to the subject prior to receiving the anti-TMPRSS6 antibody or a subject having thalassemia (e.g., α-thalassemia, β-thalassemia major, or β-thalassemia minor) but not receiving the anti-TMPRSS6 antibody. Hemolysis can be evaluated by suitable known methods described elsewhere herein.

In some embodiments, administration of an anti-TMPRSS6 antibody results in decreased blood transfusion sessions (e.g., decreased blood transfusion sessions by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) in a subject having thalassemia (e.g., α-thalassemia major, α-thalassemia intermedia, β-thalassemia major) relative to the subject prior to receiving the anti-TMPRSS6 antibody or a subject having thalassemia (e.g., α-thalassemia major, α-thalassemia intermedia, β-thalassemia major) but not receiving the anti-TMPRSS6 antibody.

In some embodiment, the present disclosure provides method of treating a subjecting having thalassemia by administering the subject an effective amount of the anti-TMPRSS6 antibody in combination with any known therapeutics for treating thalassemia, e.g., blood transfusion, iron chelation, folic acid supplement, bow marrow transplantation, stem cell transplantation, deferiprone, Luspatercept, splenectomy, or gene therapy, etc.

Further aspects of the disclosure relate to methods and compositions (e.g., anti-TMPRSS6 antibodies) useful for treating hereditary hemochromatosis (HH). In some embodiments, the methods provided herein comprise administering an effective amount of an anti-TMPRSS6 antibody to a subject for treating HH in the subject. In some embodiments, the methods provided herein comprise administering an effective amount of an anti-TMPRSS6 antibody to a subject for purposes of treating HH in the subject having iron overload. In some embodiments, the methods provided herein reduce iron overload in a subject having HH. In some embodiments, the methods provided herein comprise administering an effective amount of an anti-TMPRSS6 antibody to a subject for purposes of treating and/or improving symptoms and conditions associated with HH. Hereditary hemochromatosis (HH) includes a number of inherited disorders that can lead to progressive iron loading. There are 4 main types of HH that have been categorized based on which proteins involved in iron homeostasis are affected (see, e.g., Kowdley et al., ACG Clinical Guideline: Hereditary Hemochromatosis, The American Journal of Gastroenterology 114(8):p 1202-1218 August 2019). Type 1 HH is the most frequent inherited form of iron overload. In some embodiments, type 1 HH is a result of a G to A transition at nucleotide 845 of the HFE gene, resulting in a cysteine to tyrosine substitution at amino acid 282 (C282Y), referred to as type 1a HH. In other embodiments, type 1 HH is a result of a H63D mutation. In some embodiments, subjects with H63D mutation of the HFE gene are not at risk of developing clinically significant iron overload (see, e.g., Gochee et al., A population-based study of the biochemical and clinical expression of the H63D hemochromatosis mutation, Gastroenterology. 2002 March; 122(3):646-51). In some embodiments, type 1 HH is a result of C282Y/H63D substitutions, which is classified as HH type 1b. In some embodiments, subjects with HH type 1b rarely develop clinically significant iron overload unless cofactors such as alcohol or hepatitis C virus (HCV) are involved (see, e.g., Alissa et al., The Clinical Relevance of Compound Heterozygosity for the C282Y and H63D Substitutions in Hemochromatosis, Clinical Gastroenterology & Hepatology, November 2006, Volume 4 (11), 1403-1410). In some embodiments, type 1 HH is a result of an S65C mutation of the HFE gene, and is referred to as type 1c HH. Generally, the S65C mutation is considered a polymorphism without clinical significance.

Type 2 HH, also called juvenile hemochromatosis, is associated with mutations in the HJV gene (type 2A) or the hepatic antimicrobial protein (HAMP) gene (type 2B), respectively, leading to hepcidin deficiency (Papanikolaou et al., Mutations in HFE2 cause iron overload in chromosome 1q-linked juvenile hemochromatosis, Nat Genet. 2004 January; 36(1):77-82). In some embodiments, type 2 HH tends to lead to the most severe form of primary iron overload. In some embodiments, type 2 HH primarily occurs in younger individuals.

Type 3 HH is associated with mutations in the transferrin receptor 2 (TFR2) gene, also leading to hepcidin deficiency (Camaschella et al., The gene TFR2 is mutated in a new type of haemochromatosis mapping to 7q22, Nat Genet. 2000 May; 25(1):14-5). In some embodiments, mutations in TFR2 gene cause HH through a reduction in hepcidin transcription (see, e.g., Christal et al., The role of hepatic transferrin receptor 2 in the regulation of iron homeostasis in the body, Front Pharmacol. 2014; 5:34). In some embodiments, iron overload is present in type 3 HH but is much less severe than type 2 HH.

Type 4 HH is a result of mutations in the ferroportin 1 (FPN1 gene). Type 4A HH, also known as FPN disease, is an autosomal dominant form of hemochromatosis due to mutations in the FPN1 gene (SLC40A1) (Abboud et al., novel mammalian iron-regulated protein involved in intracellular iron metabolism, J Biol Chem. 2000 Jun. 30; 275(26):19906-12). In type 4A HH, the production of hepcidin is normal, but the export function of FPN1 is diminished, leading to intracellular iron retention with low levels of plasma iron and normal or low levels of transferrin saturation but elevated serum ferritin levels (33). The spleen is the most affected organ in type 4A HH, because of high FPN1 activity at the level of macrophages. Type 4B HH is a form of iron overload due to resistance of FPN1 to hepcidin.

In some embodiments, the methods provided herein comprise administering an effective amount of an anti-TMPRSS6 antibody to a subject for treating HH in the subject. In some embodiments, the methods provided herein comprise administering an effective amount of an anti-TMPRSS6 antibody to a subject for purposes of treating the subject with hereditary hemochromatosis (e.g., type 1 HH, type 2, type 3 HH, or type 4 HH) having iron overload. In some embodiments, an anti-TMPRSS6 antibodies disclosed herein inhibits matriptase-2 activity, subsequently increasing hemojuvelin (HJV) levels, leading to increased levels of hepcidin. Subsequently, in some embodiments, hepcidin in turn acts to inhibit iron absorption, release, and recycling, thereby decreasing iron levels, thereby reducing iron overload in subjects having hereditary hemochromatosis (e.g., type 1 HH, type 2, type 3 HH, or type 4 HH). In some embodiments, the methods herein reduce or improve one or more symptoms associated with hereditary hemochromatosis (e.g., type 1 HH, type 2, type 3 HH, or type 4 HH). In some embodiments, the methods herein reduce symptoms associated with iron overload in subjects with hereditary hemochromatosis (e.g., type 1 HH, type 2, type 3 HH, or type 4 HH), such as joint pain, abdominal pain, fatigue, weakness, diabetes, loss of sex drive, impotence, heart failure, liver failure, bronze or gray skin color, and memory fog.

In some embodiment, the present disclosure provides method of treating iron overload in a subjecting having hereditary hemochromatosis (e.g., type I HH, or type 3 HH) by administering the subject an effective amount of the anti-TMPRSS6 antibody in combination with any known therapeutics for treating hereditary hemochromatosis (e.g., type 1 HH, type 2, type 3 HH, or type 4 HH), e.g., phlebotomy, iron chelation (e.g., deferoxamine or deferasirox), supportive care for complications, etc.

Further aspects of the disclosure relate to methods and compositions (e.g., anti-TMPRSS6 antibodies) useful for treating Myelodysplastic syndromes (MDS). In some embodiments, the methods provided herein comprise administering an effective amount of an anti-TMPRSS6 antibody to a subject for treating MDS in the subject. In some embodiments, the methods provided herein comprise administering an effective amount of an anti-TMPRSS6 antibody to a subject for purposes of treating MDS in the subject having iron overload. In some embodiments, the methods provided herein reduce iron overload in a subject having MDS. In some embodiments, the methods provided herein comprise administering an effective amount of an anti-TMPRSS6 antibody to a subject for purposes of treating and/or improving symptoms and conditions associated with MDS. Myelodysplastic syndromes (MDS) are a heterogeneous group of hematologic neoplasms varying in severity affecting one or more lines of hematopoiesis. In some embodiments, ineffective erythropoiesis results in dysregulation of iron metabolism. In some embodiments, iron overload starts to develop in MDS patients before they become transfusion-dependent because ineffective erythropoiesis suppresses hepcidin production in the liver and thus leads to unrestrained intestinal iron uptake. In some embodiments, ineffective erythropoiesis, a hallmark of MDS, causes a massive expansion of bone marrow erythroblasts due to decreased production of mature RBCs (Sebastiani G et al., Pharmacological targeting of the hepcidin/ferroportin axis. Frontiers in Pharmacology. 2016; 7:160). This creates a high demand for iron, leading to the suppression of hepcidin. In some embodiments, hepcidin levels is heterogeneous across different MDS subtypes. In some embodiments, the lowest hepcidin levels were observed in refractory anemia with ringed sideroblasts (RARS). In some embodiments, RARS patients have high levels of toxic non-transferrin bound iron relative to patients having other types of MDS with higher hepcidin levels. In addition, and in some embodiments, tissue hypoxia resulting from ineffective erythropoiesis, triggers increased erythropoietin production, which then results in low hepcidin, and in turn iron overload (Cui et al., Serum iron metabolism and erythropoiesis in patients with myelodysplastic syndrome not receiving RBC transfusions. Leukemia research. 2014; 38:545-550). Accordingly, in some embodiments, the present disclosure provides methods and compositions for treating refractory anemia with ringed sideroblasts (RARS). In some embodiments, the methods provided herein comprise administering an effective amount of an anti-TMPRSS6 antibody to a subject for purposes of treating and/or improving symptoms and conditions associated with RARS. In some embodiments, the methods provided herein comprise administering an effective amount of an anti-TMPRSS6 antibody to a subject for purposes of reducing iron overload associated with RARS. In some embodiments, administration of an anti-TMPRSS6 antibody results in increased hepcidin level in subjects with MDS (e.g., RARS). In some embodiments, administration of an anti-TMPRSS6 antibody results in reduced iron overload in subjects with MDS (e.g., RARS).

In some embodiments, a subject with MDS has a mutation in SF3B1 gene. SF3B1 is a gene encoding a component of the RNA splicing machinery, and mutations in this gene have been found in various types of MDS, including RARS. In some embodiments, MDS patients having SF3B1 mutation has a significantly lower hepcidin to ferritin ratio as compared with those without the mutation (Ilaria et al., Inappropriately low hepcidin levels in patients with myelodysplastic syndrome carrying a somatic mutation of SF3B1. Haematologica. 2013; 98:420-423). In some embodiments, ineffective erythropoietic activity in MDS patients with SF3B1 mutation leads to low hepcidin levels in MDS-RARS patients with an SF3B1 mutation, leading ultimately to excess release of iron from the RES and parenchymal iron loading. Accordingly, in some embodiments, the present disclosure provides methods and compositions for treating MDS with SF3B1 mutation. In some embodiments, the methods provided herein comprise administering an effective amount of an anti-TMPRSS6 antibody to a subject for purposes of treating and/or improving symptoms and conditions associated with MDS with SF3B1 mutation. In some embodiments, the methods provided herein comprise administering an effective amount of an anti-TMPRSS6 antibody to a subject for purposes of reducing iron overload associated with MDS with SF3B1 mutation. In some embodiments, administration of an anti-TMPRSS6 antibody result in increases hepcidin level in subjects with MDS (e.g., MDS with SF3B1 mutation). In some embodiments, administration of an anti-TMPRSS6 antibody result in reduced iron overload in subjects with MDS (e.g., MDS with SF3B1 mutation).

In some embodiments, RARS patients have a high occurrence of SF3B1 mutation (see, e.g., Zhu et al., SF3B1-mutated myelodysplastic syndrome with ring sideroblasts harbors more severe iron overload and corresponding over-erythropoiesis. Leukemia research. 2016; 44:8-16). In some embodiments, as compared with the wild type RARS patients without the SF3B1 mutation, hepcidin is lower in RARS subjects with SF3B1 mutation. In some embodiments, the iron overload is more severe in RARS subjects having SF3B1 mutation as compared to RARS patients without the SF3B1 mutation. Accordingly, in some embodiments, the present disclosure provides methods and compositions for treating RARS with SF3B1 mutation. In some embodiments, the methods provided herein comprise administering an effective amount of an anti-TMPRSS6 antibody to a subject for purposes of treating and/or improving symptoms and conditions associated with RARS with SF3B1 mutation. In some embodiments, the methods provided herein comprise administering an effective amount of an anti-TMPRSS6 antibody to a subject for purposes of reducing iron overload associated with RARS with SF3B1 mutation. In some embodiments, administration of an anti-TMPRSS6 antibody results in increased hepcidin levels in subjects with MDS (e.g., RARS with SF3B1 mutation). In some embodiments, administration of an anti-TMPRSS6 antibody result in reduced iron overload in subjects with MDS (e.g., RARS with SF3B1 mutation).

In addition, in some embodiments, MDS patients have anemia, and some require regular red blood cell transfusions. Chronic transfusion therapy is also a major cause of iron overload in patients having MDS. In some embodiments, MDS patients with iron overload have reduced overall survival and poorer outcomes following allogeneic stem cell transplant vs. those without iron overload (see, e.g., Gattermann, Iron overload in myelodysplastic syndromes (MDS), International Journal of Hematology volume 107, pages 55-63 (2018); Lyle et al., Iron Overload in Myelodysplastic Syndromes: Pathophysiology, Consequences, Diagnosis, and Treatment, J Adv Pract Oncol. 2018; 9 (4): 392-405). Therefore, in some embodiments, the method provided herein comprises administering an effective amount of an anti-TMPRSS6 antibody to a subject for purposes of reducing iron overload associated with blood transfusion in MDS patients.

In some embodiments, the methods provided herein improves symptoms and/or clinical outcome associated with iron overload in subjects with MDS (e.g., RARS, MDS with SF3B1, RARS with SF3B1), such as survival, cardiovascular function, liver function, and immune function.

In some embodiment, the present disclosure provides method of treating a subjecting having MDS (e.g., RARS, MDS with SF3B1, RARS with SF3B1) by administering the subject an effective amount of the anti-TMPRSS6 antibody in combination with any known therapeutics for treating MDS (e.g., RARS, MDS with SF3B1, RARS with SF3B1), e.g., iron chelation, bone marrow transplantation, stem cell transplantation, blood transfusion, EPO, luspatercept, granulocyte colony-stimulating factor (GCSF), antithymocyte globulin (ATG), cyclosporine (CSA), hypomethylating agent (HMA), lenalidomide (LEN), thrombopoietin receptor agonist (TPO-RA), etc.

Further aspects of the disclosure relate to methods and compositions (e.g., anti-TMPRSS6 antibodies) useful for treating hemolytic anemia (e.g., transfusion dependent hemolytic anemia, pyruvate kinase deficiency hemolytic anemia). In some embodiments, hemolysis causes a massive outflow of hemoglobin into the bloodstream, and in turn causes iron overload. In some embodiments, hemolysis induces EPO production to activate erythropoiesis (e.g., without ineffective erythropoiesis). In some embodiments, increased EPO and erythropoiesis repress hepcidin expression, thereby including iron overload in subjects with hemolytic anemia. Accordingly, in some embodiments, the present disclosure provides methods and compositions for treating hemolytic anemia (e.g., transfusion dependent hemolytic anemia, pyruvate kinase deficiency hemolytic anemia). In some embodiments, the methods provided herein comprise administering an effective amount of an anti-TMPRSS6 antibody to a subject for treating hemolytic anemia (e.g., transfusion dependent hemolytic anemia, pyruvate kinase deficiency hemolytic anemia). In some embodiments, the methods provided herein comprise administering an effective amount of an anti-TMPRSS6 antibody to a subject for purposes of treating hemolytic anemia in the subject having iron overload. In some embodiments, the methods provided herein reduces iron overload in a subject having hemolytic anemia. In some embodiments, the methods provided herein comprise administering an effective amount of an anti-TMPRSS6 antibody to a subject for purposes of treating and/or improving symptoms and conditions associated with hemolytic anemia (e.g., transfusion dependent hemolytic anemia, pyruvate kinase deficiency hemolytic anemia). In some embodiments, the methods provided herein comprise administering an effective amount of an anti-TMPRSS6 antibody to a subject for purposes of reducing iron overload associated with hemolytic anemia (e.g., transfusion dependent hemolytic anemia, pyruvate kinase deficiency hemolytic anemia). In some embodiments, administration of an anti-TMPRSS6 antibody result in increases hepcidin level in subjects with hemolytic anemia (e.g., transfusion dependent hemolytic anemia, pyruvate kinase deficiency hemolytic anemia). In some embodiments, administration of an anti-TMPRSS6 antibody result in reduced iron overload in subjects with hemolytic anemia (e.g., transfusion dependent hemolytic anemia, pyruvate kinase deficiency hemolytic anemia).

Further aspects of the disclosure relate to methods and compositions (e.g., anti-TMPRSS6 antibodies) useful for treating transfusion related iron overload. Blood transfusion has been widely used and sometimes overused in medical practice since early 20th century to treat various conditions (e.g., anemia and/or hemorrhage). Transfused red blood cells (RBCs) provide beneficial effects to subjects receiving the transfusion, e.g., increases blood volume, decreases blood viscosity, and increases oxygen carrying capacity. In some embodiments, blood transfusion (e.g., large volume blood transfusion) is used in subjects with blood loss (e.g., from trauma or surgery). In some embodiments, blood transfusion (e.g., repeated blood transfusion) is used in subjects having anemia (e.g., SCD, thalassemia, hemochromatosis, MDS, iron deficiency anemia, Diamond-Blackfan anemia, etc.). In some embodiments, with blood transfusion, a subject receives excess iron. In some embodiments, the excess iron from blood transfusion is stored in various tissues, causing iron overload. In addition, and in some embodiments, blood transfusion causes hemolysis, thereby releasing iron into circulation, further causing iron overload. In some embodiments, Accordingly, in some embodiments, the present disclosure provides methods and compositions for treating iron overload associated with blood transfusion (e.g., blood transfusion for anemia and/or hemorrhage). In some embodiments, the methods provided herein comprise administering an effective amount of an anti-TMPRSS6 antibody to a subject for treating iron overload associated with blood transfusion (e.g., blood transfusion for anemia and/or hemorrhage). In some embodiments, the methods provided herein reduces iron overload in a subject having hemolytic anemia. In some embodiments, the methods provided herein comprise administering an effective amount of an anti-TMPRSS6 antibody to a subject for purposes of reducing iron overload associated with iron overload associated with blood transfusion (e.g., blood transfusion for anemia and/or hemorrhage). In some embodiments, administration of an anti-TMPRSS6 antibody result in increases hepcidin level in subjects having iron overload associated with blood transfusion (e.g., blood transfusion for anemia and/or hemorrhage). In some embodiments, administration of an anti-TMPRSS6 antibody result in reduced iron overload in subjects with iron overload associated with blood transfusion (e.g., blood transfusion for anemia and/or hemorrhage).

Further aspects of the disclosure relate to methods and compositions (e.g., anti-TMPRSS6 antibodies) useful for treating African iron overload. In some embodiments, the methods provided herein comprise administering an effective amount of an anti-TMPRSS6 antibody to a subject for African iron overload. In some embodiments, the methods provided herein reduces iron overload in a subject having African iron overload.\African iron overload is an iron overload resulting from a diet high in iron, and genetic factors. In some embodiments, administration of an anti-TMPRSS6 antibody result in increases hepcidin level in subjects having African iron overload. In some embodiments, administration of an anti-TMPRSS6 antibody result in reduced iron overload in subjects with African iron overload.

Further aspects of the disclosure relate to methods and compositions (e.g., anti-TMPRSS6 antibodies) useful for treating Diamond-Blackfan anemia. In some embodiments, the methods provided herein comprise administering an effective amount of an anti-TMPRSS6 antibody to a subject for treating Diamond-Blackfan anemia. In some embodiments, the methods provided herein comprise administering an effective amount of an anti-TMPRSS6 antibody to a subject for purposes of treating Diamond-Blackfan anemia in the subject having iron overload. In some embodiments, the methods provided herein reduces iron overload in the subject. In some embodiments, the methods provided herein comprise administering an effective amount of an anti-TMPRSS6 antibody to a subject for purposes of treating symptoms and conditions associated with Diamond-Blackfan anemia. Diamond-Blackfan anemia (DBA) is an inherited blood disorder that affects the ability of the bone marrow to produce red blood cells. In some embodiments, Diamond-Blackfan anemia is caused by genetic changes in several genes, including but are not limited to: RPS19, RPL5, RPS10, RPL11, RPL35A, RPS7, RPS17, RPS24, RPS26 and GATA1 genes. In some embodiments, due to the inability to produce red blood cells, patients having DBA is dependent on blood transfusion (see, e.g., Roggero et al., Severe iron overload in Blackfan-Diamond anemia: a case-control study, Am J Hematol. 2009 November; 84(11):729-32; Quarello et al., Diamond-Blackfan anaemia with iron overload: A serious issue, 2022 October; 199(2):171-172). As described elsewhere herein, repeated blood transfusion results in iron overload. Accordingly, in some embodiments, the methods provided herein comprise administering an effective amount of an anti-TMPRSS6 antibody to a subject for purposes of reducing iron overload associated with blood transfusion in a subject having Diamond-Blackfan anemia. In some embodiments, administration of an anti-TMPRSS6 antibody result in increases hepcidin level in Diamond-Blackfan anemia subjects receiving blood transfusion. In some embodiments, administration of an anti-TMPRSS6 antibody result in reduced iron overload in Diamond-Blackfan anemia subjects receiving blood transfusion.

Determination of whether an amount of the antibody (e.g., anti-TMPRSS6 antibody) achieved the therapeutic effect would be evident to one of skill in the art based on the teachings provided herein. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. The particular dosage regimen, i.e., dose, timing and repetition, used in the method described herein will depend on the particular subject and that subject's medical history, as discussed herein.

Empirical considerations, such as time to maximum effect, the half-life, and/or time above a specific concentration generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Other reasons for dose-adjusting include differences in pharmacokinetics or pharmacodynamic response driven by sex, age, individual response, polymorphisms on the antibody target and/or receptors involved in antibody clearance. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

Dosing frequencies may vary in accordance with the claimed methods. In some embodiments, a composition may be administered once. In some embodiments, a composition will be administered on multiple occasions. In some embodiments, dosing frequency is every week, every 2 weeks, every 3 weeks, every 4 weeks, every 5 weeks, every 6 weeks, every 7 weeks, every 8 weeks, every 9 weeks, or every 10 weeks; or once every month, every 2 months, or every 3 months, or longer. In some embodiments, a composition will be administered daily, biweekly, weekly, bimonthly, monthly, or at any time interval that provide suitable (e.g., maximal) efficacy while minimizing safety risks to the subject. Generally, the efficacy and the treatment and safety risks may be monitored throughout the course of treatment.

In some embodiments, a subject may be administered a composition provided herein (e.g., an anti-TMPRSS6 antibody) at one or more intervals during a set period of time. In some cases, periods of time during which a subject is administered a composition at one or more intervals may be separated by periods of time in which the subject is not administered the composition. In some embodiments, the relative durations of respective periods of time may depend on the subject's response to treatment or severity of disease or both and/or may be determined based on the judgment of a treating physician.

In some embodiments, an antibody can be administered parenterally. For example, a parenterally administered composition may be administered topically, transmucosally, by subcutaneous, intracutaneous, intravenous, intraperitoneal, intramuscular, intraarticular, intraarterial, or infusion techniques.

In some embodiments, an antibody (e.g., an anti-TMPRSS6 antibody) is administered intravenously. In some embodiments, an antibody (e.g., an anti-TMPRSS6 antibody) is administered subcutaneously.

For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline. Ringer's solution or other suitable excipients. Other injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). In some cases, preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable, transdermal, or transmucosal depot sources of the antibody or local delivery systems.

An anti-TMPRSS6 antibody and treatment methods involving such as described in the present disclosure may be utilized in combination with other types of therapy for the target disease or disorder disclosed herein. In this context, an antibody composition and a therapeutic agent may be given either simultaneously or sequentially. Such therapies can be administered simultaneously or sequentially (in any order) with the treatment according to the present disclosure.

Accordingly, aspects of the disclosure related to methods and compositions (e.g., anti-TMPRSS6 antibodies). In some embodiments, antibodies as described herein, can be administered as a combination therapy (concomitantly or sequentially, e.g., over time). In some embodiments, the combination therapy comprises administering one or more of the antibodies as described herein (e.g., anti-TMPRSS6 antibodies) and at least one additional therapeutic agent (e.g., one, two, three, four, five, six, or seven therapeutic agents). In some embodiments, the antibodies are monospecific. In some embodiments, the antibodies are multi-specific. In some embodiments, one or more of the antibodies as described herein and at least one additional therapeutic agent (e.g., one, two, three, four, five, six, or seven therapeutic agents) are administered together. In some embodiments, one or more of the antibodies as described herein and at least one additional therapeutic agent (e.g., one, two, three, four, five, six, or seven therapeutic agents) are administered separately.

Any of the anti-TMPRSS6 antibodies disclosed herein can also be used for detecting presence of TMPRSS6 in vitro or in vivo. Results obtained from such detection methods can be used for diagnostic purposes (e.g., diagnosing diseases associated with TMPRSS6) or for scientific research purposes (e.g., identifying new TMPRSS6-secreting cell types, studying bioactivity and/or regulation of expressed TMPRSS6). For assay uses such as diagnostic uses, an anti-TMPRSS6 antibody as described herein may be conjugated with a detectable label (e.g., an imaging agent such as a contrast agent) for detecting presence of TMPRSS6, either in vivo or in vitro.

Any of the anti-TMPRSS6 antibodies disclosed herein can also be used for detecting presence of TMPRSS6 in vitro or in vivo. Results obtained from such detection methods can be used for diagnostic purposes (e.g., diagnosing diseases associated with TMPRSS6) or for scientific research purposes (e.g., studying bioactivity and/or regulation of secreted TMPRSS6). For assay uses such as diagnostic uses, an anti-TMPRSS6 antibody as described herein may be conjugated with a detectable label (e.g., an imaging agent such as a contrast agent) for detecting presence of TMPRSS6, either in vivo or in vitro.

As used herein, “conjugated” or “attached” means two entities are associated, preferably with sufficient affinity that the therapeutic/diagnostic benefit of the association between the two entities is realized. The association between the two entities can be either direct or via a linker, such as a polymer linker. Conjugated or attached can include covalent or noncovalent bonding as well as other forms of association, such as entrapment, e.g., of one entity on or within the other, or of either or both entities on or within a third entity, such as a micelle.

In other embodiments, an anti-TMPRSS6 antibody as described herein can be attached to a detectable label, which is a compound that is capable of releasing a detectable signal, either directly or indirectly, such that the aptamer can be detected, measured, and/or qualified, in vitro or in vivo. Examples of such “detectable labels” are intended to include, but are not limited to, fluorescent labels, chemiluminescent labels, colorimetric labels, enzymatic markers, radioactive isotopes, and affinity tags such as biotin. Such labels can be conjugated to the aptamer, directly or indirectly, by conventional methods.

The reporting agent can also be a dye, e.g., a fluorophore, which is useful in detecting a disease mediated by TMPRSS6 expressing cells in tissue samples respectively.

To perform a diagnostic assay in vitro, an anti-TMPRSS6 antibody can be brought in contact with a sample suspected of containing TMPRSS6, e.g., TMPRSS6 expressing cells in disease microenvironment. The antibody and the sample may be incubated under suitable conditions for a suitable period to allow for binding of the antibody to the TMPRSS6 antigen. Such an interaction can then be detected via routine methods, e.g., ELISA, histological staining or FACS. To perform a diagnostic assay in vivo, a suitable amount of anti-TMPRSS6 antibodies, conjugated with a label (e.g., an imaging agent or a contrast agent), can be administered to a subject in need of the examination. Presence of the labeled antibody can be detected based on the signal released from the label by routine methods.

To perform scientific research assays, an anti-TMPRSS6 antibody can be used to study bioactivity of TMPRSS6, detect the presence of TMPRSS6 on the cell surface, and or regulating the effect of TMPRSS6. For example, a suitable amount of anti-TMPRSS6 antibody can be brought in contact with a sample (e.g. a new cell type that is not previously identified as TMPRSS6 producing cells) suspected of expressing TMPRSS6. The antibody and the sample may be incubated under suitable conditions for a suitable period to allow for binding of the antibody to the TMPRSS6 antigen. Such an interaction can then be detected via routine methods, e.g., ELISA, histological staining or FACS.

### VI. Kits for Therapeutic and Diagnostic Applications

The present disclosure also provides kits for the therapeutic or diagnostic applications as disclosed herein. Such kits can include one or more containers comprising an anti-TMPRSS6 antibody, e.g., any of those described herein.

In some embodiments, the kit can comprise instructions for use in accordance with any of the methods described herein. The included instructions can comprise a description of administration of the anti-TMPRSS6 antibody to treat a target disease as those described herein. The kit may further comprise a description of selecting an individual suitable for treatment based on identifying whether that individual has the target disease. In still other embodiments, the instructions comprise a description of administering an antibody to an individual at risk of the target disease.

The instructions relating to the use of an anti-TMPRSS6 antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a disease or disorder. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like.

Also contemplated are packages for use in combination with a specific device, such as an infusion device, such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-TMPRSS6 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

Also provided herein are kits for use in detecting TMPRSS6 in a sample. Such a kit may comprise any of the anti-TMPRSS6 antibodies described herein. In some instances, the anti-TMPRSS6 antibody can be conjugated with a detectable label as those described herein. As used herein, “conjugated” or “attached” means two entities are associated, preferably with sufficient affinity that the therapeutic/diagnostic benefit of the association between the two entities is realized. The association between the two entities can be either direct or via a linker, such as a polymer linker. Conjugated or attached can include covalent or noncovalent bonding as well as other forms of association, such as entrapment, e.g., of one entity on or within the other, or of either or both entities on or within a third entity, such as a micelle.

Alternatively, or in addition, the kit may comprise a secondary antibody capable of binding to anti-TMPRSS6 antibody. The kit may further comprise instructions for using the anti-TMPRSS6 antibody for detecting TMPRSS6.

In order that the invention described herein may be more fully understood, the following example is set forth. The example described in this application is offered to illustrate the methods, devices, and systems provided herein and is not to be construed in any way as limiting their scope.

### EXAMPLES

### Example 1: Anti-TMPRSS6 Antibodies in the Treatment of a Mouse Model of Sickle Cell Disease

The Townes model of sickle cell disease (SCD) is used to test the efficacy of using the anti-TMPRSS6 antibodies of the present disclosure for treatment of SCD and its associated symptoms (e.g., erythrocyte sickling and hemolysis, vasoocclusive crisis (VOC) episodes and systemic and intravascular inflammation). Mice homozygous for the sickle BS form are referred to as HbSS mice; they are characterized by hemolytic anemia, high reticulocyte counts, and splenomegaly, and mimic many pathophysiological features of severe sickle cell disease (SCD) in humans. Mice homozygous for human BA are referred to as HbAA mice, are phenotypically normal, and are used as controls. Starting at 6~7-week-old, HbSS mice (n=10/group) are treated with either vehicle or anti-TMPRSS6 antibody via intraperitoneal (IP) dosing once per week for 8 weeks. Age-matched male and female HbAA mice are dosed with vehicle via IP once per week for 8 weeks, serving as controls.

Blood is collected from the mice at days 0, 14, 28, 42 and 56 for evaluation of hematological parameters including hemoglobin concentration and leukocytes. Animals are euthanized on day 56. Hemolysis markers (including lactate dehydrogenase, indirect bilirubin, total heme and hemopexin) are evaluated in plasma samples collected at day 56. Plasma concentrations on circulating adhesion molecules, including soluble platelet selectin (sP-selectin), soluble VCAM-1 (sVCAM-1), are determined in plasma samples collected at day 56.

The deformability of RBCs as a function of oxygen pressure is measured by oxygen gradient ektacytometry (oxygenscan) at day 56. Blood obtained by tail vein puncture is stored at 4° C. up to 24 hours before analysis. Deformability of red blood cells expressed as the elongation index (EI) is measured as a function of oxygen pressure using an oxygenscan test on a laser optical red cell rotational analyzer. Oxygenscan measurements are performed with 400×10⁶ red blood cells.

In order to conduct intravital microscopy analysis of vasoocclusion, dorsal skin-fold window chamber surgery is performed on anesthetized mice on day 28 (after 4 weeks of treatment with anti-TMPRSS6 antibody). Hemodynamics and blood cell adhesion to microvasculature are investigated by using intravital microscopy 90 minutes after induction of vasoocclusion by murine recombinant tumor necrosis factor-α (500 ng/mouse). Videos are recorded at different locations within the dorsal skin-fold window chamber to determine the effect of anti-TMPRSS6 antibody treatment on blood cell adhesion to the vascular endothelium and vasoocclusion. Cell adhesion is quantified by measuring the fluorescence intensity of adherent fluorescence-labeled cells on still images. Blood flow is determined by counting the number of vessels with normal blood flow, slow blood flow, and no blood flow according to frame-by-frame analysis of videos. Values obtained from analyzing all recorded vessel segments is averaged across all groups.

### Example 2: Single Dose of an Anti-TMPRSS6 Antibody Results in Dose-Dependent Effects in Normal Mice

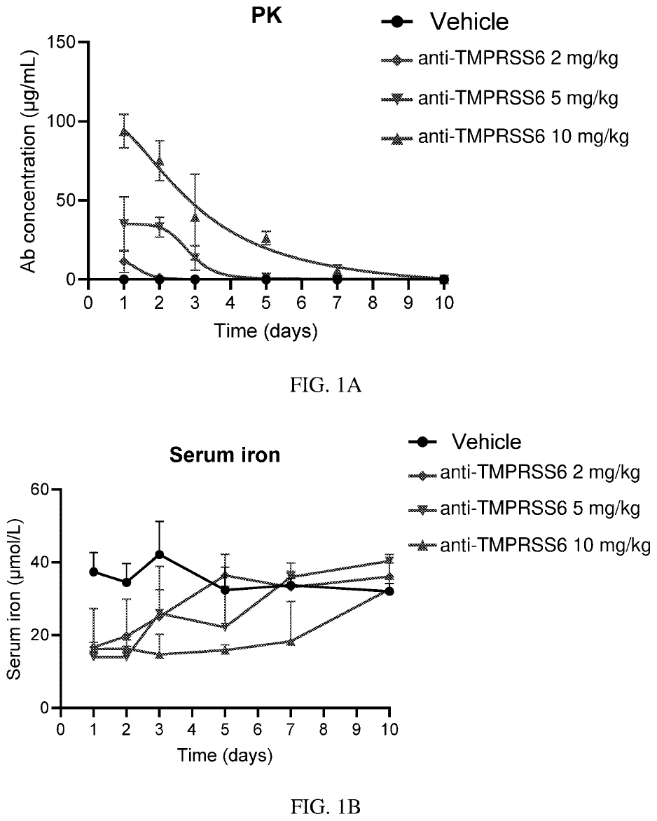

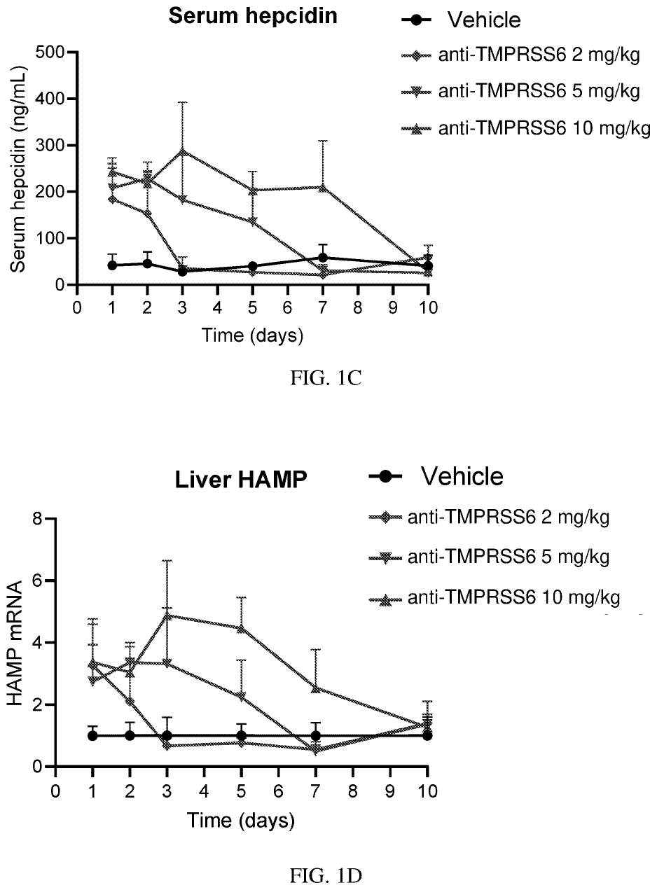

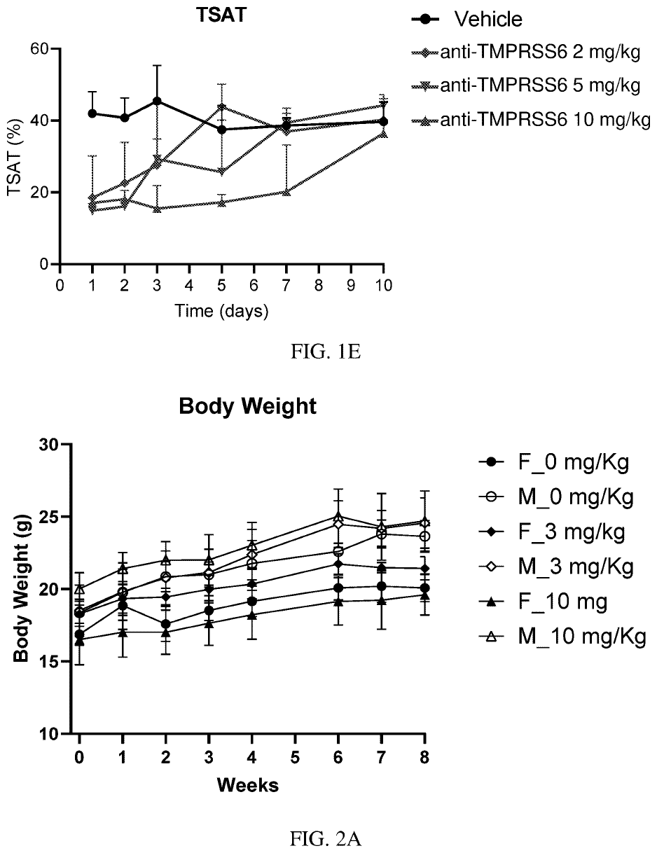

A study was carried out to evaluate the effects of dose-dependent TMPRSS6 inhibition on iron availability in mice. A single dose of an anti-TMPRSS6 antibody was administered to normal mice via intraperitoneal (IP) injection at doses of 0, 2, 5, or 10 mg/kg. A murine analog (i.e., surrogate) of the C3 antibody in Table 1 was developed for proof-of-concept studies in rodent models. The murine surrogate anti-TMPRSS6 antibody (referred to as anti-TMPRSS6 in the figures) comprises similar CDR sequences as the C3 antibody but has murinized framework and constant regions. Both antibodies have similar binding affinities to TMPRSS6. Cohorts of animals (n=5 per group) were euthanized on days 1, 2, 3, 5, 7, and 10 post-antibody administration. Levels of anti-TMPRSS6 antibody (FIG. 1A), iron (FIG. 1B), and hepcidin (FIG. 1C) were assessed in the serum from the mice. Inhibition of TMPRSS6 activity with the anti-TMPRSS6 antibody demonstrated dose-dependent upregulation of Hamp, the gene encoding hepcidin, in the liver (FIG. 1D). Furthermore, treatment with anti-TMPRSS6 increased serum hepcidin, which led to reduced serum iron and TSAT levels (FIG. 1E).

### Example 3. Anti-TMPRSS6 Antibody in the Treatment of Sickle Cell Disease

The Townes model of sickle cell disease (SCD) (see, e.g., Alvarez-Argote et al., Pathophysiological characterization of the Townes mouse model for sickle cell disease. Transl Res. 2023 April; 254:77-91) was used to test the efficacy of the anti-TMPRSS6 antibody for the treatment of SCD and its associated symptoms (e.g., hemolysis, systemic inflammation, complete blood count, and spleen and liver index). The Townes SCD mice carrying the homozygous sickle BS form are referred to as HbSS mice and are characterized by hemolytic anemia, high reticulocyte counts, and splenomegaly, and mimic many pathophysiological features of severe SCD in humans. Beginning at ~8 weeks old HbSS mice (n=10/group) were treated with either vehicle control or murinized anti-TMPRSS6 antibody at a dose of 3 or 10 mg/kg administered via IP injection once per week for 8 weeks.

Body weights of the animals were measured weekly throughout the course of the study. Peripheral blood was taken for complete blood count analysis on an Advia 2100 analyzer at the termination of the study (day 56). Hemolysis markers (including lactate dehydrogenase, direct bilirubin, and total bilirubin) were evaluated in plasma samples collected on day 56. Following euthanasia, spleen and liver weights were also measured.

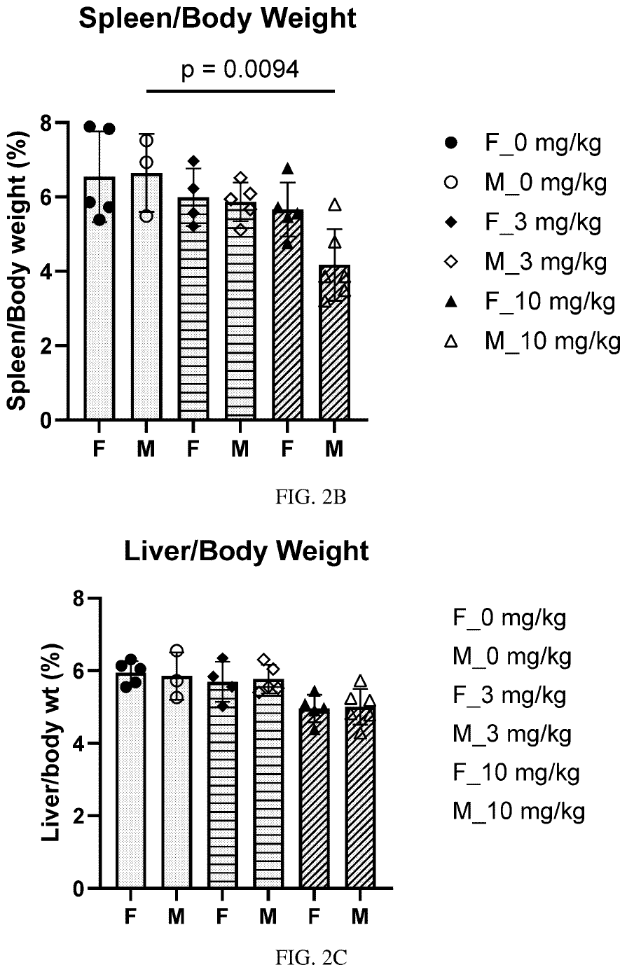

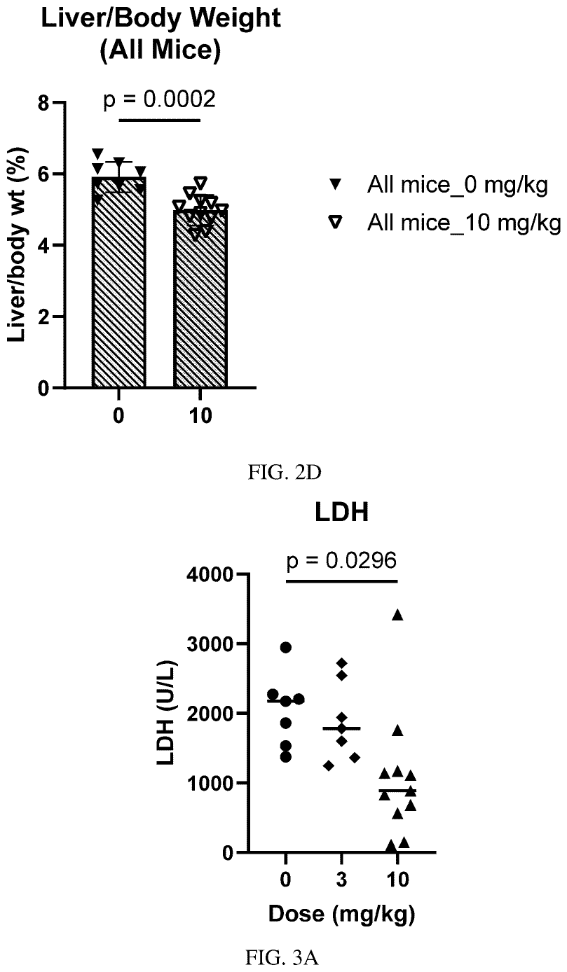

The HbSS mice gained body weight consistently for the duration of the studies (FIG. 2A). At the end of the study (day 56), spleen and liver weights were assessed and spleen and liver indices calculated (FIGS. 2B-2D, respectively). The spleen and liver indices of mice treated with anti-TMPRSS6 antibody trended lower, especially in the 10 mg/kg treatment group, indicating a reduction in extramedullary hematopoiesis and hepatomegaly in the mice treated with anti-TMPRSS6 antibody compared to vehicle control.

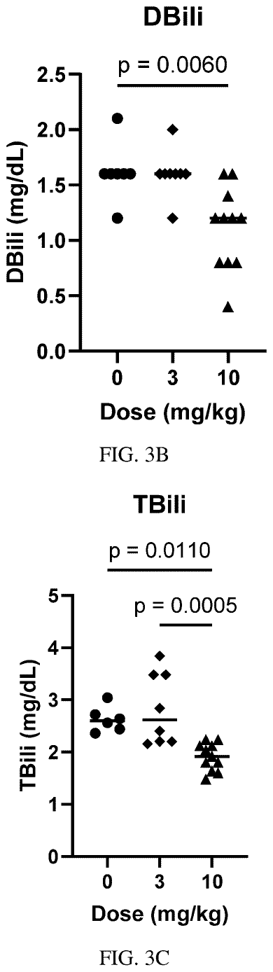

To evaluate the effect of anti-TMPRSS6 antibody on hemolysis, lactate dehydrogenase (LDH), direct bilirubin (DBili), and total bilirubin (TBili) levels were measured in the serum collected from the mice on day 56 of the study. Mice treated with 10 mg/kg anti-TMPRSS6 showed significant reduction in these markers of hemolysis, indicating a reduction of red blood cell lysis (FIGS. 3A-3C).

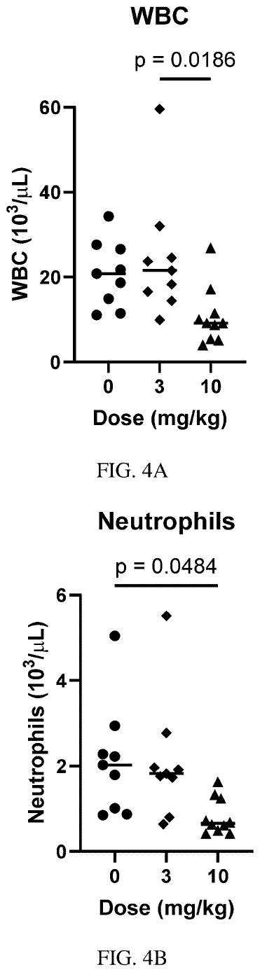

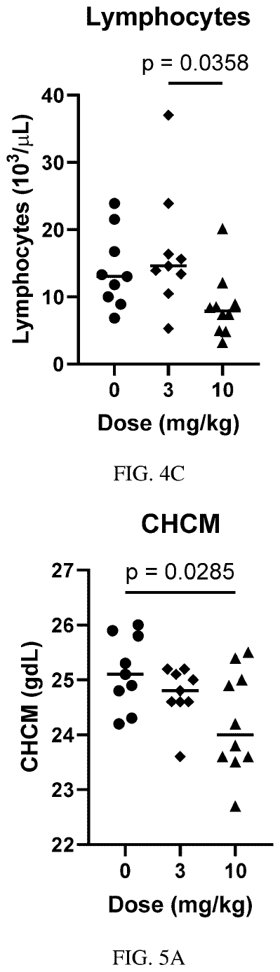

Complete blood count analysis was conducted using the peripheral blood from HbSS mice treated with anti-TMPRSS6 antibody or vehicle control. Mice treated with 10 mg/kg anti-TMPRSS6 showed significant reduction in white blood cells, including white blood cell count (WBC) (FIG. 4A), neutrophils (FIG. 4B), and lymphocytes (FIG. 4C). These results indicate that anti-TMPRSS6 reduced systemic inflammation in the mice with SCD. The WBC count was determined using a peroxidase assay.

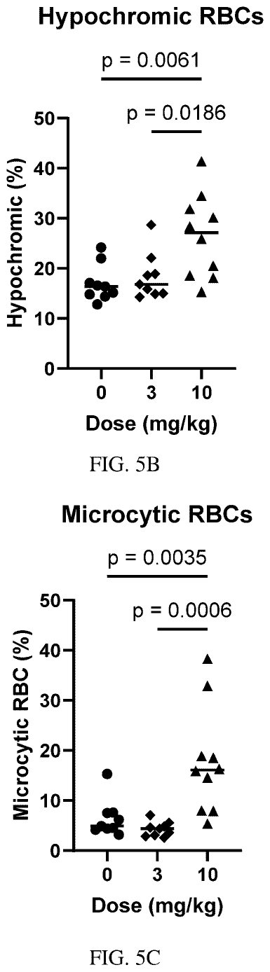

In addition to reduced inflammatory cells, treatment with anti-TMPRSS6 at 10 mg/kg reduced red blood cell HbS concentration (CHCM) in HbSS mice (FIG. 5A) as measured by an ADVIA hematology analyzer Shown in FIG. 5A is the mean of the optically measured hemoglobin concentration within red blood cells from each mouse group (i.e., per unit volume [g/dL]). This reduction was further evidenced by an increased percentage of circulating hypochromic red blood cells (RBCs) (FIG. 5B) and microcytic RBCs (FIG. 5C). This result is consistent with the proposed mechanism of action on treating SCD with an inhibitory anti-TMPRSS6 antibody: blocking the activity of TMPRSS6 with an anti-TMPRSS6 antibody leads to reduced HbS concentration, thus reducing HbS polymerization, and alleviates the disease symptoms associated with SCD.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also set forth as follows:
1. A method for treating sickle cell disease (SCD), the method comprising administering to a subject an effective amount of an anti-Transmembrane serine protease 6 (TMPRSS6) antibody.
2. The method of embodiment 1, wherein the antibody comprises a HC CDR1, HC CDR2 and HC CDR3 of a heavy chain variable domain having the amino acid sequence of SEQ ID NO: 49, and a LC CDR1, LC CDR2 and LC CDR3 of a light chain variable domain having the amino acid sequence of SEQ ID NO: 50.
3. The method of embodiment 1, wherein the antibody comprises a HC CDR1, HC CDR2 and HC CDR3 of a heavy chain variable domain having the amino acid sequence of SEQ ID NO: 7, and a LC CDR1, LC CDR2 and LC CDR3 of a light chain variable domain having the amino acid sequence of SEQ ID NO: 8.
4. The method of embodiment 1, wherein the antibody comprises a HC CDR1, HC CDR2 and HC CDR3 of a heavy chain variable domain having the amino acid sequence of SEQ ID NO: 19 or 78, and a LC CDR1, LC CDR2 and LC CDR3 of a light chain variable domain having the amino acid sequence of SEQ ID NO: 20.
5. The method of embodiment 1, wherein the antibody comprises a HC CDR1, HC CDR2 and HC CDR3 of a heavy chain variable domain having the amino acid sequence of SEQ ID NO: 30, and a LC CDR1, LC CDR2 and LC CDR3 of a light chain variable domain having the amino acid sequence of SEQ ID NO: 31.
6. The method of embodiment 1, wherein the antibody comprises a HC CDR1, HC CDR2 and HC CDR3 of a heavy chain variable domain having the amino acid sequence of SEQ ID NO: 37, and a LC CDR1, LC CDR2 and LC CDR3 of a light chain variable domain having the amino acid sequence of SEQ ID NO: 38.
7. The method of embodiment 1, wherein the antibody comprises a HC CDR1, HC CDR2 and HC CDR3 of a heavy chain variable domain having the amino acid sequence of SEQ ID NO: 44, and a LC CDR1, LC CDR2 and LC CDR3 of a light chain variable domain having the amino acid sequence of SEQ ID NO: 45.
8. The method of any one of embodiments 1-7, wherein the antibody comprises a HC CDR1 having the amino acid sequence of SEQ ID NO: 24, a HC CDR2 having the amino acid sequence of SEQ ID NO: 48, a HC CDR3 having the amino acid sequence of SEQ ID NO: 26, a LC CDR1 having the amino acid sequence of SEQ ID NO: 27, a LC CDR2 having the amino acid sequence of WAT, and a LC CDR3 having the amino acid sequence of SEQ ID NO: 29.
9. The method of any one of embodiments 1-7, wherein the antibody comprise a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 4, a LC CDR2 having the amino acid sequence of RAN, and a LC CDR3 having the amino acid sequence of SEQ ID NO: 6.
10. The method of any one of embodiments 1-7, wherein the antibody comprises a HC CDR1 having the amino acid sequence of SEQ ID NO: 13, a HC CDR2 having the amino acid sequence of SEQ ID NO: 14, a HC CDR3 having the amino acid sequence of SEQ ID NO: 15, a LC CDR1 having the amino acid sequence of SEQ ID NO: 16, a LC CDR2 having the amino acid sequence of WAF, and a LC CDR3 having the amino acid sequence of SEQ ID NO: 18.
11. The method of any one of embodiments 1-7, wherein the antibody comprises a HC CDR1 having the amino acid sequence of SEQ ID NO: 24, a HC CDR2 having the amino acid sequence of SEQ ID NO: 25, a HC CDR3 having the amino acid sequence of SEQ ID NO: 26, a LC CDR1 having the amino acid sequence of SEQ ID NO: 27, a LC CDR2 having the amino acid sequence of WAT, and a LC CDR3 having the amino acid sequence of SEQ ID NO: 29.
12. The method of any one of embodiments 1-7, wherein the antibody comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 35, a LC CDR1 having the amino acid sequence of SEQ ID NO: 36, a LC CDR2 having the amino acid sequence of RAN, and a LC CDR3 having the amino acid sequence of SEQ ID NO: 6.
13. The method of any one of embodiments 1-7, wherein the antibody comprises a HC CDR1 having the amino acid sequence of SEQ ID NO: 13, a HC CDR2 having the amino acid sequence of SEQ ID NO: 43, a HC CDR3 having the amino acid sequence of SEQ ID NO: 15, a LC CDR1 having the amino acid sequence of SEQ ID NO: 16, a LC CDR2 having the amino acid sequence of WAF, and a LC CDR3 having the amino acid sequence of SEQ ID NO: 18.
14. The method of any one of embodiments 1-13, wherein the antibody comprises a heavy chain variable domain having the amino acid sequence of SEQ ID NO: 49, and a light chain variable domain having the amino acid sequence of SEQ ID NO: 50.
15. The method of any one of embodiments 1-13, wherein the antibody comprises a heavy chain variable domain having the amino acid sequence of SEQ ID NO: 7, and a light chain variable domain having the amino acid sequence of SEQ ID NO: 8.
16. The method of any one of embodiments 1-13, wherein the antibody comprises a heavy chain variable domain having the amino acid sequence of SEQ ID NO: 19 or 78, and a light chain variable domain having the amino acid sequence of SEQ ID NO: 20.
17. The method of any one of embodiments 1-13, wherein the antibody comprises a heavy chain variable domain having the amino acid sequence of SEQ ID NO: 30, and a light chain variable domain having the amino acid sequence of SEQ ID NO: 31.
18. The method of any one of embodiments 1-13, wherein the antibody comprises a heavy chain variable domain having the amino acid sequence of SEQ ID NO: 37, and a light chain variable domain having the amino acid sequence of SEQ ID NO: 38.
19. The method of any one of embodiments 1-13, wherein the antibody comprises a heavy chain variable domain having the amino acid sequence of SEQ ID NO: 44, and a light chain variable domain having the amino acid sequence of SEQ ID NO: 45.
20. The method of any one of embodiments 1-19, wherein the antibody comprises a heavy chain comprising the amino acid sequence of SEQ ID NO: 52, and a light chain comprising the amino acid sequence of SEQ ID NO: 53.
21 The method of any one of embodiments 1-19, wherein the antibody comprises heavy chain comprising the amino acid sequence of SEQ ID NO: 11, and a light chain comprising the amino acid sequence of SEQ ID NO: 12.
22 The method of any one of embodiments 1-19, wherein the antibody comprises heavy chain comprising the amino acid sequence of SEQ ID NOs: 17 or 22, and a light chain comprising the amino acid sequence of SEQ ID NO: 23.
23. The method of any one of embodiments 1-19, wherein the antibody comprises heavy chain comprising the amino acid sequence of SEQ ID NO: 33, and a light chain comprising the amino acid sequence of SEQ ID NO: 34.
24. The method of any one of embodiments 1-19, wherein the antibody comprises heavy chain comprising the amino acid sequence of SEQ ID NO: 41, and a light chain comprising the amino acid sequence of SEQ ID NO: 42.
25. The method of any one of embodiments 1-19, wherein the antibody comprises heavy chain comprising the amino acid sequence of SEQ ID NO: 46, and a light chain comprising the amino acid sequence of SEQ ID NO: 47.
26. The method of any one of embodiments 1-25, wherein administration of the antibody reduces iron overload in the subject.
27. The method of any one of embodiments 1-26, wherein the administration of the antibody reduces hemolysis in the subject relative to the subject prior to the administration.
28. The method of any one of embodiments 1-27, wherein the subject has recurrent moderate to severe vasoocclusive crisis (VOC).
29. The method of any one of embodiments 1-28, wherein the administration of the antibody reduces frequency of VOC in the subject relative to the subject prior to the administration.
30. The method of any one of embodiments 1-19, wherein the administration reduces severity of VOC relative to the subject prior to the administration.
31. The method of embodiment 30, wherein the severity of VOC is measured by frequency of hospitalization and/or duration of hospitalization.
32. The method of any one of embodiments 1-31, wherein the administration of the antibody reduces systemic iron in the subject prior to the administration.
33. The method of any one of embodiments 1-32, wherein the administration of the antibody reduces mean corpuscular hemoglobin concentration (MCHC) relative to prior to the administration.
34. The method of any one of embodiments 1-33, wherein the administration reduces hemoglobin S (HbS) polymerization relative to the subject prior to the administration.
35. The method of any one of embodiments 1-34, wherein the administration reduces the frequency the subject needs blood transfusion relative to the subject prior to the administration.
36. The method of any one of embodiments 1-35, wherein the sickle cell disease is hemoglobin SS disease.
37. The method of any one of embodiments 1-36, wherein the sickle cell disease is hemoglobin SC disease.
38. The method of any one of embodiments 1-37, wherein the anti-TMPRSS6 antibody is administered as a co-therapy in combination hemoglobin S polymerization inhibitor (e.g., voxelotor), therapeutic agents for reducing VOC (e.g., Hydroxyurea, L-glutamine oral powder, Crizanlizumab, selective pyruvate kinase-R (PKR) activator), pain-relieving medications (e.g., narcotics, opioids, gabapentin, cannabis), a blood transfusion, a stem cell transplant, Exagmglogene autotemcel (exa-cel), LentiGlobin, GMI-1070, VIT-2763 (vamifeport), Ticagrelor, Vitamin D, simvastatin, AG-348 (mitapivat sulfate), propranolol, epinephrine, regadenoson, atorvastatin, prasugrel, L-arginine, OTQ923, oxygen therapy, or gene therapy.
39. A method for treating an iron overload associated condition in a subject in need thereof, wherein the subject does not have β-thalassemia intermedia or type I hemochromatosis, the method comprising administering to the subject an effective amount of an anti-Transmembrane serine protease 6 (TMPRSS6) antibody comprising: (a) a HC CDR1, HC CDR2 and HC CDR3 of a heavy chain variable domain having the amino acid sequence of SEQ ID NO: 49, and a LC CDR1, LC CDR2 and LC CDR3 of a light chain variable domain having the amino acid sequence of SEQ ID NO: 50; (b) a HC CDR1, HC CDR2 and HC CDR3 of a heavy chain variable domain having the amino acid sequence of SEQ ID NO: 7, and a LC CDR1, LC CDR2 and LC CDR3 of a light chain variable domain having the amino acid sequence of SEQ ID NO: 8; (c) a HC CDR1, HC CDR2 and HC CDR3 of a heavy chain variable domain having the amino acid sequence of SEQ ID NOs: 19 or 78, and a LC CDR1, LC CDR2 and LC CDR3 of a light chain variable domain having the amino acid sequence of SEQ ID NO: 20; (d) a HC CDR1, HC CDR2 and HC CDR3 of a heavy chain variable domain having the amino acid sequence of SEQ ID NO: 30, and a LC CDR1, LC CDR2 and LC CDR3 of a light chain variable domain having the amino acid sequence of SEQ ID NO: 31; (e) a HC CDR1, HC CDR2 and HC CDR3 of a heavy chain variable domain having the amino acid sequence of SEQ ID NO: 37, and a LC CDR1, LC CDR2 and LC CDR3 of a light chain variable domain having the amino acid sequence of SEQ ID NO: 38; or (f) a HC CDR1, HC CDR2 and HC CDR3 of a heavy chain variable domain having the amino acid sequence of SEQ ID NO: 44, and a LC CDR1, LC CDR2 and LC CDR3 of a light chain variable domain having the amino acid sequence of SEQ ID NO: 45.
40. The method of embodiment 39, wherein the anti-TMPRSS6 antibody comprises a HC CDR1, HC CDR2 and HC CDR3 of a heavy chain variable domain having the amino acid sequence of SEQ ID NO: 49, and a LC CDR1, LC CDR2 and LC CDR3 of a light chain variable domain having the amino acid sequence of SEQ ID NO: 50.
41. The method of embodiment 39 or 40, wherein the anti-TMPRSS6 antibody comprises: (a) a HC CDR1 having the amino acid sequence of SEQ ID NO: 24, a HC CDR2 having the amino acid sequence of SEQ ID NO: 48, a HC CDR3 having the amino acid sequence of SEQ ID NO: 26, a LC CDR1 having the amino acid sequence of SEQ ID NO: 27, a LC CDR2 having the amino acid sequence of WAT, and a LC CDR3 having the amino acid sequence of SEQ ID NO: 29; (b) a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 4, a LC CDR2 having the amino acid sequence of RAN, and a LC CDR3 having the amino acid sequence of SEQ ID NO: 6; (c) a HC CDR1 having the amino acid sequence of SEQ ID NO: 13, a HC CDR2 having the amino acid sequence of SEQ ID NO: 14, a HC CDR3 having the amino acid sequence of SEQ ID NO: 15, a LC CDR1 having the amino acid sequence of SEQ ID NO: 16, a LC CDR2 having the amino acid sequence of WAF, and a LC CDR3 having the amino acid sequence of SEQ ID NO: 18; (d) a HC CDR1 having the amino acid sequence of SEQ ID NO: 24, a HC CDR2 having the amino acid sequence of SEQ ID NO: 25, a HC CDR3 having the amino acid sequence of SEQ ID NO: 26, a LC CDR1 having the amino acid sequence of SEQ ID NO: 27, a LC CDR2 having the amino acid sequence of WAT, and a LC CDR3 having the amino acid sequence of SEQ ID NO: 29; (e) a HC CDR1 having the amino acid sequence of SEQ ID NO: 1, a HC CDR2 having the amino acid sequence of SEQ ID NO: 2, a HC CDR3 having the amino acid sequence of SEQ ID NO: 35, a LC CDR1 having the amino acid sequence of SEQ ID NO: 36, a LC CDR2 having the amino acid sequence of RAN, and a LC CDR3 having the amino acid sequence of SEQ ID NO: 6; or (f) a HC CDR1 having the amino acid sequence of SEQ ID NO: 13, a HC CDR2 having the amino acid sequence of SEQ ID NO: 43, a HC CDR3 having the amino acid sequence of SEQ ID NO: 15, a LC CDR1 having the amino acid sequence of SEQ ID NO: 16, a LC CDR2 having the amino acid sequence of WAF, and a LC CDR3 having the amino acid sequence of SEQ ID NO: 18.
42. The method of any one of embodiments 39-41, wherein the anti-TMPRSS6 antibody comprises a HC CDR1 having the amino acid sequence of SEQ ID NO: 24, a HC CDR2 having the amino acid sequence of SEQ ID NO: 48, a HC CDR3 having the amino acid sequence of SEQ ID NO: 26, a LC CDR1 having the amino acid sequence of SEQ ID NO: 27, a LC CDR2 having the amino acid sequence of WAT, and a LC CDR3 having the amino acid sequence of SEQ ID NO: 29.
43. The method of any one of embodiments 39-42, wherein the anti-TMPRSS6 antibody comprises: (a) a heavy chain variable domain having the amino acid sequence of SEQ ID NO: 49, and a light chain variable domain having the amino acid sequence of SEQ ID NO: 50; (b) a heavy chain variable domain having the amino acid sequence of SEQ ID NO: 7, and a light chain variable domain having the amino acid sequence of SEQ ID NO: 8; (c) a heavy chain variable domain having the amino acid sequence of SEQ ID NOs: 19 or 78, and a light chain variable domain having the amino acid sequence of SEQ ID NO: 20; (d) a heavy chain variable domain having the amino acid sequence of SEQ ID NO: 30, and a light chain variable domain having the amino acid sequence of SEQ ID NO: 31; (e) a heavy chain variable domain having the amino acid sequence of SEQ ID NO: 37, and a light chain variable domain having the amino acid sequence of SEQ ID NO: 38; or (f) a heavy chain variable domain having the amino acid sequence of SEQ ID NO: 44, and a light chain variable domain having the amino acid sequence of SEQ ID NO: 45.
44. The method of any one of embodiments 39-43, wherein the anti-TMPRSS6 antibody comprises a heavy chain variable domain having the amino acid sequence of SEQ ID NO: 49, and a light chain variable domain having the amino acid sequence of SEQ ID NO: 50.
45. The method of any one of embodiments 39-44, wherein the anti-TMPRSS6 antibody comprises: (a) a heavy chain comprising the amino acid sequence of SEQ ID NO: 52, and a light chain comprising the amino acid sequence of SEQ ID NO: 53; (b) a heavy chain comprising the amino acid sequence of SEQ ID NO: 11, and a light chain comprising the amino acid sequence of SEQ ID NO: 12; (c) a heavy chain comprising the amino acid sequence of SEQ ID NOs: 17 or 22, and a light chain comprising the amino acid sequence of SEQ ID NO: 23; (d) a heavy chain comprising the amino acid sequence of SEQ ID NO: 33, and a light chain comprising the amino acid sequence of SEQ ID NO: 34; (e) a heavy chain comprising the amino acid sequence of SEQ ID NO: 41, and a light chain comprising the amino acid sequence of SEQ ID NO: 42; or (f) a heavy chain comprising the amino acid sequence of SEQ ID NO: 46, and a light chain comprising the amino acid sequence of SEQ ID NO: 47.
46. The method of any one of embodiments 39-45, wherein the anti-TMPRSS6 antibody comprises a heavy chain comprising the amino acid sequence of SEQ ID NO: 52, and a light chain comprising the amino acid sequence of SEQ ID NO: 53.
47. The method of any one of embodiments 39-46, wherein administration of the antibody reduces systemic iron in the subject relative to prior to the administration.
48. The method of any one of embodiments 39-47, wherein the administration of the antibody reduces mean corpuscular hemoglobin concentration (MCHC) relative to prior to the administration.
49. The method of any one of embodiments 39-48, wherein the administration of the antibody reduces iron overload in the subject relative to prior to the administration.
50. The method of any one of embodiments 39-49, wherein the subject has hereditary hemochromatosis.
51. The method of embodiment 50, wherein the hereditary hemochromatosis is type 3 hereditary hemochromatosis.
52. The method of any one of embodiments 39-51, wherein the subject has or is suspected of having thalassemia.
53. The method of embodiment 52, wherein the thalassemia is α-thalassemia.
54. The method of embodiment 53, wherein the α-thalassemia is α-thalassemia major, α-thalassemia intermedia, or α-thalassemia-minor.
55. The method of embodiment 53, wherein the thalassemia is β-thalassemia.
56. The method of claim 55, wherein the β-thalassemia is β-thalassemia major, or β-thalassemia-minor.
57. The method of any one of embodiments 39-49, wherein the subject has or is suspected of having transfusion-related iron overload.
58. The method of embodiment 57, wherein the subject is receiving blood transfusion due to blood loss.
59. The method of embodiment 58, wherein the subject is receiving repeated blood transfusion due to anemia.
60. The method of any one of embodiments 39-49, wherein the subject has hemolytic anemia.
61. The method of embodiment 60, wherein the subject has transfusion-dependent hemolytic anemia, pyruvate kinase deficiency hemolytic anemia, thalassemia-related hemolytic anemia, or sickle cell disease-related hemolytic anemia.
62. The method of any one of embodiments 39-49, wherein the subject has African Iron Overload.
63. The method of any one of embodiments 39-49, wherein the subject has Diamond-Blackfan anemia.
64. The method of any one of embodiments 39-49, wherein the subject has myelodysplastic syndrome (MDS).
65. The method of embodiment 64, wherein the MDS is refractory anemia with ring-sideroblasts (RARS).
66. The method of embodiment 64 or 65, wherein the subject has a SF3B1 mutation.

1-21. (canceled)

22. A kit for treating an iron overload-associated condition in a subject in need thereof, the kit comprising:
(a) a container comprising a pharmaceutical composition comprising an anti-transmembrane serine protease 6 (TMPRSS6) antibody or an antigen-binding fragment thereof,
wherein the anti-TMPRSS6 antibody comprises the HC CDR1, HC CDR2, HC CDR3, LC CDR1, LC CDR2, and LC CDR3 of any one of the antibodies set forth in Table 1; and
(b) instructions for administering the pharmaceutical composition to the subject.
